(19) United States
(12) Patent Application Publication
(10) Pub. No.: US 2025/0235434 A1
(43) Pub. Date: Jul. 24, 2025

(54) RIFABUTIN ANALOGS FOR THE TREATMENT OF DISEASE

(71) Applicant: BIOVERSYS AG, Basel (CH)

(72) Inventors: Kevin ANTRAYGUES, Loos (FR); Marilyne BOUROTTE, Perenchies (FR); Glenn E. DALE, Basel (CH); Olivier DEFERT, Lille (FR); Marc GITZINGER, Basel (CH); Sergio LOCIURO, Basel (CH); Mathieu MAINGOT, Lille (FR); Vincent TREBOSC, Kembs (FR); Nicolas WILLAND, Lille (FR)

(21) Appl. No.: 18/855,839

(22) PCT Filed: Apr. 6, 2023
(86) PCT No.: PCT/EP2023/059154

§ 371 Date: Oct. 10, 2024

(30) Foreign Application Priority Data
Apr. 11, 2022 (EP) 22167670.3

(51) Int. Cl. A61K 31/438; A61K 31/4545; A61K 31/496; A61K 31/5377; C07D 498/22; C07D 519/00

(52) U.S. Cl. CPC A61K 31/438; A61K 31/4545; A61K 31/496; A61K 31/5377; C07D 498/22; C07D 519/00

(57) ABSTRACT

The present invention relates to compounds and pharmaceutical compositions comprising the same for the treatment, amelioration and/or prevention of disease. In some embodiments, the disease is a bacterial infection. In some embodiments, the bacterial infection is caused by one or more bacterium belonging to a genus of non-tuberculous Mycobacteria, preferably M. abscessus.

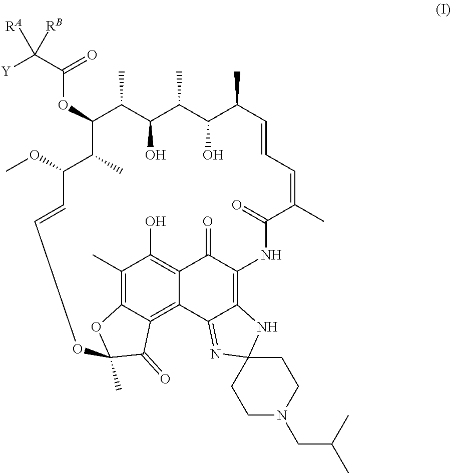

The present invention relates to compounds and pharmaceutical compositions comprising the same for the treatment, amelioration and/or prevention of disease. In some embodiments, the disease is a bacterial infection. In some embodiments, the bacterium belongs to the genus or species Clostridium spp., Enterococcus spp., Hemophilus spp., Legionella spp., Mycobacterium spp., Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii. In some embodiments the infection is caused by a A. baumannii, and/or S. aureus, and/or a genus of non-tuberculous Mycobacteria (NTM), preferably M. abscessus.

### RELATED ART

Rifamycins such as rifabutin are known antibiotics with activity against a broad spectrum of pathogens such as Clostridium spp., Enterococcus spp., Hemophilus spp., Legionella spp., Mycobacterium spp. (tuberculous and non-tuberculous Mycobacteria), Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and Toxoplasma gondii (Kunin, Clin. Infect. Dis., 1996, 22 (suppl 1):S3-14; Farr and Mandell, Med. Clin. North. Am., 1982; Thomsberry et al., Rev. Infect. Dis., 1983; Hoover et al., Diagn. Microbiol. Infect. Dis., 1993; Kerry et al., J. Antimicrob. Chemother., 1975).

Rifabutin has been recently shown to have potent in vitro and in vivo activity against Mycobacterium abscessus (Aziz et al., Antimicrob. Agents Chemother., 2017; Dick et al., Antimicrob. Agents Chemother., 2020) and Acinetobacter baumannii (Luna et al., Nat. Microbiol., 2020; Trebosc et al., Drug Discov. Today, 2021, 26(9): 2099-2014; Trebosc et al., J. Antimicrob. Chemother., 2020).

Derivatives of rifabutin have also shown activity against M. avium and M. tuberculosis (WO 2004/005298).

However, there remains a need for more effective rifamycins for the treatment of bacterial infections such as M. abscessus and A. baumannii infections.

### SUMMARY OF THE INVENTION

In one aspect, the present invention provides a compound of the Formula I or a pharmaceutically acceptable salt, tautomer, solvate, hydrate or enantiomer thereof:

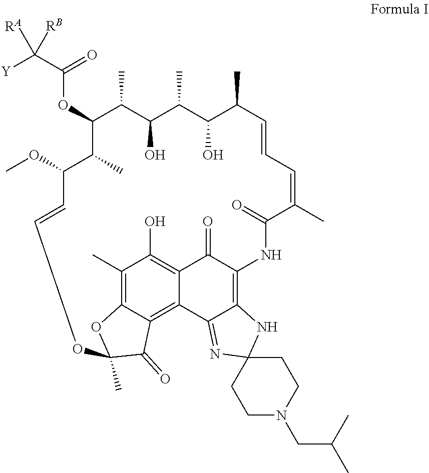

wherein:
R^{A} and R^{B} are each independently —H, —C₁-C₆ alkyl, or C₃-C₆ cycloalkyl;
Y is —NR¹R² or [NR⁵¹R⁵²R⁵³]⁺X⁻;
R¹ and R² are each independently —H, —C₁-C₆ alkyl, —R³, or —C₁-C₆-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₆ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-15 membered heterocycloalkyl, wherein said heterocycloalkyl is optionally substituted with one or more —C₁-C₆ alkyl, halogen, —OR¹⁰, —NR¹¹R¹², oxo, —R¹³, or —C₁-C₆ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each —C₁-C₆ alkyl and —C₆-C₁₀ aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently a pharmaceutically acceptable anion, preferably X⁻ is F⁻, Cl⁻, Br⁻, I⁻, F₃CSO₃⁻, or H₃CC₆H₄SO₃⁻.

In one aspect, the present invention provides a pharmaceutical composition comprising a compound of Formula I as described herein, or a pharmaceutically acceptable salt thereof, and a pharmaceutically acceptable carrier. In some preferred embodiments, the pharmaceutical composition is effective for treating a bacterial infection. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a compound of Formula I or a pharmaceutically acceptable salt thereof as described herein for use as a medicament. In another aspect, the present invention provides a compound of Formula I or a pharmaceutically acceptable salt thereof as described herein for use in the prevention or treatment of a bacterial infection. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a use of a compound of Formula I or pharmaceutical composition comprising a compound of Formula I as described herein in the manufacture of a medicament for treating a bacterial infection. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In one aspect, the present invention provides a method of treating a bacterial infection in a subject in need thereof, the method comprising administering to the subject a therapeutically effective amount of a compound of Formula I as described herein or a pharmaceutically acceptable salt thereof. In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

The present invention provides rifabutin analogs that are modified at the C25 position and pharmaceutical compositions thereof. The inventive compounds exhibit broad antibacterial activity against a wide array of bacterial species, and thus maintain the broad antibacterial activity characteristic of the rifamycin class of antibiotics. Additional features and advantages of the present technology will be apparent to one of skill in the art upon reading the Detailed Description, below.

### DETAILED DESCRIPTION

The present invention provides analogs of rifabutin that are effective in treating bacterial infections, preferably bacterial infections caused by one or more bacterium belonging to a genus of non-tuberculous Mycobacteria, preferably M. abscessus.

The details of the technology are set forth in the accompanying description below. Although methods and materials similar or equivalent to those described herein can be used in the practice or testing of the present technology, illustrative methods and materials are now described. Other features, objects, and advantages of the invention will be apparent from the description and from the claims.

### Definitions

Unless defined otherwise, all technical and scientific terms used herein have the same meanings as commonly understood by one of ordinary skill in the art to which this invention belongs.

The articles “a” and “an” are used in this disclosure to refer to one or more than one (i.e., to at least one) of the grammatical object of the article unless the context clearly dictates otherwise. By way of example, “an element” means one element or more than one element.

The term “and/or” is used in this disclosure to mean either “and” or “or” unless indicated otherwise.

The term “optionally substituted” is understood to mean that a given chemical moiety (e.g. an alkyl group) can (but is not required to) be bonded other substituents (e.g. heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups.

The term “alkyl” refers to a straight or branched chain saturated hydrocarbon. C₁-C₆ alkyl groups contain 1 to 6 carbon atoms. Examples of a —C₁-C₆ alkyl group include, but are not limited to, methyl, ethyl, propyl, butyl, pentyl, isopropyl, isobutyl, sec-butyl and tert-butyl, isopentyl and neopentyl.

The terms “alkylene” or “alkylenyl,” as used herein, refer to a straight or branched hydrocarbon chain bi-radical derived from alkyl, as defined herein, wherein one hydrogen of said alkyl is cleaved off generating the second radical of said alkylene. Examples of alkylene are, by way of illustration, —CH₂—, —CH₂—CH₂—, —CH(CH₃)—, —CH₂—CH₂—CH₂—, —CH(CH₃)—CH₂—, or —CH(CH₂CH₃)—.

The term “cycloalkyl” means monocyclic or polycyclic saturated carbon rings containing 3-6 carbon atoms. Examples of cycloalkyl groups include, without limitations, cyclopropyl, cyclobutyl, cyclopentyl, and cyclohexyl.

The term “aryl” refers to cyclic, aromatic hydrocarbon groups that have 1 to 2 aromatic rings, including monocyclic or bicyclic groups such as phenyl, biphenyl or naphthyl. A C₆-C₁₀ aryl group contains between 6 and 10 carbon atoms. When containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—C₁-C₆ alkyl, —C₁-C₆ alkyl, —OH, —NH₂, —NH(C₁-C₆ alkyl), and —N(C₁-C₆ alkyl)₂. The substituents (e.g., alkyl groups) can themselves be optionally substituted.

Unless otherwise specifically defined, “heteroaryl” means a monovalent monocyclic or bicyclic aromatic radical of 5 to 15 ring atoms containing one or more ring heteroatoms selected from N, S, P, and O, the remaining ring atoms being C. Preferably the heteroatom is selected from N, S, and O, more preferably N and O. A 5-10 membered heteroaryl group contains between 5 and 10 atoms. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, quinolyl, isothiazolyl, thiazolyl, thiadiazole, indazole, benzimidazolyl, 1,3-dihydro-2H-benzimidazol-2-one, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, benzofuran, quinolinyl, isoquinolinyl, 1,6-naphthyridinyl, thieno[2,3-b]pyrazinyl, quinazolinyl, tetrazolo[1,5-a]pyridinyl, [1,2,4]triazolo[4,3-a]pyridinyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, pyridin-2-one, furo[3,2-c]pyridinyl, furo[2,3-c]pyridinyl, benzooxazolyl, benzisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, thiazolo[5,4-d]thiazolyl, imidazo[2,1-b][1,3,4]thiadiazolyl, and thieno[2,3-b]pyrrolyl.

The terms “heterocyclyl” or “heterocycloalkyl” or “heterocycle” refer to monocyclic or polycyclic saturated or partially saturated 3 to 15-membered ring systems containing carbon and heteroatoms taken from O, N, and S (preferably O and N) and wherein at least one ring does not comprise delocalized π electrons (aromaticity) shared among the ring carbon or heteroatoms. A 3-10 membered heterocycloalkyl group contains between 3 and 10 atoms. Heterocyclyl rings include, but are not limited to, oxetanyl, azetadinyl, tetrahydrofuranyl, pyrrolidinyl, oxazolinyl, oxazolidinyl, thiazolinyl, thiazolidinyl, pyranyl, thiopyranyl, tetrahydropyranyl, piperidinyl, morpholinyl, thiomorpholinyl, thiomorpholinyl S-oxide, thiomorpholinyl S-dioxide, piperazinyl, azepinyl, oxepinyl, [1,4]diazepane, [1,2]diazepane, decahydro-[1,6]naphthyridine and diazepinyl.

A heterocyclyl or heterocycloalkyl ring can also be fused or bridged, e.g., can be a bicyclic or tricyclic ring. Furthermore, when containing two or more fused rings, the heterocycloalkyl groups herein defined can have an unsaturated or partially saturated ring fused with an aromatic and/or heteroaromatic ring. Exemplary ring systems of such heterocyle-aryl or heterocyle-heteroaryl groups, which are understood herein as embodiments of heterocyclyl groups, include indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuran, 2,3,4,5-tetrahydro-1H-pyrido[4,3-b]indole, 5,6,7,8-tetrahydro-imidazo[1,2-a]pyrazine, and dihydrobenzoxanyl.

A heterocyclyl or heterocycloalkyl ring can also be a spirocyclic heterocycle or spiroheterocycle. As used herein a spirocyclic heterocycle or spiroheterocycle is understood to mean a bicyclic or multicyclic ring system in which at least two rings are connected through a single atom, and wherein at least one of the rings is a heterocycle (e.g., at least one of the rings is furanyl, morpholinyl, or piperadinyl). One or both of the rings in a spiroheterocycle can be can be fused to one or more additional carbocyclic, heterocyclic, aromatic, or heteroaromatic ring to form, e.g., a tricyclic ring system in which two of the rings are connected through a single atom. An exemplary spirocyclic heterocycle of the present invention is 1,3,8-triaza-spiro[4.5]decane.

As used herein, the term “halo” or “halogen” means fluoro (F), chloro (Cl), bromo (Br), or iodo (I).

The term “oxo” refers to a carbonyl functional group composing a carbon atom double-bonded to an oxygen atom. It can be abbreviated herein as “oxo”, as C(O), or as C═O.

The invention also includes pharmaceutical compositions comprising an effective amount of a disclosed compound or a pharmaceutically acceptable salt thereof. Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, hydroiodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. In some embodiments, wherein Y is [NR⁵¹R⁵²R⁵³]⁺, X⁻ can be a pharmaceutically acceptable anion, e.g., X⁻ can be any of the anionic species listed above.

The term “stereoisomers” refers to the set of compounds which have the same number and type of atoms and share the same bond connectivity between those atoms, but differ in three-dimensional structure. The term “stereoisomer” refers to any member of this set of compounds.

The term “diastereomers” refers to the set of stereoisomers which cannot be made superimposable by rotation around single bonds. For example, cis- and trans-double bonds, endo- and exo-substitution on bicyclic ring systems, and compounds containing multiple stereogenic centers with different relative configurations are considered to be diastereomers. The term “diastereomer” refers to any member of this set of compounds. In some examples presented, the synthetic route may produce a single diastereomer or a mixture of diastereomers. In some cases these diastereomers were separated and in other cases a wavy bond is used to indicate the structural element where configuration is variable.

The term “enantiomers” refers to a pair of stereoisomers which are non-superimposable mirror images of one another. The term “enantiomer” refers to a single member of this pair of stereoisomers. The term “racemic” refers to a 1:1 mixture of a pair of enantiomers.

The term “tautomers” refers to a set of compounds that have the same number and type of atoms, but differ in bond connectivity and are in equilibrium with one another. A “tautomer” is a single member of this set of compounds. Typically a single tautomer is drawn but it is understood that this single structure is meant to represent all possible tautomers that might exist. Examples include enol-ketone tautomerism. When a ketone is drawn it is understood that both the enol and ketone forms are part of the present disclosure.

The term “solvate” refers to a complex of variable stoichiometry formed by a solute and solvent. Such solvents for the purpose of the invention may not interfere with the biological activity of the solute. Examples of suitable solvents include, but are not limited to, water, MeOH, EtOH, and AcOH. Solvates wherein water is the solvent molecule are typically referred to as “hydrates.” Hydrates include compositions containing stoichiometric amounts of water, as well as compositions containing variable amounts of water.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject, or administering a prodrug derivative or analog of the compound or pharmaceutically acceptable salt of the compound or composition to the subject, which can form an equivalent amount of active compound within the subject's body.

A “patient” or “subject” is a mammal, e.g., a human, mouse, rat, guinea pig, dog, cat, horse, cow, pig, or non-human primate, such as a monkey, chimpanzee, baboon or rhesus.

### Compounds of the Invention

In one aspect, the present invention provides a compound of the Formula I or a pharmaceutically acceptable salt, tautomer, solvate, hydrate or enantiomer thereof:

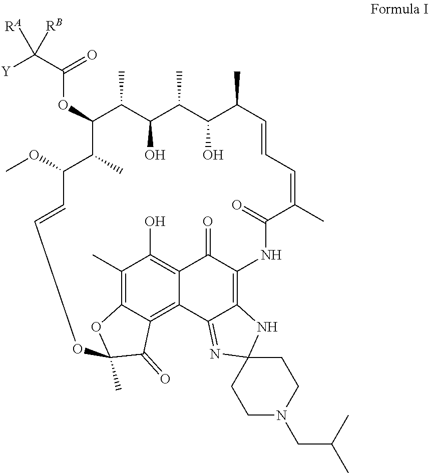

wherein:
R^{A} and R^{B} are each independently —H, —C₁-C₆ alkyl, or C₃-C₆ cycloalkyl;
Y is —NR¹R² or [NR⁵R⁵²R⁵³]⁺X⁻
R¹ and R² are each independently —H, —C₁-C₆ alkyl, —R³, or —C₁-C₆-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₆ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-15 membered heterocycloalkyl, wherein said heterocycloalkyl is optionally substituted with one or more —C₁-C₆ alkyl, halogen, —OR¹⁰, —NR¹¹R¹², oxo, —R¹³, or —C₁-C₆ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each —C₁-C₆ alkyl and —C₆-C₁₀ aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently a pharmaceutically acceptable anion, preferably X⁻ is F⁻, Cl⁻, Br⁻, I⁻ F₃CSO₃⁻, or H₃CC₆H₄SO₃⁻.

In some embodiments, R^{A} and R^{B} are each independently —H, —C₁-C₆ alkyl, or C₃-C₆ cycloalkyl;
Y is —NR¹R² or [NR⁵¹R⁵²R⁵³]⁺X⁻
R¹ and R² are each independently —H, —C₁-C₆ alkyl, —R³, or —C₁-C₆-alkylene-R^{3A}, wherein said alkyl and said alkylene are each independently optionally substituted with one or more halogen;
R³ is a 3-10 membered heterocycloalkyl, or C₆-C₁₀ aryl, wherein each heterocycloalkyl or aryl is optionally substituted with one or more halogen or —C₁-C₆ alkyl, wherein said alkyl is optionally substituted with one or more halogen;
R^{3A} is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₆ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-15 membered heterocycloalkyl, wherein said heterocycloalkyl is optionally substituted with one or more —C₁-C₆ alkyl, halogen, —OR¹⁰, —NR¹¹R¹², oxo, —R¹³, or —C₁-C₆ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each —C₁-C₆ alkyl and —C₆-C₁₀ aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently F⁻, Cl⁻, Br⁻, I⁻, F₃CSO₃⁻, or H₃CC₆H₄SO₃⁻.

In some embodiments, R^{A} and R^{B} are each independently —H, —C₁-C₆ alkyl, or C₃-C₆ cycloalkyl;
Y is —NR¹R² or [NR⁵¹R⁵²R⁵³]⁺X⁻
R¹ and R² are each independently —H, —C₁-C₆ alkyl, —R³, or —C₁-C₆-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₆ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-10 membered heterocycloalkyl, wherein said heterocycloalkyl is optionally substituted with one or more —C₁-C₆ alkyl, halogen, —OR¹⁰, —NR¹¹R¹², oxo, —R¹³, or —C₁-C₆ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each —C₁-C₆ alkyl and —C₆-C₁₀ aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently F⁻, Cl⁻, Br⁻, I⁻, F₃CSO₃⁻, or H₃CC₆H₄SO₃⁻.
In some embodiments, R^{A} and R^{B} are each independently H, —C₁-C₆ alkyl, or C₅-C₆ cycloalkyl. In some embodiments, R^{A} and R^{B} are each independently —H or —C₁-C₆ alkyl. In some embodiments, R^{A} and R^{B} are each independently —H or —C₁-C₄ alkyl. In some embodiments, R^{A} and R^{B} are each independently —H or —C₁-C₂ alkyl. In some preferred embodiments, R^{A} and R^{B} are each —H.

In some embodiments, R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently selected from —H and —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently F⁻, Cl⁻, Br⁻, I⁻, F₃CSO₃⁻, or H₃CC₆H₄SO₃⁻.

In some embodiments, R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently selected from —H and —C₁-C₄ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₄ alkyl, —OC₁-C₄ alkyl, or —N(C₁-C₄ alkyl)₂; and
X⁻ is independently F⁻, Cl⁻, Br⁻, I⁻, F₃CSO₃⁻, or H₃CC₆H₄SO₃⁻.

In some embodiments, R¹ and R² are each independently —H, —C₁-C₄ alkyl, —R³, or —C₁-C₄-alkylene-R³;
R³ is a 3-10 membered heterocycloalkyl, C₆-C₁₀ aryl, —OR⁴, or —NR⁵R⁶, wherein each heterocycloalkyl or aryl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently selected from —H and —C₁-C₄ alkyl;
or R⁵¹, R¹² and R⁵³, together with the nitrogen atom to which they are attached, combine to form a 5 to 10-membered ring heteroaryl, wherein said heteroaryl is optionally substituted with one or more halogen, —C₁-C₄ alkyl, —OC₁-C₄ alkyl, or —N(C₁-C₄ alkyl)₂; and
X⁻ is independently F⁻, Cl⁻, Br⁻, I⁻, F₃CSO₃⁻, or H₃CC₆H₄SO₃⁻.

In some embodiments, R¹ and R² are each independently —H, —R³, or —C₁-C₄-alkylene-R³;
R³ is a 3-10 membered heterocycloalkyl, C₆-C₁₀ aryl, —OR⁴, or —NR⁵R⁶, wherein each heterocycloalkyl or aryl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or —C₆-C₁₀ aryl, wherein each alkyl or aryl is optionally substituted with one or more halogen;
R¹¹ and R¹² are each independently —H, —C₁-C₄ alkyl, —CO₂—C₁-C₄ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl;
R⁵¹, R⁵² and R⁵³ are each independently selected from —H and —C₁-C₄ alkyl;
or R⁵¹, R⁵² and R⁵³, together with the nitrogen atom to which they are attached, combine to form a 1-pyridinium, wherein said 1-pyridinium is optionally substituted with one or more halogen, —C₁-C₄ alkyl, —OC₁-C₄ alkyl, or —N(C₁-C₄ alkyl)₂; and
X⁻ is independently F⁻, Cl⁻, Br⁻, I⁻, F₃CSO₃; or H₃CC₆H₄SO₃⁻.

In some embodiments, R¹ and R² are each independently —H, —R³, or —C₁-C₄-alkylene-R³;
R³ is a 5-6 membered heterocycloalkyl, phenyl, —OR⁴, or —NR⁵R⁶, wherein each heterocycloalkyl or phenyl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or phenyl, wherein each alkyl or phenyl is optionally substituted with one or more halogen;
R¹¹ and R¹² are each independently —H, —C₁-C₄ alkyl, —CO₂—C₁-C₄ alkyl, phenyl, or —SO₂-phenyl; wherein each alkyl or phenyl is optionally substituted with one or more halogen;
R¹³ is a 3-15 membered heterocycloalkyl, 5- to 10-membered heteroaryl, or C₆-C₁₀ aryl; wherein each heterocycloalkyl, heteroaryl, or aryl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl;
R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more halogen, —C₁-C₂ alkyl, —OC₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, R¹ and R² are each independently —H, —R³, or —C₁-C₄-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or phenyl;
R¹¹ and R¹² are each independently —H, —C₁-C₄ alkyl optionally substituted with one or more halogen, —CO₂-tert-butyl, phenyl optionally substituted with one or more halogen, or —SO₂-phenyl;
R¹³ is a 3-15 membered heterocycloalkyl, 5- to 10-membered heteroaryl, or phenyl; wherein each heterocycloalkyl, heteroaryl, or phenyl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl;
R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, R¹ and R² are each independently —H, —R³, or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ and R¹² are each independently —H, —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heterocycloalkyl, heteroaryl, or phenyl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl;
R⁵¹, R¹² and R⁵³ are each independently selected from —H and —C₁-C₂ alkyl;
or R⁵¹, R¹² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, R¹ is H;
R² is independently —H, —R³, or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is phenyl;
R¹¹ H or —C₁-C₂ alkyl;
R¹² is independently —H, —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heterocycloalkyl or heteroaryl is optionally substituted with one or more oxo, halogen, —OR¹⁴, or —C₁-C₄ alkyl, wherein each alkyl is optionally substituted with one or more halogen, and wherein each phenyl is optionally substituted with one or more halogen;
R¹⁴ is independently —H or —C₁-C₄ alkyl;
R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments,
R¹ is H;
R² is independently —R³ or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heteroaryl is optionally substituted with one or more oxo, halogen, or —C₁-C₄ alkyl, wherein said alkyl is optionally substituted with one or more halogen, and wherein said phenyl is optionally substituted with one or more halogen, —OR¹⁴ or —C₁-C₄ alkyl, wherein each alkyl is optionally substituted with one or more halogen;
R¹⁴ is independently —H or —C₁-C₄ alkyl;
R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, R¹ is H;
R² is independently —R³ or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl;
R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, R¹ is H;
R² is independently —R³ or —C₁-C₃-alkylene-R³;
R³ is a 1-piperidine, 4-piperidine, 1-piperazine, 4-morpholine, phenyl, —OR⁴, or —NR⁵R⁶, wherein each 1-piperidine is optionally substituted with one or more —C₁-C₄ alkyl at the 4-position, each 4-piperidine is optionally substituted with one or more —C₁-C₄ alkyl at the 1-position, and each 1-piperazine is optionally substituted with one or more —C₁-C₄ alkyl at the 4-position;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl;
R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, R¹ is H;
R² is independently —R³ or —C₂-C₃-alkylene-R³;
R³ is a 1-piperidine, 4-morpholine, or —NR⁵R⁶, wherein each piperidine is optionally substituted with one or more —C₁-C₂ alkyl at the 4-position;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl;
R⁵¹, R¹² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, R¹ is H;
R² is independently —R³ or —C₂-C₃-alkylene-R³;
R³ is a 1-piperidine, 4-morpholine, or —NR⁵R⁶;
R⁵ and R⁶ are independently, at each occurrence, —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl;
R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some preferred embodiments, Y is —NR¹R².

In some embodiments, Y is —NR¹R²;
R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl.

In some embodiments, Y is —NR¹R²; R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl.

In some embodiments, Y is —NR¹R²;
R¹ and R² are each independently —H, —C₁-C₄ alkyl, —R³, or —C₁-C₄-alkylene-R³;
R³ is a 3-10 membered heterocycloalkyl, C₆-C₁₀ aryl, —OR⁴, or —NR⁵R⁶, wherein each heterocycloalkyl or aryl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen; and
R¹⁴ is independently —H or —C₁-C₆ alkyl.

In some embodiments, Y is —NR¹R²;
R¹ and R² are each independently —H, —R³, or —C₁-C₄-alkylene-R³;
R³ is a 3-10 membered heterocycloalkyl, C₆-C₁₀ aryl, —OR⁴, or —NR⁵R⁶, wherein each heterocycloalkyl or aryl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or —C₆-C₁₀ aryl, wherein each alkyl or aryl is optionally substituted with one or more halogen;
R¹¹ and R¹² are each independently —H, —C₁-C₄ alkyl, —CO₂—C₁-C₄ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl.

In some embodiments, Y is —NR¹R²;
R¹ and R² are each independently —H, —R³, or —C₁-C₄-alkylene-R³;
R³ is a 5-6 membered heterocycloalkyl, phenyl, —OR⁴, or —NR⁵R⁶, wherein each heterocycloalkyl or phenyl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or phenyl, wherein each alkyl or phenyl is optionally substituted with one or more halogen;
R¹¹ and R¹² are each independently —H, —C₁-C₄ alkyl, —CO₂—C₁-C₄ alkyl, phenyl, or —SO₂-phenyl; wherein each alkyl or phenyl is optionally substituted with one or more halogen;
R¹³ is a 3-15 membered heterocycloalkyl, 5- to 10-membered heteroaryl, or C₆-C₁₀ aryl; wherein each heterocycloalkyl, heteroaryl, or aryl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl.

In some embodiments, Y is —NR¹R²;
R¹ and R² are each independently —H, —R³, or —C₁-C₄-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₄ alkyl;
R⁴ is independently —H or —C₁-C₄ alkyl;
R⁵ and R⁶ are independently, at each occurrence, —H or —C₁-C₄ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or phenyl;
R¹¹ and R¹² are each independently —H, —C₁-C₄ alkyl optionally substituted with one or more halogen, —CO₂-tert-butyl, phenyl optionally substituted with one or more halogen, or —SO₂-phenyl;
R¹³ is a 3-15 membered heterocycloalkyl, 5- to 10-membered heteroaryl, or phenyl; wherein each heterocycloalkyl, heteroaryl, or phenyl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl.

In some embodiments, Y is —NR¹R²;
R¹ and R² are each independently —H, —R³, or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ and R¹² are each independently —H, —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heterocycloalkyl, heteroaryl, or phenyl is optionally substituted with one or more oxo, halogen, —OR¹⁴, or —C₁-C₄ alkyl, wherein each alkyl is optionally substituted with one or more halogen;
R¹⁴ is independently —H or —C₁-C₄ alkyl.

In some embodiments, Y is —NR¹R²;
R¹ is H;
R² is independently —H, —R³, or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is phenyl;
R¹¹ H or —C₁-C₂ alkyl;
R¹² is independently —H, —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heterocycloalkyl or heteroaryl is optionally substituted with one or more oxo, halogen, —OR¹⁴, or —C₁-C₄ alkyl, wherein each alkyl is optionally substituted with one or more halogen, and wherein each phenyl is optionally substituted with one or more halogen;
R¹⁴ is independently —H or —C₁-C₄ alkyl.

In some embodiments, Y is —NR¹R²;
R¹ is H;
R² is independently —R³ or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heteroaryl is optionally substituted with one or more oxo, halogen, or —C₁-C₄ alkyl, wherein said alkyl is optionally substituted with one or more halogen, and wherein said phenyl is optionally substituted with one or more halogen, —OR¹⁴ or —C₁-C₄ alkyl, wherein each alkyl is optionally substituted with one or more halogen;
R¹⁴ is independently —H or —C₁-C₄ alkyl.

In some embodiments, Y is —NR¹R²;
R¹ is H;
R² is independently —R³ or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl.

In some embodiments, Y is —NR¹R²;
R¹ is H;
R² is independently —R³ or —C₁-C₃-alkylene-R³;
R³ is a 1-piperidine, 4-piperidine, 1-piperazine, 4-morpholine, phenyl, —OR⁴, or —NR⁵R⁶, wherein each 1-piperidine is optionally substituted with one or more —C₁-C₄ alkyl at the 4-position, each 4-piperidine is optionally substituted with one or more —C₁-C₄ alkyl at the 1-position, and each 1-piperazine is optionally substituted with one or more —C₁-C₄ alkyl at the 4-position;
R⁴ is independently —H or —C₁-C₂ alkyl;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl.

In some embodiments, Y is —NR¹R²;
R¹ is H;
R² is independently —R³ or —C₂-C₃-alkylene-R³;
R³ is a 1-piperidine, 4-morpholine, or —NR⁵R⁶, wherein each piperidine is optionally substituted with one or more —C₁-C₂ alkyl at the 4-position;
R⁵ and R⁶ are independently, at each occurrence, —H or —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl.

In some embodiments, Y is —NR¹R²;
R¹ is H;
R² is independently —R³ or —C₂-C₃-alkylene-R³;
R³ is a 1-piperidine, 4-morpholine, or —NR⁵R⁶;
R⁵ and R⁶ are independently, at each occurrence, —C₁-C₂ alkyl;
or R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen;
R¹⁴ is independently —H or —C₁-C₂ alkyl.

In some preferred embodiments, Y is —NR¹R²;
R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₆ alkyl; and
R⁵ and R⁶ are independently, at each occurrence, —H or —C₁-C₆ alkyl.

In some embodiments, Y is —NR¹R²; R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₄ alkyl; and
R⁵ and R⁶ are independently, at each occurrence, —H or —C₁-C₄ alkyl.

In some embodiments, Y is —NR¹R²; R¹ and R² are each independently —H, —C₁-C₄ alkyl, —R³, or —C₁-C₄-alkylene-R³;
R³ is a 3-10 membered heterocycloalkyl, C₆-C₁₀ aryl, —OR⁴, or —NR⁵R⁶, wherein each heterocycloalkyl or aryl is optionally substituted with one or more —C₁-C₄ alkyl;
R⁴ is independently —H or —C₁-C₄ alkyl; and
R⁵ and R⁶ are independently, at each occurrence, —H or —C₁-C₄ alkyl.

In some embodiments, Y is —NR¹R²; R¹ and R² are each independently —H, —R³, or —C₁-C₄-alkylene-R³;
R³ is a 3-10 membered heterocycloalkyl, C₆-C₁₀ aryl, —OR⁴, or —NR⁵R⁶, wherein each heterocycloalkyl or aryl is optionally substituted with one or more —C₁-C₄ alkyl;
R⁴ is independently —H or —C₁-C₄ alkyl; and
R⁵ and R⁶ are independently, at each occurrence, —H or —C₁-C₄ alkyl.

In some embodiments, Y is —NR¹R²; R¹ and R² are each independently —H, —R³, or —C₁-C₄-alkylene-R³;
R³ is a 5-6 membered heterocycloalkyl, phenyl, —OR⁴, or —NR⁵R⁶, preferably wherein each heterocycloalkyl contains 1-3 heteroatoms selected from N and O, preferably 1-2 heteroatoms selected from N and O, and wherein each heterocycloalkyl or phenyl is optionally substituted with one or more —C₁-C₄ alkyl;
R⁴ is independently —H or —C₁-C₄ alkyl; and
R⁵ and R⁶ are independently, at each occurrence, —H or —C₁-C₄ alkyl.

In some embodiments, Y is —NR¹R²; R¹ and R² are each independently —H, —R³, or —C₁-C₄-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₄ alkyl;
R⁴ is independently —H or —C₁-C₄ alkyl; and
R⁵ and R⁶ are independently, at each occurrence, —H or —C₁-C₄ alkyl.

In some embodiments, Y is —NR¹R²; R¹ and R² are each independently —H, —R³, or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl; and
R⁵ and R⁶ are independently, at each occurrence, —H or —C₁-C₂ alkyl.

In some embodiments, Y is —NR¹R²; R¹ is H;
R² is independently —H, —R³, or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl; and
R⁵ and R⁶ are independently, at each occurrence, —H or —C₁-C₂ alkyl.

In some embodiments, Y is —NR¹R²;
R¹ is H;
R² is independently —R³ or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl; and
R⁵ and R⁶ are independently, at each occurrence, —H or —C₁-C₂ alkyl.

In some embodiments, Y is —NR¹R²; R¹ is H;
R² is independently —R³ or —C₁-C₃-alkylene-R³;
R³ is a piperidine, piperazine, morpholine, phenyl, —OR⁴, or —NR⁵R⁶, wherein each piperidine, piperazine, morpholine, or phenyl is optionally substituted with one or more —C₁-C₂ alkyl;
R⁴ is independently —H or —C₁-C₂ alkyl; and
R⁵ and R⁶ are independently, at each occurrence, —H or —C₁-C₂ alkyl.

In some embodiments, Y is —NR¹R²; R¹ is H;
R² is independently —R³ or —C₁-C₃-alkylene-R³;
R³ is a 1-piperidine, 4-piperidine, 1-piperazine, 4-morpholine, phenyl, —OR⁴, or —NR⁵R⁶, wherein each 1-piperidine is optionally substituted with one or more —C₁-C₄ alkyl at the 4-position, each 4-piperidine is optionally substituted with one or more —C₁-C₄ alkyl at the 1-position, and each 1-piperazine is optionally substituted with one or more —C₁-C₄ alkyl at the 4-position;
R⁴ is independently —H or —C₁-C₂ alkyl; and
R⁵ and R⁶ are independently, at each occurrence, —H or —C₁-C₂ alkyl.

In some embodiments, Y is —NR¹R²; R¹ is H;
R² is independently —R³ or —C₂-C₃-alkylene-R³;
R³ is a 1-piperidine, 4-morpholine, or —NR⁵R⁶, wherein each piperidine is optionally substituted with one or more —C₁-C₂ alkyl at the 4-position; and
R⁵ and R⁶ are independently, at each occurrence, —H or —C₁-C₂ alkyl.

In some embodiments, Y is —NR¹R²; R¹ is H;
R² is independently —R³ or —C₂-C₃-alkylene-R³;
R³ is a 1-piperidine, 4-morpholine, or —NR⁵R⁶; and
R⁵ and R⁶ are independently, at each occurrence, —C₁-C₂ alkyl.

In some preferred embodiments, Y is —NR¹R²;
R¹ and R², together with the nitrogen atom to which they are attached, combine to form a 3-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen; and
R¹⁴ is independently —H or —C₁-C₆ alkyl.

In some embodiments, Y is —NR¹R²;
R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen; and
R¹⁴ is independently —H or —C₁-C₆ alkyl.

In some embodiments, Y is —NR¹R²;
R¹ and R², together with the nitrogen atom to which they are attached, combine to form a 4-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or —C₆-C₁₀ aryl, wherein each alkyl or aryl is optionally substituted with one or more halogen;
R¹¹ and R¹² are each independently —H, —C₁-C₄ alkyl, —CO₂—C₁-C₄ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₄ alkyl, wherein each alkyl is optionally substituted with one or more halogen; and
R¹⁴ is independently —H or —C₁-C₄ alkyl.

In some embodiments, Y is —NR¹R²;
R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or phenyl, wherein each alkyl or phenyl is optionally substituted with one or more halogen;
R¹¹ and R¹² are each independently —H, —C₁-C₄ alkyl, —CO₂—C₁-C₄ alkyl, phenyl, or —SO₂-phenyl; wherein each alkyl or phenyl is optionally substituted with one or more halogen;
R¹³ is a 3-15 membered heterocycloalkyl, 5- to 10-membered heteroaryl, or C₆-C₁₀ aryl; wherein each heterocycloalkyl, heteroaryl, or aryl is optionally substituted with one or more oxo, halogen, —OR¹⁴, or —C₁-C₄ alkyl, wherein each alkyl is optionally substituted with one or more halogen; and
R¹⁴ is independently —H or —C₁-C₄ alkyl.

In some embodiments, Y is —NR¹R²;
R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₄ alkyl, or phenyl;
R¹¹ and R¹² are each independently —H, —C₁-C₄ alkyl optionally substituted with one or more halogen, —CO₂-tert-butyl, phenyl optionally substituted with one or more halogen, or —SO₂-phenyl;
R¹³ is a 3-15 membered heterocycloalkyl, 5- to 10-membered heteroaryl, or phenyl; wherein each heterocycloalkyl, heteroaryl, or phenyl is optionally substituted with one or more oxo, halogen, —OR¹⁴, or —C₁-C₄ alkyl, wherein each alkyl is optionally substituted with one or more halogen; and
R¹⁴ is independently —H or —C₁-C₄ alkyl.

In some embodiments, Y is —NR¹R²;
R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ and R¹² are each independently —H, —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heterocycloalkyl, heteroaryl, or phenyl is optionally substituted with one or more oxo, halogen, —OR¹⁴, or —C₁-C₄ alkyl, wherein each alkyl is optionally substituted with one or more halogen; and
R¹⁴ is independently —H or —C₁-C₄ alkyl.

In some embodiments, Y is —NR¹R²;
R¹ and R², together with the nitrogen atom to which they are attached, combine to form a 4-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H or phenyl; R¹¹ H or —C₁-C₂ alkyl; R¹² is independently —H, —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; wherein each heterocycloalkyl or heteroaryl is optionally substituted with one or more oxo, halogen, —OR¹⁴, or —C₁-C₄ alkyl, wherein each alkyl is optionally substituted with one or more halogen, and wherein each phenyl is optionally substituted with one or more halogen; and
R¹⁴ is independently —H or —C₁-C₄ alkyl.

In some embodiments, Y is —NR¹R²;
R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl; R¹¹ H or —C₁-C₂ alkyl; R¹² is independently —C₁-C₂ alkyl optionally substituted with one or more halogen, —CO₂-tert-butyl, phenyl optionally substituted with one or more halogen, or —SO₂-phenyl;
R¹³ is a 6- to 9-membered heterocycloalkyl, 5- to 9-membered heteroaryl, or phenyl; preferably wherein each heterocycle or heteroaryl contains 1-3 heteroatoms selected from N and O, preferably 1-2 heteroatoms selected from N and O, and wherein each heteroaryl is optionally substituted with one or more oxo, halogen, or —C₁-C₄ alkyl, wherein said alkyl is optionally substituted with one or more halogen, and wherein said phenyl is optionally substituted with one or more halogen, —OR¹⁴ or —C₁-C₄ alkyl, wherein each alkyl is optionally substituted with one or more halogen; and
R¹⁴ is independently —H or —C₁-C₄ alkyl.

In some embodiments, Y is —NR¹R²;
R¹ and R², together with the nitrogen atom to which they are attached, combine to form a 4-13-membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, or phenyl;
R¹¹ H or —C₁-C₂ alkyl;
R¹² is independently —C₁-C₂ alkyl, —CO₂-tert-butyl, phenyl optionally substituted with one or more halogen, or —SO₂-phenyl;
R¹³ is a heterocycloalkyl selected from piperidine, 5,6,7,8-tetrahydroimidazo[1,2-a]pyrazine, piperazine, and morpholine; a heteroaryl selected from 1,3-dihydro-2H-benzimidazol-2-one, imidazole, pyrrole, pyrazole, pyrazine, pyridine, and indole; or phenyl; wherein each heteroaryl is optionally substituted with one or more halogen or —C₁-C₂ alkyl, and wherein each phenyl is optionally substituted with one or more halogen, —OR¹⁴, or —C₁-C₄ alkyl, wherein each alkyl is optionally substituted with one or more halogen; and
R¹⁴ is independently —H or —C₁-C₂ alkyl.

In some preferred embodiments, Y is —NR¹R²;
R¹ and R², together with the nitrogen atom to which they are attached, combine to form a piperidine, piperazine, or morpholine ring, wherein said piperidine, piperazine, or morpholine is optionally substituted with one or more —C₁-C₆ alkyl, halogen, —OR¹⁰, —NR¹¹R¹², oxo, R¹³, or —C₁-C₆ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen; and
R¹⁴ is independently —H or —C₁-C₆ alkyl.

In some embodiments, Y is —NR¹R²; R¹ and R², together with the nitrogen atom to which they are attached, combine to form a piperidine, piperazine, or morpholine ring, wherein said piperidine or piperazine is optionally substituted with one or more —C₁-C₄ alkyl, —OR¹⁰, —NR¹¹R¹², —R¹³, or —C₁-C₄ alkylene-R¹³; wherein each alkyl or alkylene is optionally substituted with one or more —OH;
R¹⁰ is independently —H, —C₁-C₄ alkyl, or phenyl, wherein each alkyl or phenyl is optionally substituted with one or more halogen;
R¹¹ and R¹² are each independently —H, —C₁-C₄ alkyl wherein said alkyl is optionally substituted by one or more halogen, —CO₂-tert-butyl, —SO₂-phenyl, or phenyl wherein said phenyl is optionally substituted with one or more halogen;
R¹³ is a C₃-C₆ cycloalkyl, 3-15 membered heterocycloalkyl, 5- to 10-membered heteroaryl, or phenyl; wherein each cycloalkyl, heterocycloalkyl, heteroaryl, or phenyl is optionally substituted with one or more oxo, halogen, —OR¹⁴, or —C₁-C₆ alkyl, wherein each alkyl is optionally substituted with one or more halogen; and
R¹⁴ is independently —H or —C₁-C₄ alkyl.

In some embodiments, Y is —NR¹R²;
R¹ and R², together with the nitrogen atom to which they are attached, combine to form a piperidine, piperazine, or morpholine ring, wherein said piperazine is optionally substituted by one or more phenyl or —C₁-C₄ alkyl, and wherein said piperidine is optionally substituted with one or more —OR¹⁰, —NR¹¹R¹², —R¹³, or —C₁-C₄ alkylene-R¹³; wherein each alkylene is optionally substituted with one or more —OH;
R¹⁰ is independently —H or phenyl, wherein each phenyl is optionally substituted with one or more halogen;
R¹¹ and R¹² are each independently —H, —C₁-C₂ alkyl wherein said alkyl is optionally substituted by one or more halogen, —CO₂-tert-butyl, —SO₂-phenyl, or phenyl wherein said phenyl is optionally substituted with one or more halogen;
R¹³ is a 6- to 9-membered heterocycloalkyl, 5- to 9-membered heteroaryl, or phenyl; preferably wherein each heterocycle or heteroaryl contains 1-3 heteroatoms selected from N and O, preferably 1-2 heteroatoms selected from N and O, and wherein each heteroaryl is optionally substituted with one or more oxo, halogen, or —C₁-C₄ alkyl, wherein said alkyl is optionally substituted with one or more halogen, and wherein said phenyl is optionally substituted with one or more halogen, —OR¹⁴ or —C₁-C₄ alkyl, wherein each alkyl is optionally substituted with one or more halogen; and
R¹⁴ is independently —H or —C₁-C₂ alkyl.

In some embodiments, Y is —NR¹R²;
R¹ and R², together with the nitrogen atom to which they are attached, combine to form a piperidine, piperazine, or morpholine ring, wherein said piperazine is optionally substituted by one or more phenyl or —C₁-C₄ alkyl, and wherein said piperidine is optionally substituted with one or more —OR¹⁰, —NR¹¹R¹², —R¹³, or —C₁-C₄ alkylene-R¹³; wherein each alkylene is optionally substituted with one or more —OH;
R¹⁰ is independently —H or phenyl;
R¹¹ is H or —C₁-C₂ alkyl;
R¹² is independently —H, —C₁-C₂ alkyl, —CO₂-tert-butyl, —SO₂-phenyl;
R¹³ is a heterocycloalkyl selected from piperidine and 5,6,7,8-tetrahydroimidazo[1,2-a]pyrazine; a heteroaryl selected from 1,3-dihydro-2H-benzimidazol-2-one and imidazole; or phenyl; wherein each heteroaryl is optionally substituted with one or more halogen or —C₁-C₂ alkyl, and wherein each phenyl is optionally substituted with one or more halogen, —OR¹⁴, or —C₁-C₂ alkyl, wherein each alkyl is optionally substituted with one or more halogen; and
R¹⁴ is independently —H or —C₁-C₂ alkyl.

In some embodiments, Y is —NR¹R²;
R¹ and R², together with the nitrogen atom to which they are attached, combine to form a piperidine, piperazine, or morpholine ring, wherein said piperazine is optionally substituted at the 4-position by phenyl or —C₁-C₄ alkyl, and wherein said piperidine is optionally substituted at the 4-position with one or more —OR¹⁰, —NR¹¹R¹², —R¹³, or —C₁-C₃ alkylene-R¹³; wherein each alkylene is optionally substituted with one or more —OH;
R¹⁰ is independently —H or phenyl;
R¹¹ is H or —C₁-C₂ alkyl;
R¹² is independently —H, —C₁-C₂ alkyl, —CO₂-tert-butyl, —SO₂-phenyl;
R¹³ is a heterocycloalkyl selected from piperidine and 5,6,7,8-tetrahydroimidazo[1,2-a]pyrazine; a heteroaryl selected from 1,3-dihydro-2H-benzimidazol-2-one and imidazole; or phenyl; wherein each heteroaryl is optionally substituted with one or more halogen or —C₁-C₂ alkyl, and wherein each phenyl is optionally substituted with one or more halogen, —OR¹⁴, or —C₁-C₂ alkyl, wherein each alkyl is optionally substituted with one or more halogen; and
R¹⁴ is independently —H or —C₁-C₂ alkyl.

In some embodiments, Y is —NR¹R²;
R¹ and R², together with the nitrogen atom to which they are attached, combine to form a piperidine, piperazine, or morpholine ring, wherein said piperazine is optionally substituted at the 4-position by phenyl or —C₁-C₄ alkyl, and wherein said piperidine is optionally substituted at the 4-position with one or more —OR¹⁰, —NR¹¹R¹², —R¹³, or —C₁-C₃ alkylene-R¹³; wherein each alkylene is optionally substituted with one or more —OH;
R¹⁰ is independently —H or phenyl;
R¹¹ is H or —C₁-C₂ alkyl;
R¹² is independently —H, —C₁-C₂ alkyl, —CO₂-tert-butyl, —SO₂-phenyl;
R¹³ is a heterocycloalkyl selected from piperidine and 5,6,7,8-tetrahydroimidazo[1,2-a]pyrazine; a heteroaryl selected from 1,3-dihydro-2H-benzimidazol-2-one and imidazole; or phenyl; wherein each heteroaryl is optionally substituted with one or more halogen or —C₁-C₂ alkyl, and wherein each phenyl is optionally substituted with one or more —OR¹⁴ or —C₁-C₂ alkyl, wherein each alkyl is optionally substituted with one or more halogen; and
R¹⁴ is independently —H or —C₁-C₂ alkyl.

In some preferred embodiments, Y is —NR¹R²;
R¹ and R², together with the nitrogen atom to which they are attached, combine to form an azetidine ring, wherein said azetidine ring is optionally substituted with one or more heteroaryl or —NR¹¹R¹²; and
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each —C₁-C₆ alkyl and —C₆-C₁₀ aryl is optionally substituted with one or more halogen.

In some embodiments, wherein Y is —NR¹R²;
R¹ and R², together with the nitrogen atom to which they are attached, combine to form an azetidine ring, wherein said azetidine ring is optionally substituted with one or more heteroaryl or —NR¹¹R¹²; and
R¹¹ and R¹² are each independently —H, —C₁-C₆ alkyl, or phenyl wherein each phenyl is optionally substituted with one or more halogen.

In some embodiments, wherein Y is —NR¹R²; R¹ and R², together with the nitrogen atom to which they are attached, combine to form an azetidine ring, wherein said azetidine ring is optionally substituted with one or more pyrrole or —NR¹¹R¹²; and
R¹¹ and R¹² are each independently —H or phenyl; wherein each phenyl is optionally substituted with one or more fluorine.

In some embodiments, wherein Y is —NR¹R²;
R¹ and R², together with the nitrogen atom to which they are attached, combine to form an azetidine ring, wherein said azetidine ring is optionally substituted with one or more pyrrole or —NR¹¹R¹²;
R¹¹ is —H; and
R¹² is independently —H or phenyl; wherein said phenyl is optionally substituted with one or more fluorine.

In some preferred embodiments, Y is —NR¹R²;
R¹ and R², together with the nitrogen atom to which they are attached, combine to form a heterocycle selected from 1,3,8-triaza-spiro[4.5]decane, decahydro-[1,6]naphthyridine, 2,3,4,5-tetrahydro-1H-pyrido[4,3-b]indole, or [1,4]diazepane; wherein said heterocycle is optionally substituted with one or more oxo, —C₁-C₆ alkylene R¹³, or R¹³; and
R¹³ is a C₆-C₁₀ aryl; wherein said aryl is optionally substituted with one or more halogen.

In some embodiments, Y is —NR¹R² and
R¹ and R², together with the nitrogen atom to which they are attached, combine to form a heterocycle selected from 1,3,8-triaza-spiro[4.5]decane, decahydro-[1,6]naphthyridine, 2,3,4,5-tetrahydro-1H-pyrido[4,3-b]indole, or [1,4]diazepane; wherein said heterocycle is optionally substituted with one or more oxo, —C₁-C₃ alkylene R¹³, or R¹³; and
R¹³ is a phenyl; wherein said phenyl is optionally substituted with one or more halogen.

In some embodiments, Y is —NR¹R²;
R¹ and R², together with the nitrogen atom to which they are attached, combine to form a heterocycle selected from 1,3,8-triaza-spiro[4.5]decane, decahydro-[1,6]naphthyridine, 2,3,4,5-tetrahydro-1H-pyrido[4,3-b]indole, or [1,4]diazepane; wherein said 1,3,8-triaza-spiro[4.5]decane, decahydro-[1,6]naphthyridine, or [1,4]diazepane is optionally substituted with one or more oxo, —C₁-C₂ alkylene R¹³, or R¹³; and
R¹³ is a phenyl; wherein said phenyl is optionally substituted with one or more fluorine.

In some preferred embodiments, Y is [NR⁵¹R⁵²R⁵³]⁺X⁻.

In some preferred embodiments, Y is [NR⁵¹R⁵²R⁵³]⁺X⁻, wherein R⁵¹, R⁵² and R⁵³ are each independently selected from —H and —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently F⁻, Cl⁻, Br⁻, I⁻, F₃CSO₃⁻, or H₃CC₆H₄SO₃⁻.

In some embodiments, Y is [NR⁵¹R⁵²R⁵³]⁺X⁻, wherein R⁵¹, R⁵² and R⁵³ are each independently selected from —H and —C₁-C₄ alkyl;
or R⁵¹, R¹² and R⁵³, together with the nitrogen atom to which they are attached, combine to form a 5 to 10-membered ring heteroaryl, preferably wherein said heteroaryl contains 1-3 heteroatoms selected from N and O, preferably 1-2 heteroatoms selected from N and O, and wherein said heteroaryl is optionally substituted with one or more halogen, —C₁-C₄ alkyl, —OC₁-C₄ alkyl, or —N(C₁-C₄ alkyl)₂; and
X⁻ is independently F⁻, Cl⁻, Br⁻, I⁻, F₃CSO₃⁻, or H₃CC₆H₄SO₃⁻.

In some preferred embodiments, Y is [NR⁵¹R⁵²R⁵³]⁺X⁻, wherein R⁵¹, R⁵² and R⁵³ are each independently selected from —H and —C₁-C₆ alkyl;
or R⁵¹, R⁵² and R⁵³, together with the nitrogen atom to which they are attached, combine to form a 6 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently F⁻, Cl⁻, Br⁻, I⁻, F₃CSO₃⁻, or H₃CC₆H₄SO₃⁻.

In some embodiments, Y is [NR⁵¹R⁵²R⁵³]⁺X⁻, wherein R⁵¹, R⁵² and R⁵³ are each independently selected from —H and —C₁-C₄ alkyl;
or R⁵¹, R⁵² and R⁵³, together with the nitrogen atom to which they are attached, combine to form a 6 to 10-membered ring heteroaryl, preferably wherein said heteroaryl contains 1-3 heteroatoms selected from N and O, preferably 1-2 heteroatoms selected from N and O, and wherein said heteroaryl is optionally substituted with one or more halogen, —C₁-C₄ alkyl, —OC₁-C₄ alkyl, or —N(C₁-C₄ alkyl)₂; and
X⁻ is independently F⁻, Cl⁻, Br⁻, I⁻, F₃CSO₃⁻, or H₃CC₆H₄SO₃⁻.

In some embodiments, Y is [NR⁵R⁵²R⁵³]⁺X⁻, wherein R⁵¹, R⁵² and R⁵³ are each independently selected from —H and —C₁-C₄ alkyl;
or R⁵¹, R⁵² and R⁵³, together with the nitrogen atom to which they are attached, combine to form a 1-pyridinium, wherein said 1-pyridinium is optionally substituted with one or more halogen, —C₁-C₄ alkyl, —OC₁-C₄ alkyl, or —N(C₁-C₄ alkyl)₂; and
X⁻ is independently F⁻, Cl⁻, Br⁻, I⁻, F₃CSO₃⁻, or H₃CC₆H₄SO₃⁻.

In some embodiments, Y is [NR⁵R⁵²R⁵³]⁺X⁻, wherein R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more halogen, —C₁-C₂ alkyl, —OC₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, Y is [NR⁵R⁵²R⁵³]⁺X⁻, wherein R⁵¹, R⁵² and R⁵³ are each independently selected from —H and —C₁-C₂ alkyl;
or R⁵¹, R⁵² and R⁵³, together with the nitrogen atom to which they are attached, combine to form a 1-pyridinium, wherein said 1-pyridinium is optionally substituted with one or more —C₁-C₂ alkyl, or —N(C₁-C₂ alkyl)₂; and
X⁻ is independently F⁻, Cl⁻, Br⁻, I⁻, F₃CSO₃⁻, or H₃CC₆H₄SO₃⁻.

In some embodiments, X⁻ is independently a pharmaceutically acceptable anion. In some embodiments, X⁻ is independently F⁻, Cl⁻, Br⁻, or I⁻. In some embodiments, X⁻ is independently Cl⁻, or Br⁻. In some embodiments, X⁻ is Br.

In one or more embodiments, Y of a compound of the present invention can form one of the structures selected from Table 1, below:

TABLE 1
Exemplary Embodiments of Y

| Embodiment | Structure |
|---|---|
| 1 | 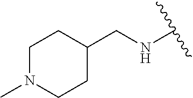 [SMILES: *NCC1CCN(C)CC1] |
| 2 | 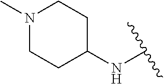 [SMILES: *NC1CCN(C)CC1] |
| 3 | 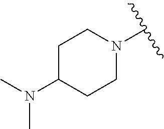 [SMILES: *N1CCC(N(C)C)CC1] |
| 4 | 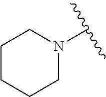 [SMILES: *N1CCCCC1] |
| 5 | 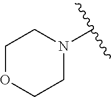 [SMILES: *N1CCOCC1] |
| 6 | 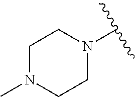 [SMILES: *N1CCN(C)CC1] |
| 7 | 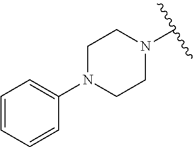 [SMILES: *N1CCN(c2ccccc2)CC1] |
| 8 | 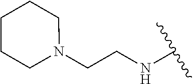 [SMILES: *NCCN1CCCCC1] |
| 9 | 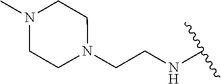 [SMILES: *NCCN1CCN(C)CC1] |
| 10 | 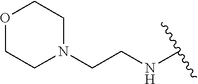 [SMILES: *NCCN1CCOCC1] |
| 11 | 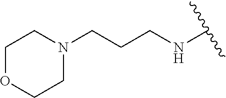 [SMILES: *NCCCN1CCOCC1] |
| 12 | 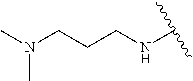 [SMILES: *NCCCN(C)C] |
| 13 | 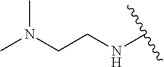 [SMILES: *NCCN(C)C] |
| 14 | 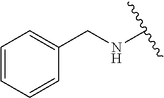 [SMILES: *NCc1ccccc1] |
| 15 | 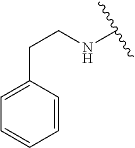 [SMILES: *NCCc1ccccc1] |
| 16 | 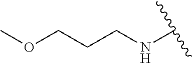 [SMILES: *NCCCOC] |
| 17 | 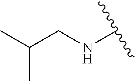 [SMILES: *NCC(C)C] |
| 18 | 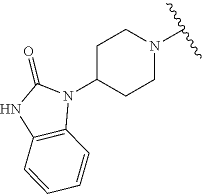 [SMILES: *N1CCC(n2c(=O)[nH]c3ccccc32)CC1] |
| 19 | 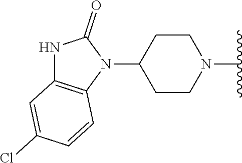 [SMILES: *N1CCC(n2c(=O)[nH]c3cc(Cl)ccc32)CC1] |
| 20 | 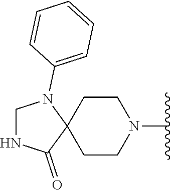 [SMILES: *N1CCC2(CC1)C(=O)NCN2c1ccccc1] |
| 21 | 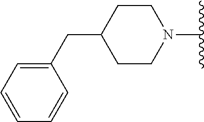 [SMILES: *N1CCC(Cc2ccccc2)CC1] |
| 22 | 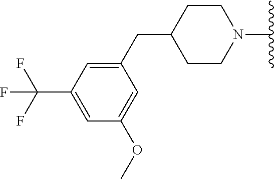 [SMILES: *N1CCC(Cc2cc(OC)cc(C(F)(F)F)c2)CC1] |
| 23 | 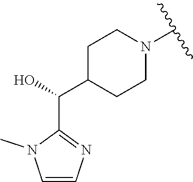 [SMILES: *N1CCC([C@@H](O)c2nccn2C)CC1] |
| 24 | 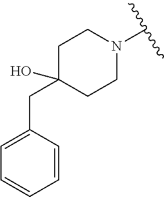 [SMILES: *N1CCC(O)(Cc2ccccc2)CC1] |
| 25 | 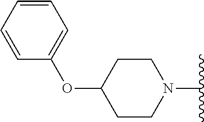 [SMILES: *N1CCC(Oc2ccccc2)CC1] |
| 26 | 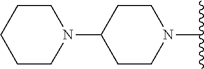 [SMILES: *N1CCC(N2CCCCC2)CC1] |
| 27 | 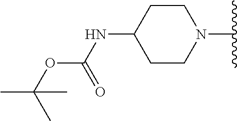 [SMILES: *N1CCC(NC(=O)OC(C)(C)C)CC1] |
| 28 | 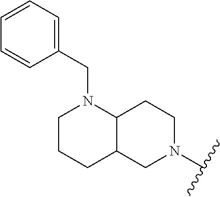 [SMILES: *N1CCC2C(CCCN2Cc2ccccc2)C1] |
| 29 | 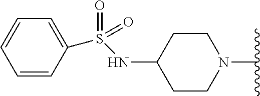 [SMILES: *N1CCC(NS(=O)(=O)c2ccccc2)CC1] |
| 30 | 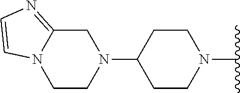 [SMILES: *N1CCC(N2CCn3ccnc3C2)CC1] |
| 31 | 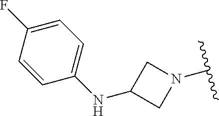 [SMILES: *N1CC(Nc2ccc(F)cc2)C1] |
| 32 | 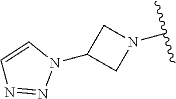 [SMILES: *N1CC(n2ccnn2)C1] |
| 33 | 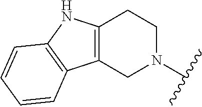 [SMILES: *N1CCc2[nH]c3ccccc3c2C1] |
| 34 | 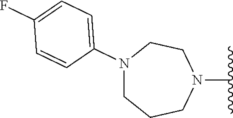 [SMILES: *N1CCCN(c2ccc(F)cc2)CC1] |
| 35 | 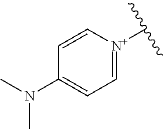 [SMILES: *[n+]1ccc(N(C)C)cc1] |
| 36 | 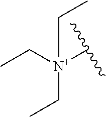 [SMILES: *[N+](CC)(CC)CC] |
| 37 | 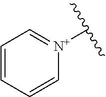 [SMILES: *[n+]1ccccc1] |
| 38 | 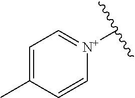 [SMILES: *[n+]1ccc(C)cc1] |
| 39 | 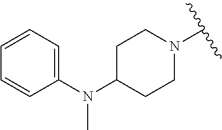 [SMILES: *N1CCC(N(C)c2ccccc2)CC1] |
| 40 | 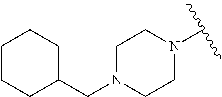 [SMILES: *N1CCN(CC2CCCCC2)CC1] |

In one or more embodiments of any of the above aspects, the compound of Formula I is selected from Table 2, below:

TABLE 2
Exemplary Compounds (Cpd) of the Invention

| Cpd | Structure |
|---|---|
| 1 | 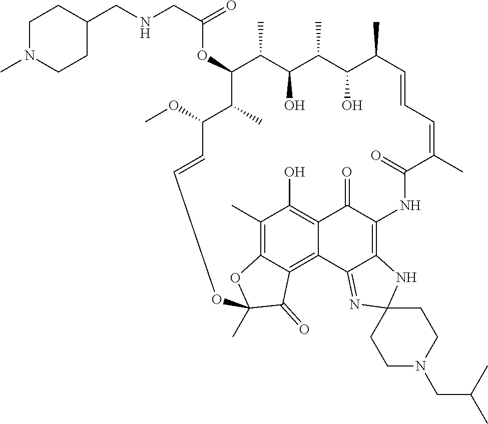 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCC2CCN(C)CC2)[C@@H]1C)C4=O] |
| 2 | 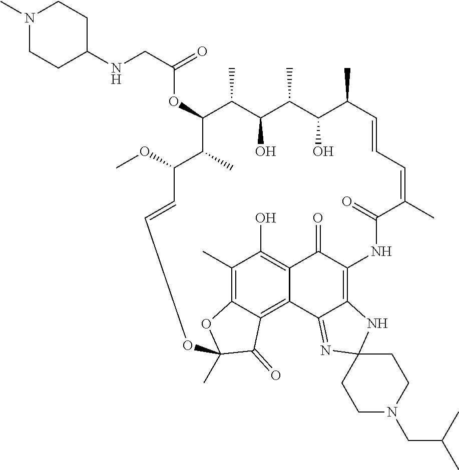 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNC2CCN(C)CC2)[C@@H]1C)C4=O] |
| 3 | 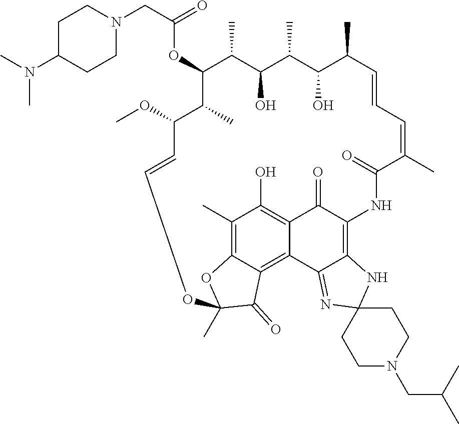 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)C)CC2)[C@@H]1C)C4=O] |
| 4 | 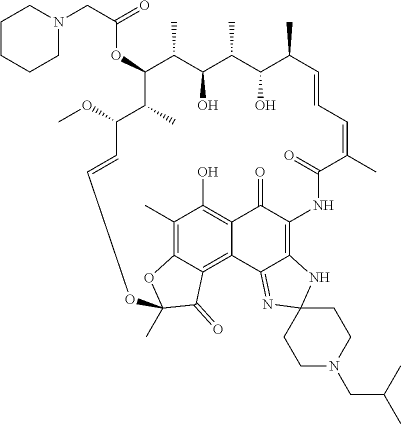 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCCC2)[C@@H]1C)C4=O] |
| 5 | 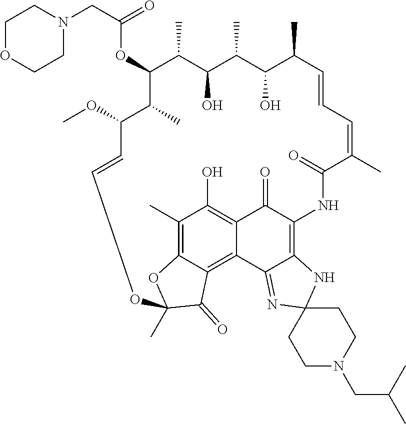 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCOCC2)[C@@H]1C)C4=O] |
| 6 | 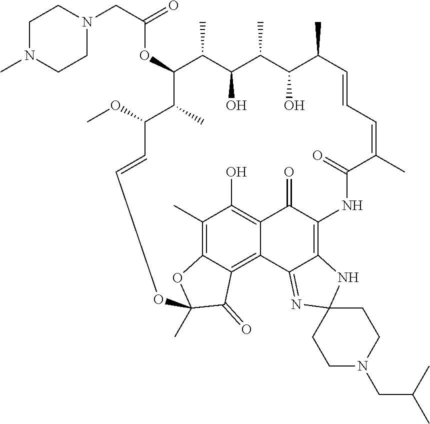 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(C)CC2)[C@@H]1C)C4=O] |
| 7 | 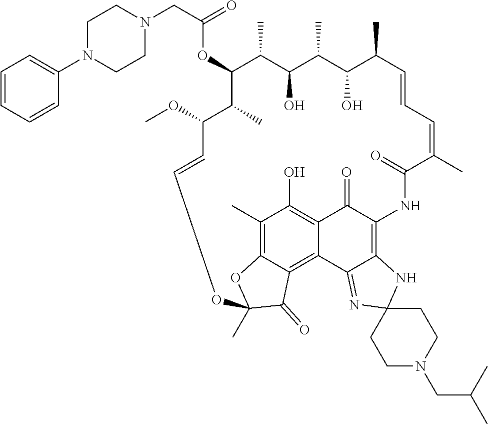 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(c3ccccc3)CC2)[C@@H]1C)C4=O] |
| 8 | 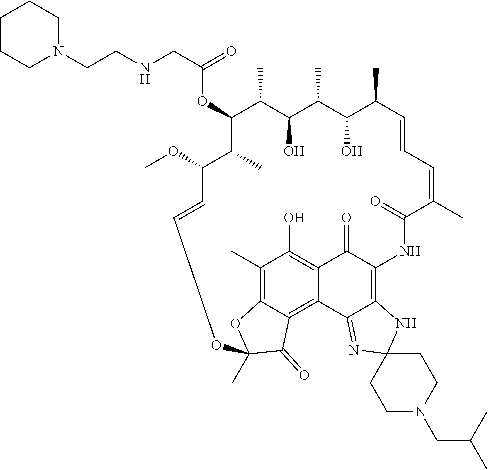 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCCCC2)[C@@H]1C)C4=O] |
| 9 | 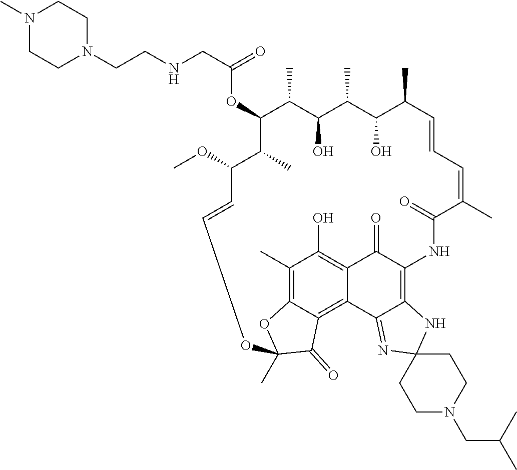 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCN(C)CC2)[C@@H]1C)C4=O] |
| 10 | 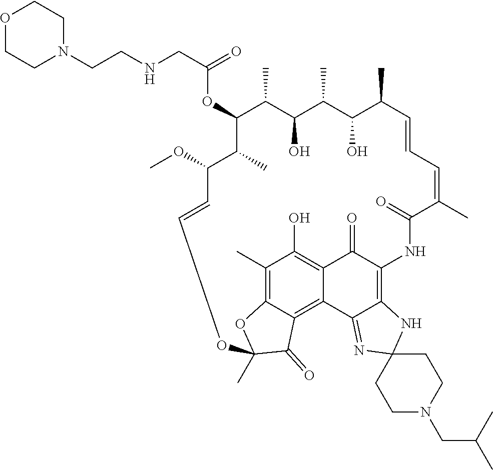 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCOCC2)[C@@H]1C)C4=O] |
| 11 | 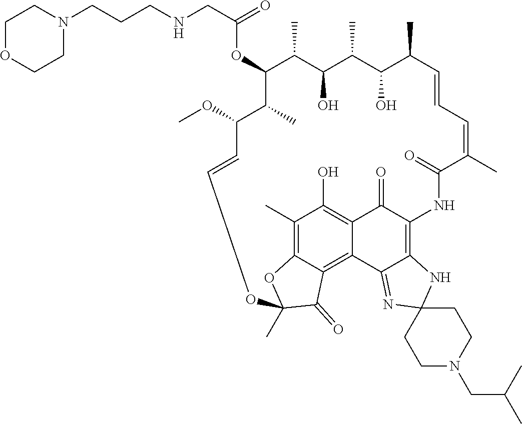 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN2CCOCC2)[C@@H]1C)C4=O] |
| 12 | 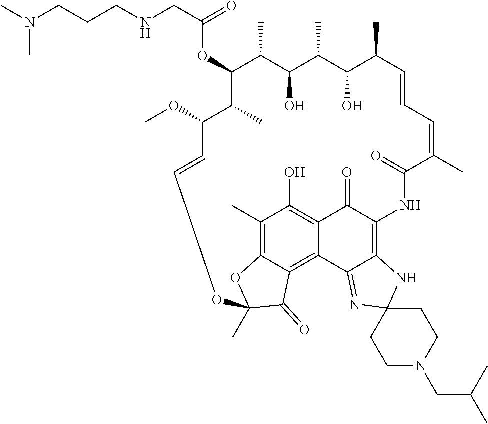 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN(C)C)[C@@H]1C)C4=O] |
| 13 | 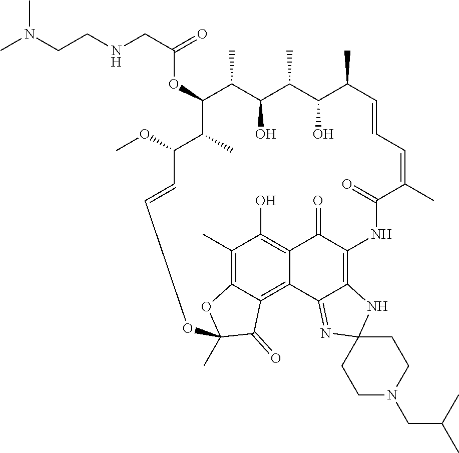 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN(C)C)[C@@H]1C)C4=O] |
| 14 | 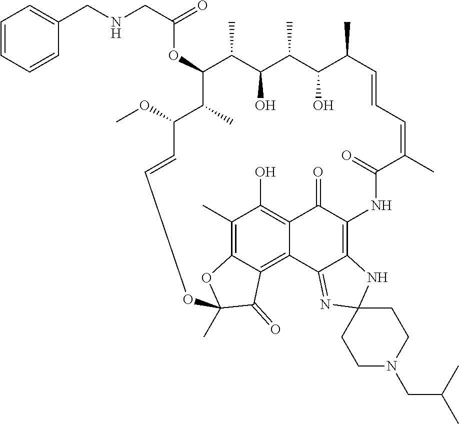 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCc2ccccc2)[C@@H]1C)C4=O] |
| 15 | 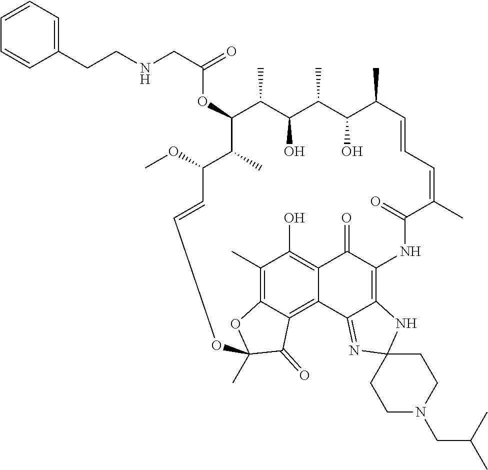 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCc2ccccc2)[C@@H]1C)C4=O] |
| 16 | 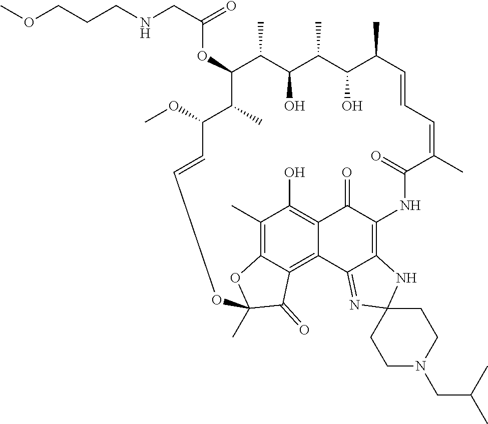 [SMILES: COCCCNCC(=O)O[C@H]1[C@H](C)[C@H](O)[C@H](C)[C@@H](O)[C@@H](C)/C=C/C=C(/C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O] |
| 17 | 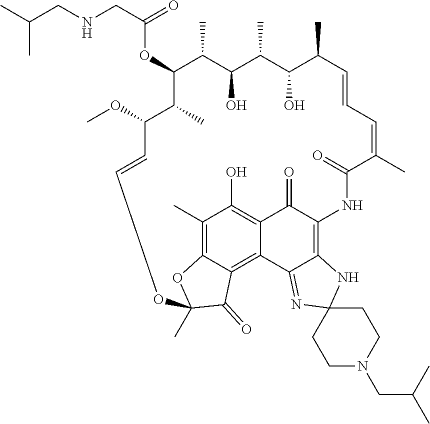 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCC(C)C)[C@@H]1C)C4=O] |
| 18 | 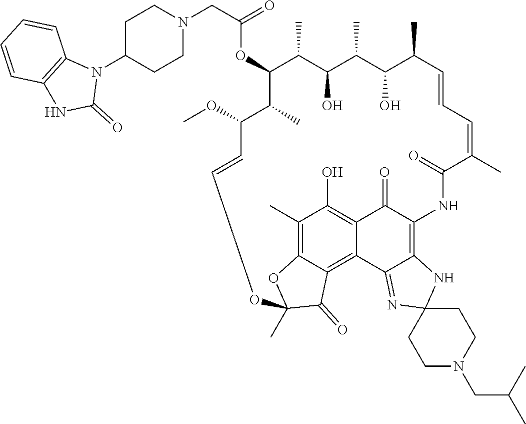 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5ccccc53)CC2)[C@@H]1C)C4=O] |
| 19 | 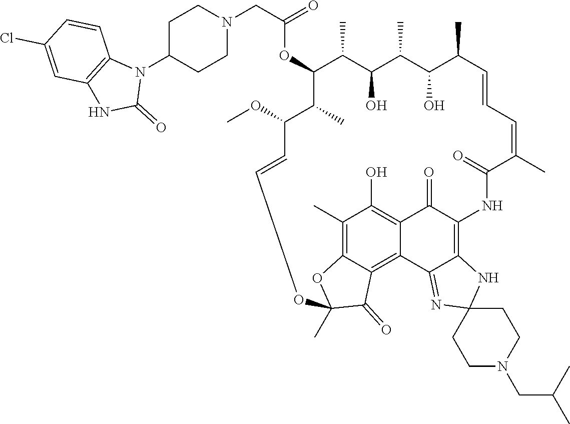 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5cc(Cl)ccc53)CC2)[C@@H]1C)C4=O] |
| 20 | 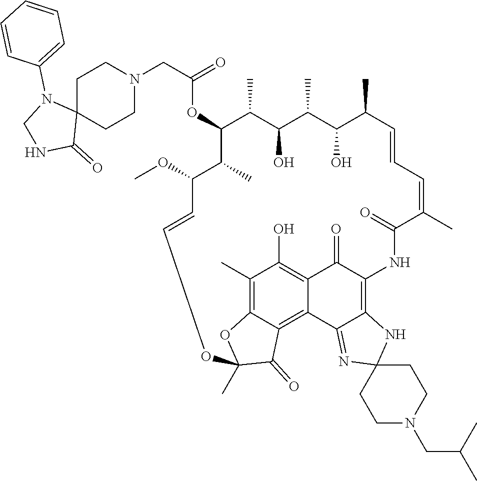 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3(CC2)C(=O)NCN3c2ccccc2)[C@@H]1C)C4=O] |
| 21 | 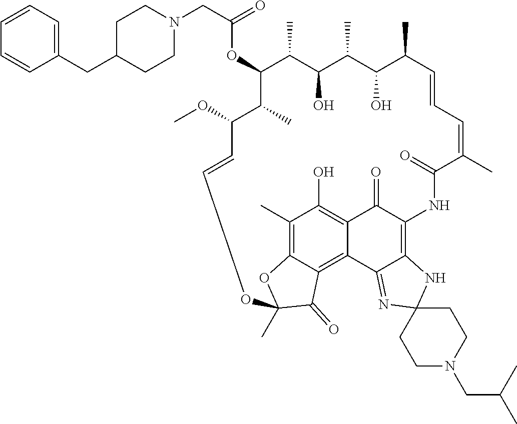 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Cc3ccccc3)CC2)[C@@H]1C)C4=O] |
| 22 | 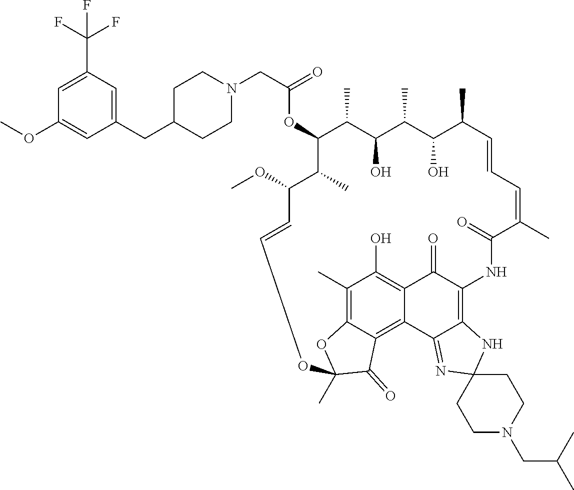 [SMILES: COc1cc(CC2CCN(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)CC2)cc(C(F)(F)F)c1] |
| 23 | 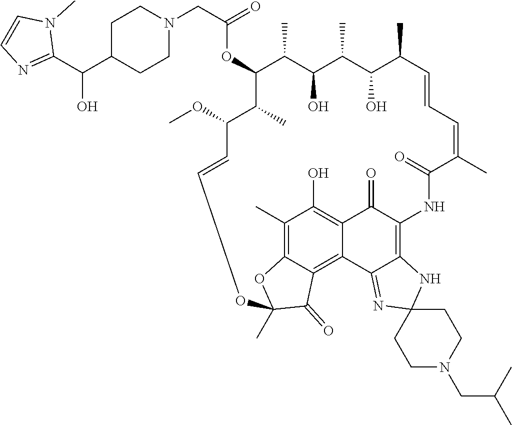 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(C(O)c3nccn3C)CC2)[C@@H]1C)C4=O] |
| 24 | 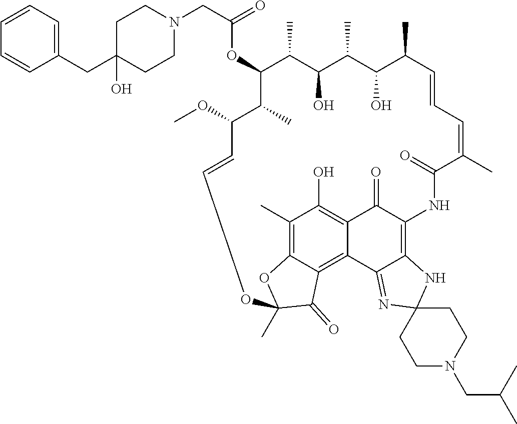 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(O)(Cc3ccccc3)CC2)[C@@H]1C)C4=O] |
| 25 | 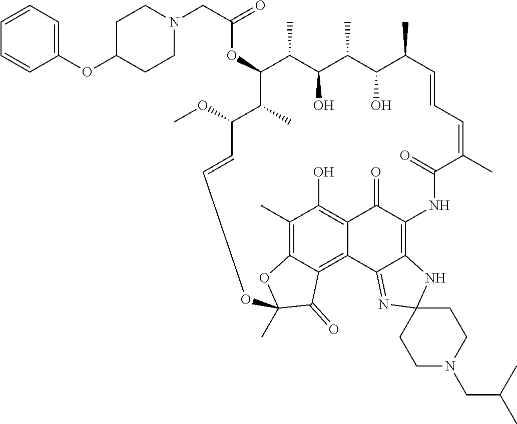 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Oc3ccccc3)CC2)[C@@H]1C)C4=O] |
| 26 | 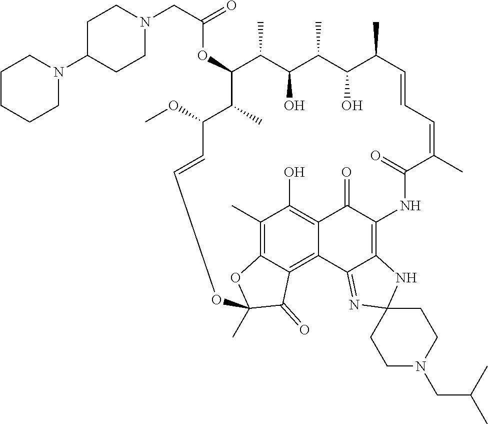 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCCCC3)CC2)[C@@H]1C)C4=O] |
| 27 | 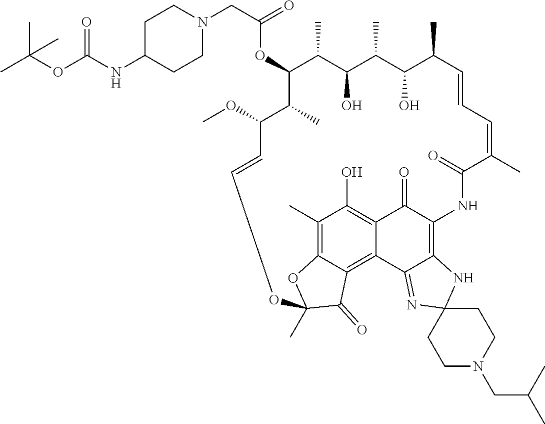 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NC(=O)OC(C)(C)C)CC2)[C@@H]1C)C4=O] |
| 28 | 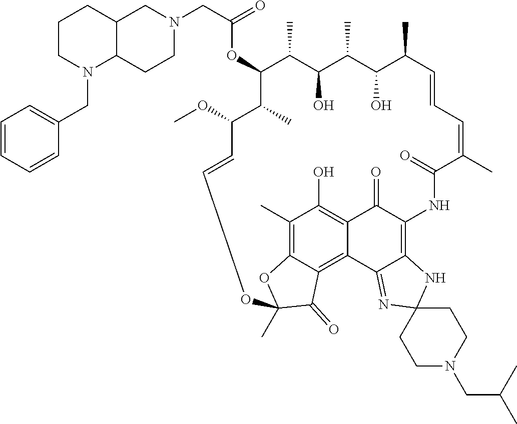 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3C(CCCN3Cc3ccccc3)C2)[C@@H]1C)C4=O] |
| 29 | 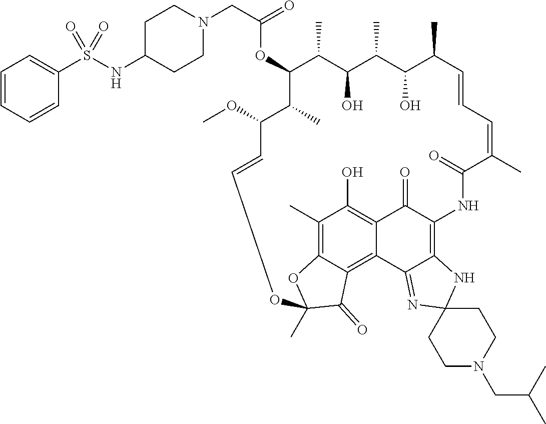 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NS(=O)(=O)c3ccccc3)CC2)[C@@H]1C)C4=O] |
| 30 | 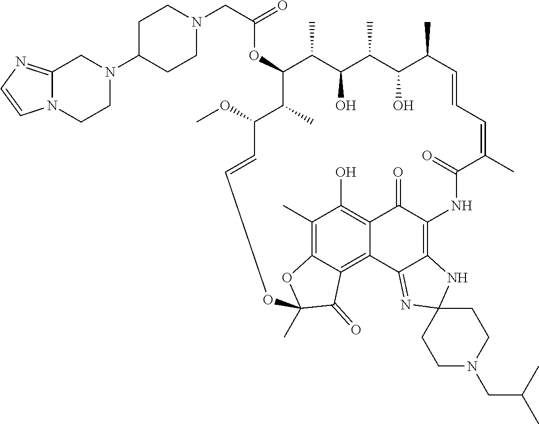 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCn5ccnc5C3)CC2)[C@@H]1C)C4=O] |
| 31 | 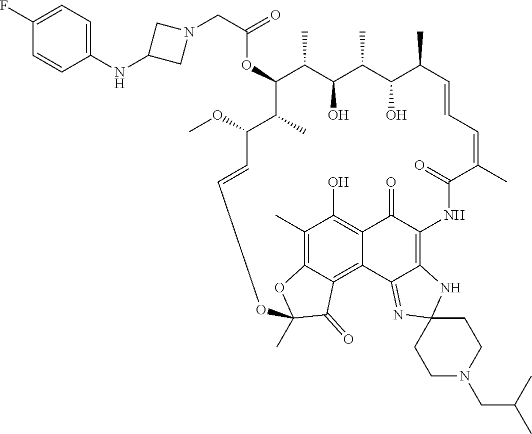 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CC(Nc3ccc(F)cc3)C2)[C@@H]1C)C4=O] |
| 32 | 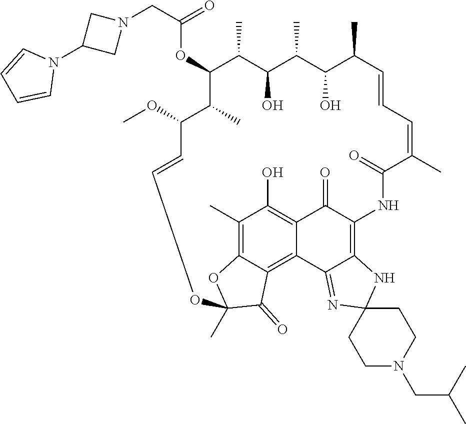 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CC(n3cccc3)C2)[C@@H]1C)C4=O] |
| 33 | 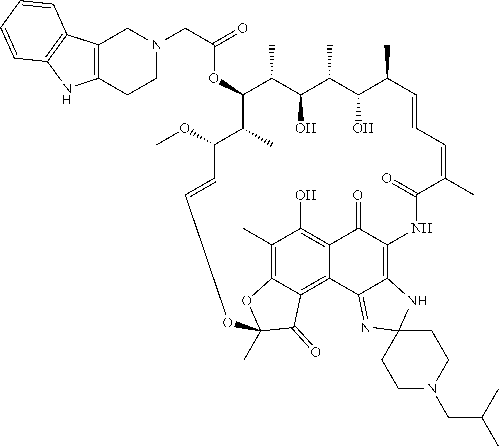 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCc3[nH]c5ccccc5c3C2)[C@@H]1C)C4=O] |
| 34 | 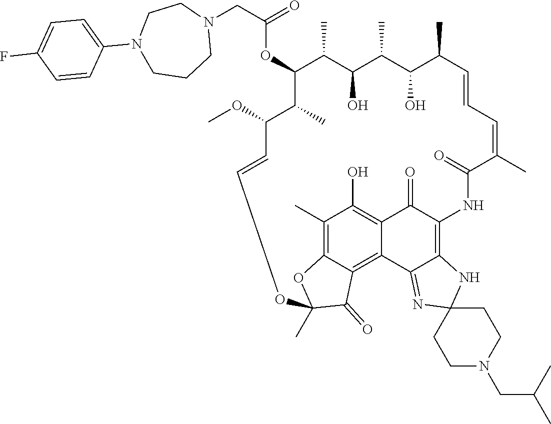 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCN(c3ccc(F)cc3)CC2)[C@@H]1C)C4=O] |
| 35 | 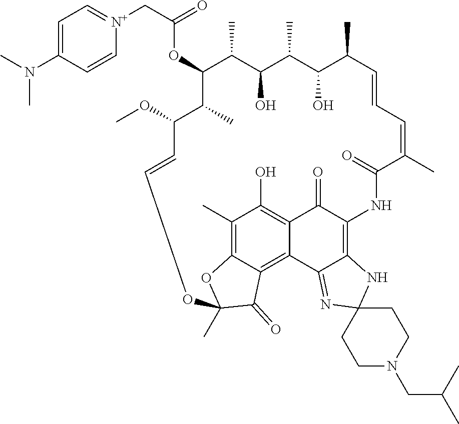 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccc(N(C)C)cc2)[C@@H]1C)C4=O] |
| 36 | 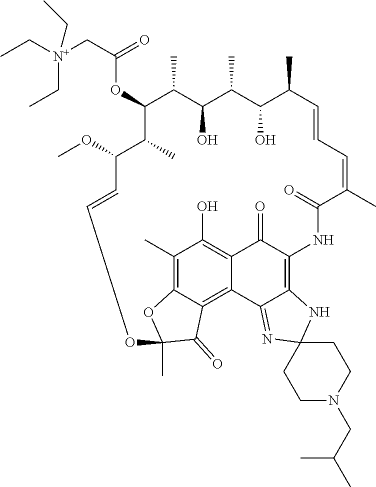 [SMILES: CC[N+](CC)(CC)CC(=O)O[C@H]1[C@H](C)[C@H](O)[C@H](C)[C@@H](O)[C@@H](C)/C=C/C=C(/C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O] |
| 37 | 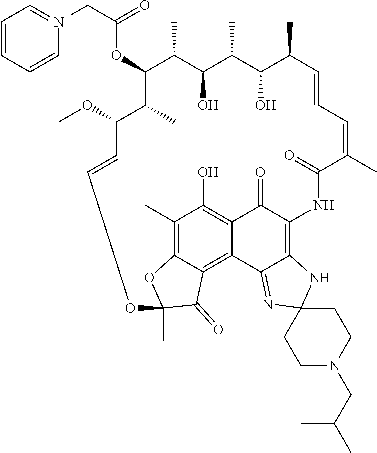 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccccc2)[C@@H]1C)C4=O] |
| 38 | 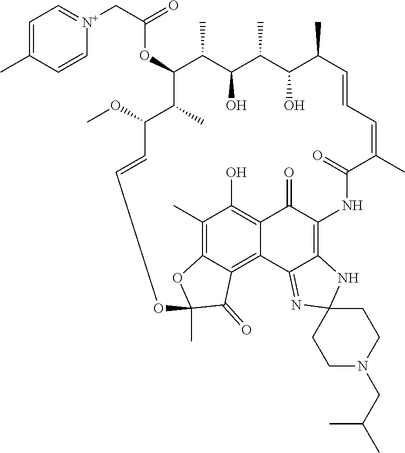 |
| 39 | 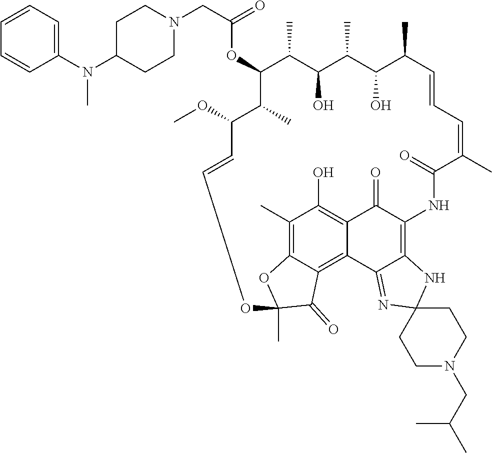 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)c3ccccc3)CC2)[C@@H]1C)C4=O] |
| 40 | 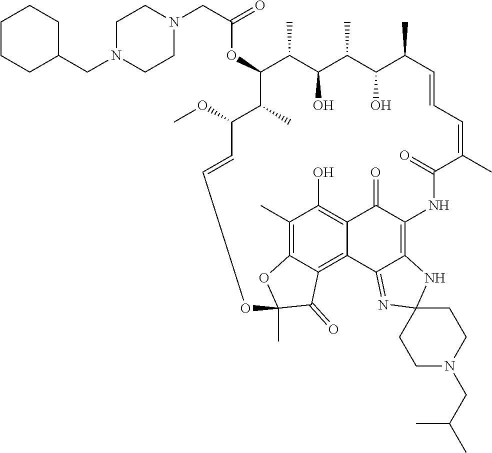 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(CC3CCCCC3)CC2)[C@@H]1C)C4=O] |

In some preferred embodiments of any of the above aspects, the compound of Formula I is selected from the group consisting of:

| Cpd | Structure |
|---|---|
| 3 | 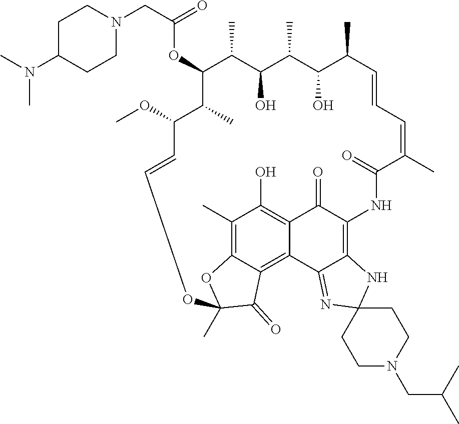 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)C)CC2)[C@@H]1C)C4=O] |
| 6 | 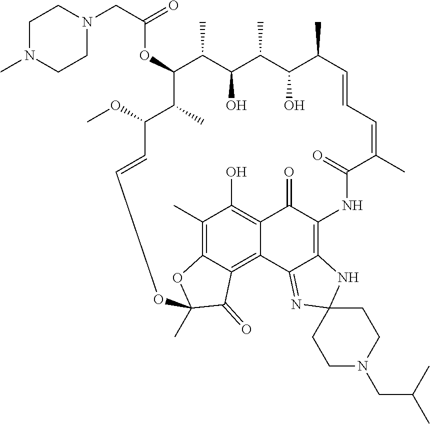 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(C)CC2)[C@@H]1C)C4=O] |
| 7 | 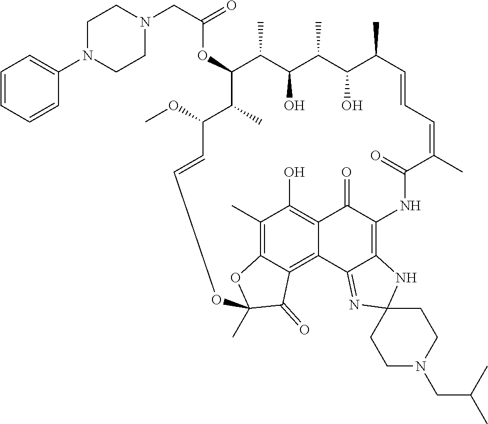 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(c3ccccc3)CC2)[C@@H]1C)C4=O] |
| 8 | 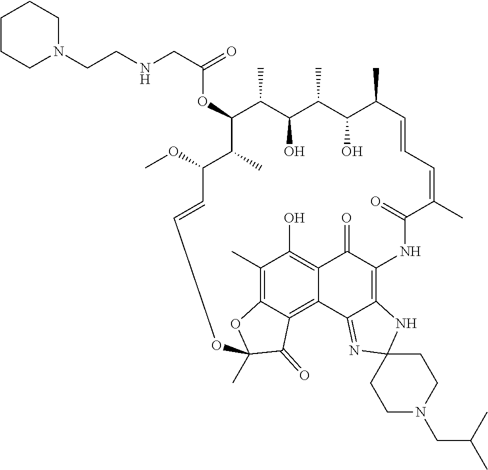 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCCCC2)[C@@H]1C)C4=O] |
| 10 | 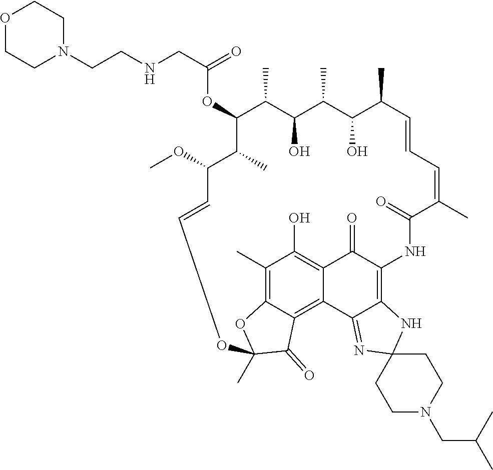 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCOCC2)[C@@H]1C)C4=O] |
| 12 | 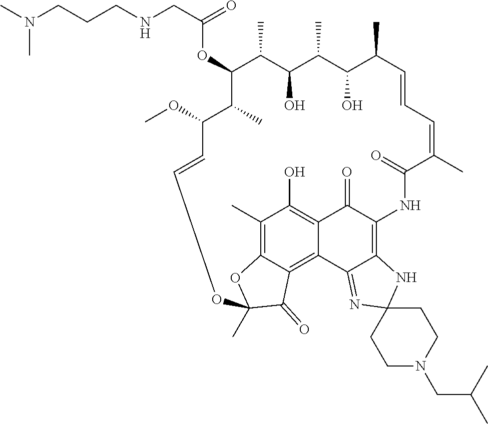 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN(C)C)[C@@H]1C)C4=O] |
| 18 | 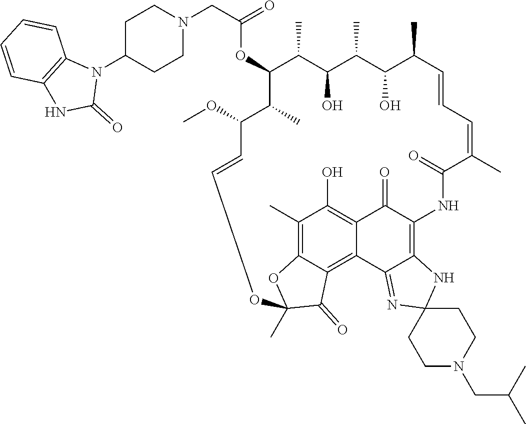 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5ccccc53)CC2)[C@@H]1C)C4=O] |
| 19 | 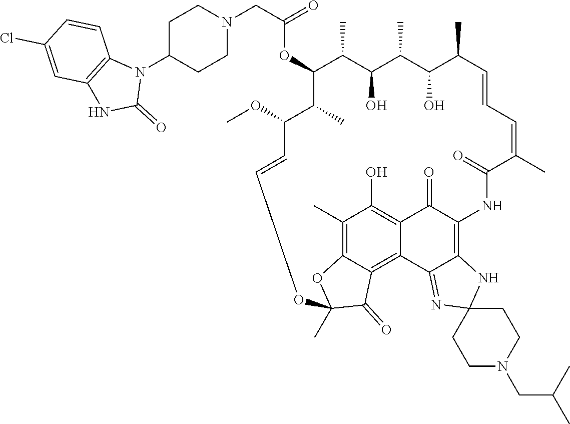 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5cc(Cl)ccc53)CC2)[C@@H]1C)C4=O] |
| 20 | 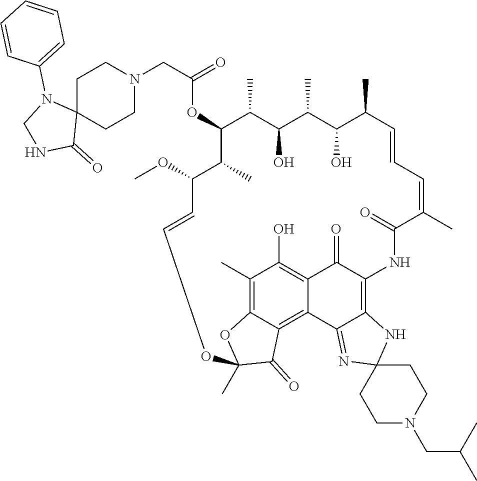 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3(CC2)C(=O)NCN3c2ccccc2)[C@@H]1C)C4=O] |
| 23 | 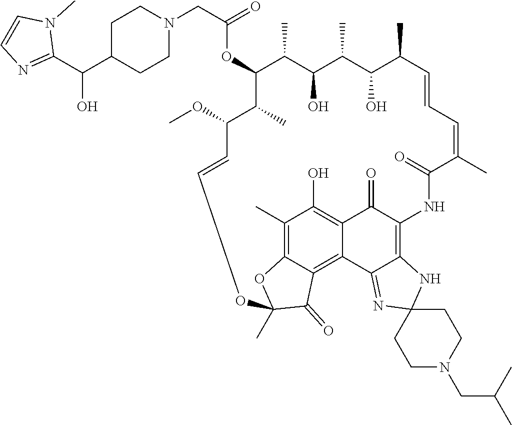 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(C(O)c3nccn3C)CC2)[C@@H]1C)C4=O] |
| 24 | 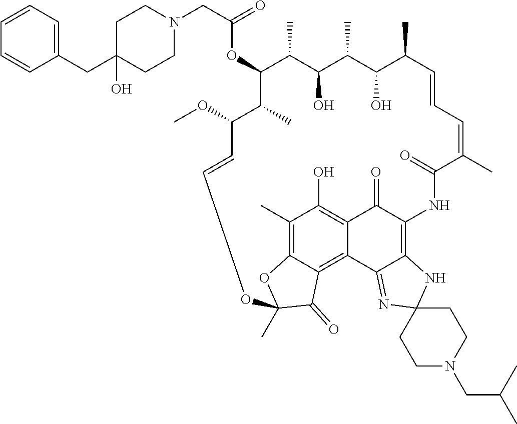 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(O)(Cc3ccccc3)CC2)[C@@H]1C)C4=O] |
| 25 | 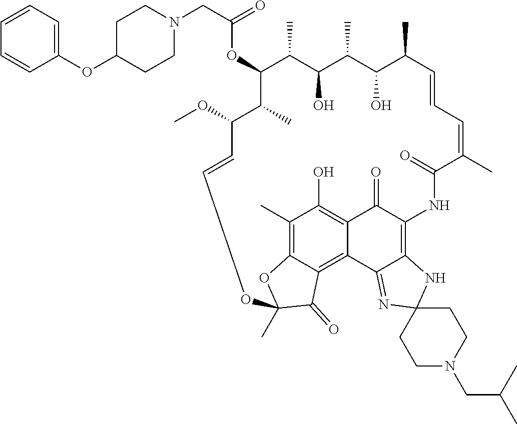 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Oc3ccccc3)CC2)[C@@H]1C)C4=O] |
| 26 | 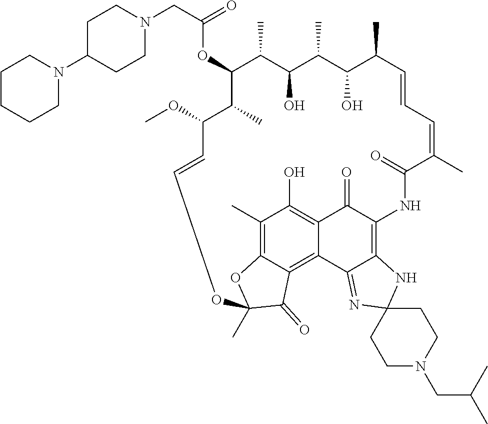 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCCCC3)CC2)[C@@H]1C)C4=O] |
| 27 | 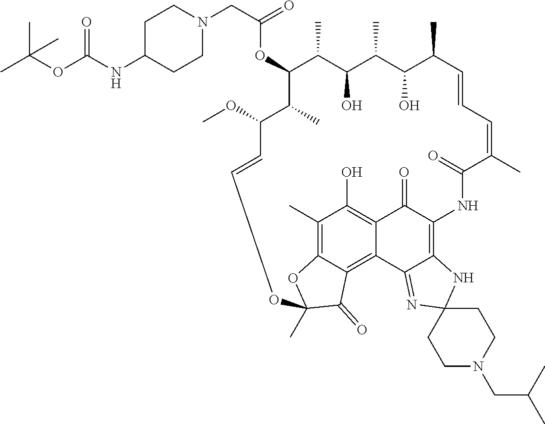 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NC(=O)OC(C)(C)C)CC2)[C@@H]1C)C4=O] |
| 28 | 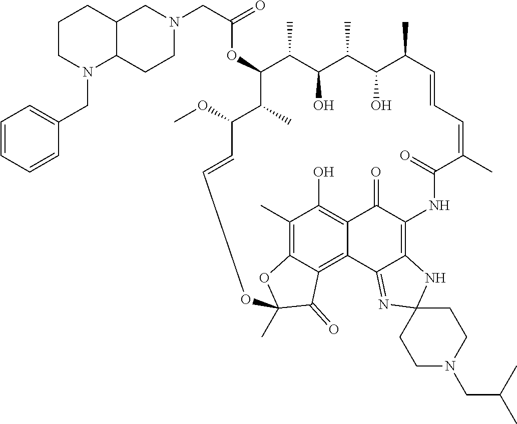 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3C(CCCN3Cc3ccccc3)C2)[C@@H]1C)C4=O] |
| 29 | 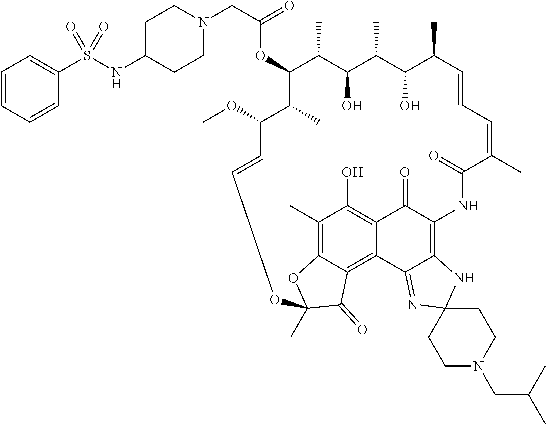 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NS(=O)(=O)c3ccccc3)CC2)[C@@H]1C)C4=O] |
| 30 | 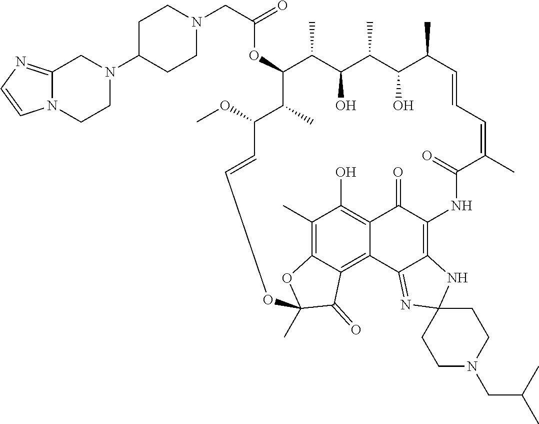 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCn5ccnc5C3)CC2)[C@@H]1C)C4=O] |
| 33 | 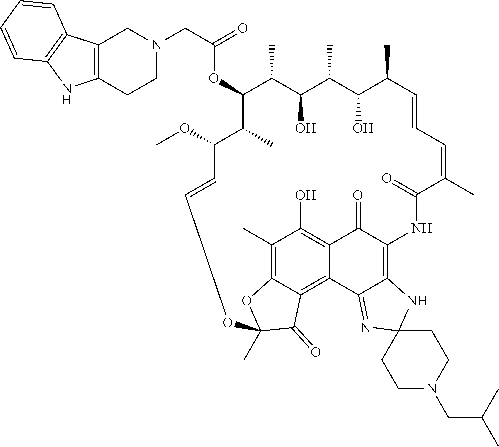 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCc3[nH]c5ccccc5c3C2)[C@@H]1C)C4=O] |
| 34 | 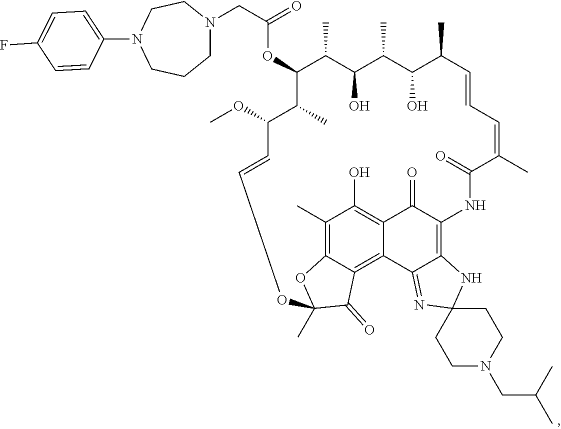 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCN(c3ccc(F)cc3)CC2)[C@@H]1C)C4=O] |
| 35 | 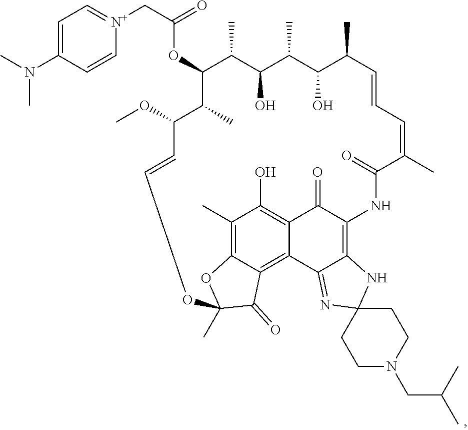 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccc(N(C)C)cc2)[C@@H]1C)C4=O] |
| 39 | 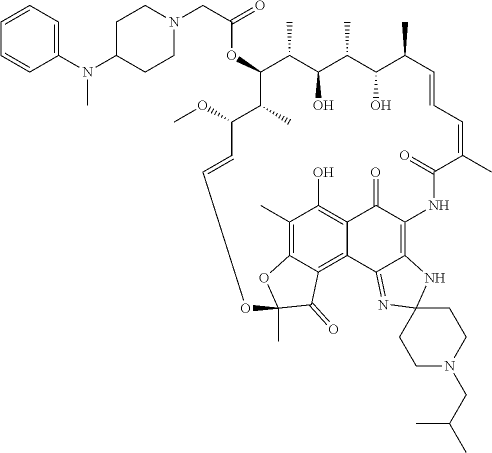 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)c3ccccc3)CC2)[C@@H]1C)C4=O] |
| | and |
| 40 | 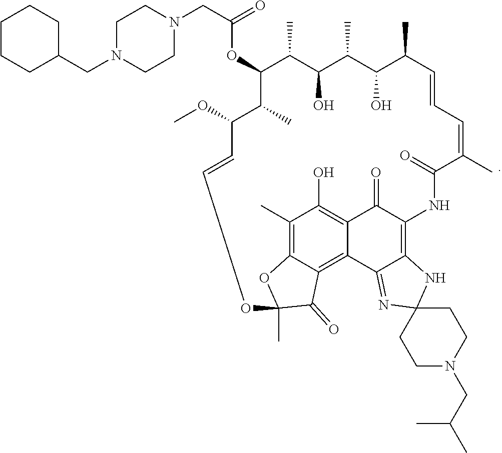 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(CC3CCCCC3)CC2)[C@@H]1C)C4=O] |

In one aspect, the present invention provides a pharmaceutical composition comprising at least one compound of Formula I according to the present invention, or a pharmaceutically acceptable salt, tautomer, solvate or hydrate thereof, and a pharmaceutically acceptable excipient.

In one aspect, the present invention provides a compound according to Formula I of the present invention or a pharmaceutically acceptable salt, tautomer, solvate or hydrate thereof, or a pharmaceutical composition comprising a compound of the present invention, for use in the prevention or treatment of a disease in a subject, preferably an infection, further preferably a bacterial infection, yet further preferably a bacterial infection caused by one or more bacterium belonging to a genus of non-tuberculous Mycobacteria, preferably M. abscessus.

In one aspect, the present invention provides a method of preventing or treating a bacterial infection in a subject in need thereof, comprising administering to the subject an effective amount of a compound of Formula I of the present invention or a pharmaceutically acceptable salt, tautomer, solvate, or hydrate thereof. In one aspect, the present invention provides a method of treating a bacterial infection in a subject in need thereof, comprising administering to the subject an effective amount of a pharmaceutical composition comprising a compound of Formula I of the present invention or a pharmaceutically acceptable salt, tautomer, solvate, or hydrate thereof.

In one aspect, the present invention provides the use of a compound of Formula I of the present invention or a pharmaceutically acceptable salt, tautomer, solvate, or hydrate thereof in the manufacture of a medicament for preventing or treating a bacterial infection in a subject in need thereof. In one aspect, the present invention provides the use of a pharmaceutical composition comprising a compound of the present invention or a pharmaceutically acceptable salt, tautomer, solvate, or hydrate thereof in the manufacture of a medicament for treating a bacterial infection in a subject in need thereof.

In some embodiments, the bacterial infection is caused by one or more bacterium belonging to a genus of non-tuberculous Mycobacteria, preferably M. abscessus. In some embodiments, the bacterial infection is caused by A. baumannii.

### Methods of Synthesizing the Disclosed Compounds

The compounds of the present invention may be made by a variety of methods, including standard chemistry. The methods include but are not limited to the methods described in the suitable synthetic routes depicted in the schemes given below.

The compounds of the present invention may be prepared by methods known in the art of organic synthesis as set forth in part by the following synthetic schemes and examples. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of the present invention.

Those skilled in the art will recognize if a stereocenter exists in the compounds of Formula I. Accordingly, the present invention includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley—Interscience, 1994).

The compounds described herein may be made from commercially available starting materials or synthesized using known organic, inorganic, and/or enzymatic processes.

### Preparation of Compounds

Compounds of the present invention can be synthesized by following the steps outlined in General Schemes 1, 2, 3 and 4. Starting materials are either commercially available or made by known procedures in the reported literature or as illustrated.

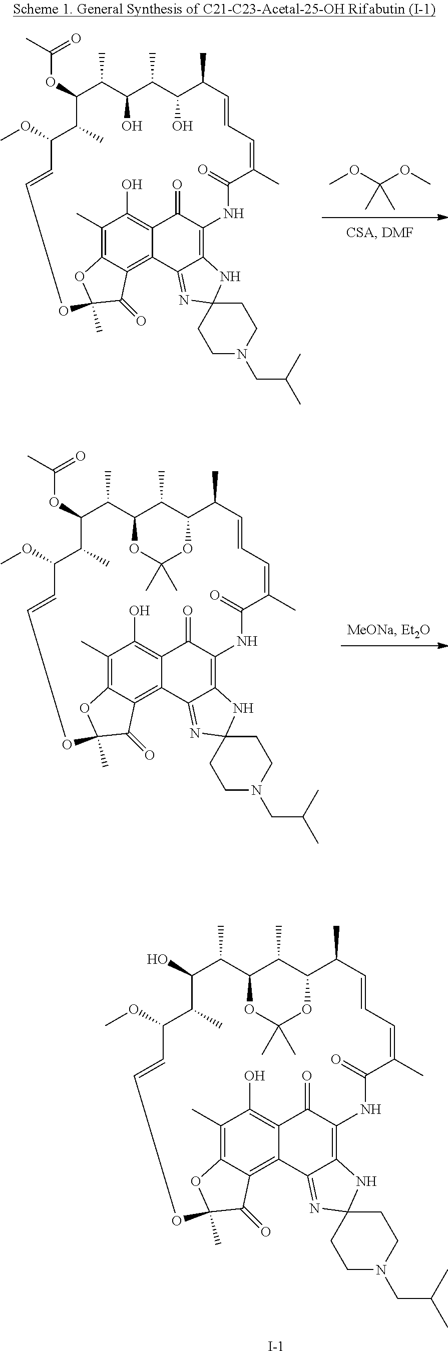

A general method of preparing C21-C23-Acetal-25-OH rifabutin is shown above in Scheme 1. Protection of the C21 and C23 hydroxy groups using dimethoxy propane and camphorsulphonic acid in DMF afforded an acetal-protected rifabutin. De-acetylation of the protected rifabutin using sodium methoxide in ether afforded the 25-OH rifabutin with the C21 and C23 hydroxy groups protected (I-1).

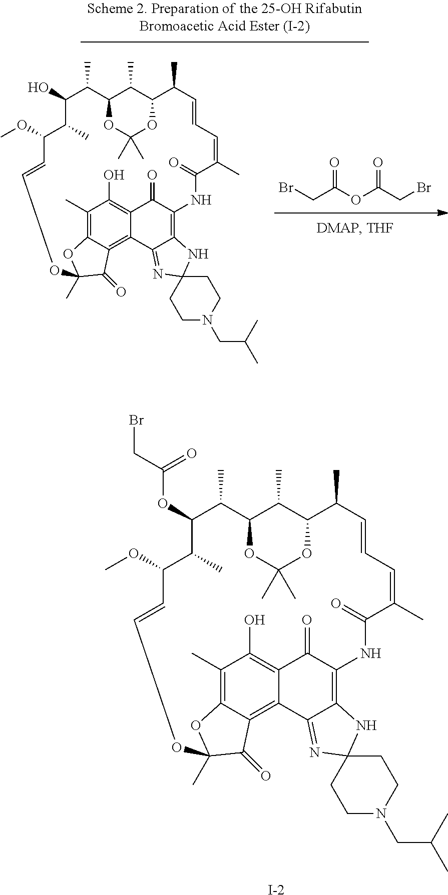

C21-C23-protected, 25-OH rifabutin (I-1) can be esterified using an appropriate carboxylic acid anhydride such as bromoacetic anhydride in the presence of a suitable base such as an amine base, e.g., 4-(dimethylamino)pyridine.

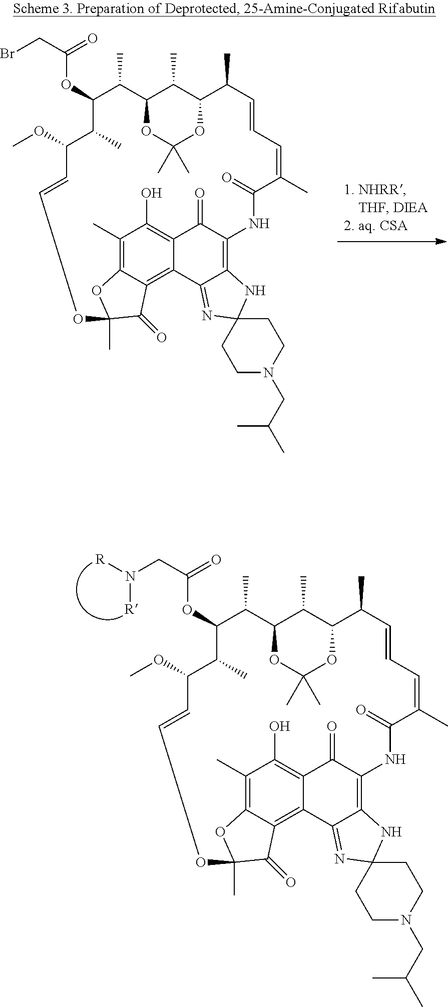

The C25-esterified, protected rifabutin can be condensed with an appropriate amine in presence of DIEA in THF. Deprotection of the C21-C23 acetal in the conjugated rifabutin can be carried out by treating with an acid such as camphorsulphonic acid in water.

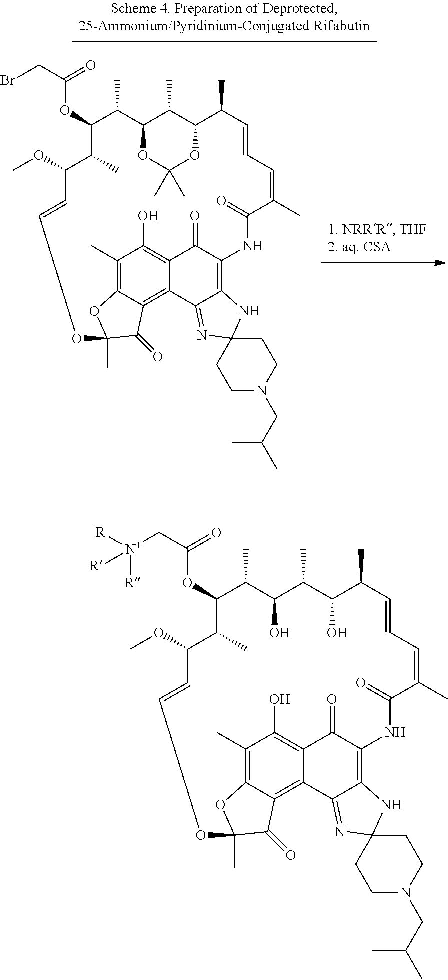

The C25-esterified, protected rifabutin can be condensed with an appropriate tertiary amine or pyridine in THF. Deprotection of the C21-C23 acetal in the conjugated rifabutin can be carried out by treating with an acid such as camphorsulphonic acid in water.

### Antibacterial Efficacy of the Disclosed Compounds

The inventive compounds are C25-modified analogs of rifabutin that exhibit broad spectrum antibacterial activity characteristic of the rifamycin class. Additionally, the inventive compounds unexpectedly showed enhanced antibacterial activity against non-tuberculous Mycobacteria including M. abscessus compared to currently available antibiotics (e.g., rifabutin).

As shown below in Example 5, Table 6, the compounds of the invention are effective at inhibiting bacterial growth in strains of S. aureus, M. abscessus, A. baumannii, M. tuberculosis, M. avium, M. kansasii, M. smegmatis and M. xenopi.

Rifampicin exhibited MIC value above 32 mg/L on M. abscessus and was not considered active against M. abscessus strains. Rifabutin, which is not modified on its C25 position, exhibited modest activity against the tested M. abscessus strains with MIC value of 8 mg/L. In contrast, Compounds of the invention showed MIC values from 0.125 to 4 mg/L, corresponding to 2 to 64-fold increased activity over rifabutin. Accordingly, the present invention teaches compounds that display increased activity against M. abscessus beyond that of antibiotics known in the literature. In some embodiments, the inventive compounds can be used to treat M. abscessus infection, preferably M. abscessus infection that is resistant to current antibiotics.

As shown also in Example 5, Table 6, compounds of the invention retained strong antibacterial activity against A. baumannii, S. aureus, M tuberculosis and other non-tuberculous mycobacterial species such as M. smegmatis, M. Kansasii, M avium, and M. xenopi.

Accordingly, in some embodiments, the inventive compounds can be used to inhibit bacterial infection, preferably infection caused by a non-tuberculous Mycobacteria, more preferably M. abscessus. In some embodiments, the inventive compounds can be used to inhibit bacterial infection caused by A. baumannii.

### Methods of Using the Disclosed Compounds

An aspect of the present invention relates to a compound of Formula I or a pharmaceutically acceptable salt thereof for use as a medicament. An aspect of the present invention relates to methods of treating a bacterial infection in a patient in need thereof. The method involves administering to a patient a compound of Formula I the present invention. The present invention also relates to a compound or a pharmaceutical composition of Formula I as described herein for use in a method for treating a bacterial infection, or for use in the manufacture of a medicament for treating a bacterial infection.

In some embodiments, the infection is caused by one or more bacterium belonging to the genera Mycobacterium spp., Acinetobacter spp., Clostridium spp., Enterococcus spp., Hemophilus spp., Legionella spp., Neisseria spp., Staphylococcus spp., Streptococcus spp., Listeria monocytogenes, Moraxella catarrhalis, Bacillus spp., Bacteroides spp., Gardnerella vaginalis, Lactobacillus spp., Mobiluncus spp., Helicobacter pylori, Campylobacter jejuni, Chlamydia trachomatis and/or Toxoplasma gondii.

In some preferred embodiments, the bacterial infection is caused by one or more bacterium belonging to the genus Acinetobacter, Staphylococcus, and/or Mycobacteria. In some preferred embodiments, the bacterial infection is caused by one or more bacterium belonging to the species A. baumannii, and/or S. aureus, and/or a genus of non-tuberculous Mycobacteria, preferably M. abscessus. In preferred embodiments, the bacterial infection is caused by one or more bacterium belonging to a genus of non-tuberculous Mycobacteria, preferably M. abscessus. In some embodiments, the infection is caused by one or more bacterium belonging to the genus Acinetobacter and/or Staphylococcus, preferably A. baumannii and/or S. aureus.

In some preferred embodiments, the infection is caused by one or more bacterium belonging to a genus of non-tuberculosis Mycobacterium, preferably M. abscessus, M avium, M. kansasii, M. smegmatis, M. xenopi and/or M. malmoense, yet more preferably M. abscessus.

In some embodiments, the infection is caused by one or more bacterium belonging to the species M. abscessus, A. baumannii, and/or S. aureus. In some embodiments, the infection is caused by one or more bacterium belonging to the species M. abscessus. In some embodiments, the infection is caused by one or more bacterium belonging to the species A. baumannii.

In some preferred embodiments, the inventive compounds are used for the treatment of a non-tuberculous Mycobacteria that causes a non-tuberculous Mycobacteria pulmonary infection. In some embodiments the non-tuberculosis Mycobacterium is M. abscessus, M. avium, M. kansasii, M. smegmatis, M. xenopi and/or M. malmoense. Accordingly, in some preferred embodiments, the inventive compounds are for use in the treatment of a non-tuberculous Mycobacteria pulmonary infection. In some preferred embodiments, the inventive compounds are for use in the manufacture of a medicament for the treatment of a non-tuberculous Mycobacteria pulmonary infection. In some preferred embodiments, the present invention provides a method of treating a non-tuberculous Mycobacteria pulmonary infection in a subject in need thereof, comprising administering to the subject an effective amount of an inventive compound of the invention.

Illustrative pharmaceutical compositions are tablets and gelatin capsules comprising a compound of the invention and a pharmaceutically acceptable carrier, such as a) a diluent, e.g., purified water, triglyceride oils, such as hydrogenated or partially hydrogenated vegetable oil, or mixtures thereof, corn oil, olive oil, sunflower oil, safflower oil, fish oils, such as EPA or DHA, or their esters or triglycerides or mixtures thereof, omega-3 fatty acids or derivatives thereof, lactose, dextrose, sucrose, mannitol, sorbitol, cellulose, sodium, saccharin, glucose and/or glycine; b) a lubricant, e.g., silica, talcum, stearic acid, its magnesium or calcium salt, sodium oleate, sodium stearate, magnesium stearate, sodium benzoate, sodium acetate, sodium chloride and/or polyethylene glycol; for tablets also; c) a binder, e.g., magnesium aluminum silicate, starch paste, gelatin, tragacanth, methylcellulose, sodium carboxymethylcellulose, magnesium carbonate, natural sugars such as glucose or beta-lactose, corn sweeteners, natural and synthetic gums such as acacia, tragacanth or sodium alginate, waxes and/or polyvinylpyrrolidone, if desired; d) a disintegrant, e.g., starches, agar, methyl cellulose, bentonite, xanthan gum, algiic acid or its sodium salt, or effervescent mixtures; e) absorbent, colorant, flavorant and sweetener; f) an emulsifier or dispersing agent, such as Tween 80, Labrasol, HPMC, DOSS, caproyl 909, labrafac, labrafil, peceol, transcutol, capmul MCM, capmul PG-12, captex 355, gelucire, vitamin E TGPS or other acceptable emulsifier; and/or g) an agent that enhances absorption of the compound such as cyclodextrin, hydroxypropyl-cyclodextrin, PEG400, PEG200.

The inventive compounds and pharmaceutical compositions may be administered by any suitable route, e.g. orally, for example as a syrup, tablet, capsule, lozenge, controlled-release preparation, fast-dissolving preparation, or lozenge.

Liquid, particularly injectable, compositions can, for example, be prepared by dissolution, dispersion, etc. For example, the disclosed compound is dissolved in or mixed with a pharmaceutically acceptable solvent such as, for example, water, saline, aqueous dextrose, glycerol, ethanol, and the like, to thereby form an injectable isotonic solution or suspension. Proteins such as albumin, chylomicron particles, or serum proteins can be used to solubilize the disclosed compounds.

The disclosed compounds can be also formulated as a suppository that can be prepared from fatty emulsions or suspensions; using polyalkylene glycols such as propylene glycol, as the carrier.

The disclosed compounds can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

Another aspect of the invention relates to a pharmaceutical composition comprising a compound of the present invention and a pharmaceutically acceptable carrier. The pharmaceutically acceptable carrier can further include an excipient, diluent, or surfactant.

Compositions can be prepared according to conventional mixing, granulating or coating methods, respectively, and the present pharmaceutical compositions can contain from about 0.1% to about 99%, from about 5% to about 90%, or from about 1% to about 20% of the disclosed compound by weight or volume.

The dosage regimen utilizing the disclosed compound is selected in accordance with a variety of factors including type, species, age, weight, sex and medical condition of the patient; the severity of the condition to be treated; the route of administration; the renal or hepatic function of the patient; and the particular disclosed compound employed. A physician or veterinarian of ordinary skill in the art can readily determine and prescribe the effective amount of the drug required to prevent, counter or arrest the progress of the condition.

Effective dosage amounts of the disclosed compounds, when used for the indicated effects, range from about 0.5 mg to about 5000 mg of the disclosed compound as needed to treat the condition. Compositions for in vivo or in vitro use can contain about 0.5, 5, 20, 50, 75, 100, 150, 250, 500, 750, 1000, 1250, 2500, 3500, or 5000 mg of the disclosed compound, or, in a range of from one amount to another amount in the list of doses. In one embodiment, the compositions are in the form of a tablet that can be scored.

### EQUIVALENTS

While the present technology has been described in conjunction with the specific embodiments set forth above, many alternatives, modifications and other variations thereof will be apparent to those of ordinary skill in the art. All such alternatives, modifications and variations are intended to fall within the spirit and scope of the present invention.

### EXAMPLES

The invention will now be illustrated by way of the following non-limiting examples. While particular embodiments of the invention are described below, a skilled person will appreciate that various changes and modifications can be made. References to preparations carried out in a similar manner to, or by the general method of, other preparations, may encompass variations in routine parameters such as time, temperature, workup conditions, minor changes in reagents amounts, and the like.

### Abbreviations

The following list provides definitions of certain abbreviations and symbols as used herein. It will be appreciated that the list is not exhaustive, but the meaning of those abbreviations and symbols not herein below defined will be readily apparent to those skilled in the art. In describing the invention, chemical elements are identified in accordance with the Periodic Table of the Elements.
ACN acetonitrile
AcOH acetic acid
aq. aqueous
Ar argon
COMU 1-cyano-2-ethoxy-2-oxoethylidenaminooxy)dimethylamino-morpholino-carbenium hexafluorophosphate
CSA camphorsulphonic acid
DCC dicyclohexylcarbodiimide
DCM dichloromethane
DIEA N,N-diisopropylethylamine
DMF dimethylformamide
EDCI 1-ethyl-3-(3-dimethylaminopropyl)carbodiimide
eq. equivalent
EtOAc ethyl acetate
EtOH ethanol
Et₂O diethylether
h hour(s)
HPLC high performance liquid chromatography
i-PrOH isopropanol
LC liquid chromatography
M molar
MS mass spectroscopy
min minutes
NMR nuclear magnetic resonance spectroscopy
rt room temperature
Rt retention time
sat. saturated

Unless specified otherwise, the purity and identity of Intermediate or example compounds were assessed by state-of-the-art HLPC-MS. Methods are described below.

### Characterization of the Compounds

### HPLC Analysis

Reaction monitoring and compound purity determination were performed by liquid chromatography/mass spectrometry (LC/MS), as follows:

HPLC system: Waters 2695 LC with photodiode array detector Waters 996; Column: XBridge C18 column (3.5 μm particle size, dimensions 50 mm×4.6 mm); Mobile phases: phase A (H₂O/ammonium formate, pH 3.75 (A) or 9.2 (B)) and phase B (CH₃CN+5% H₂O/ammonium formate, pH 3.75 (A) or 9.2 (B)) were used according to the following methods:

| Method A | | | Flow | Method B | | | Flow |
|---|---|---|---|---|---|---|---|
| Time | Phase | Phase | (mL/ | Time | Phase | Phase | (mL/ |
| (min) | A | B | min) | (min) | A | B | min) |
| 0.00 | 100.0 | 0.0 | 2.00 | 0.00 | 100.0 | 0.0 | 2.00 |
| 0.25 | 100.0 | 0.0 | 2.00 | 1.00 | 100.0 | 0.0 | 2.00 |
| 3.50 | 0.0 | 100.0 | 2.00 | 25.00 | 0.0 | 100.0 | 2.00 |
| 4.00 | 0.0 | 100.0 | 2.00 | 27.00 | 0.0 | 100.0 | 2.00 |
| 4.05 | 100.0 | 0.0 | 2.00 | 27.10 | 100.0 | 0.0 | 2.00 |
| 5.00 | 100.0 | 0.0 | 2.00 | 30.00 | 100.0 | 0.0 | 2.00 |

Method A was used for the intermediates. Method B was used to measure final compounds purity.

### Mass Spectrometry Analysis

Mass spectrometer: Waters Alliance Micromass ZQ 2000. Ionization: electrospray (polarity: negative and positive).

Purity (%) was determined by reversed phase HPLC, using UV detection (254 nm). Structure was confirmed by MS, using electro spray ionization positive (ESI+) method and reported as [M+H]⁺, referring to the protonated molecular ion.

### NMR Analysis

NMR spectra were recorded on a Bruker DRX-300 spectrometer. Chemical shifts are in parts per million (ppm). The assignments were made using one-dimensional (1D) ¹H and ¹³C spectra and two-dimensional (2D) HSQC, HMBC spectra.

### Compound Synthesis

### Example 1

### Synthesis of C21-C23 Acetal 25-OH Rifabutin (I-1)

The compounds of the current invention were obtained from the free 25-OH rifabutin (Intermediate I-1) prepared from commercially available rifabutin as shown below.

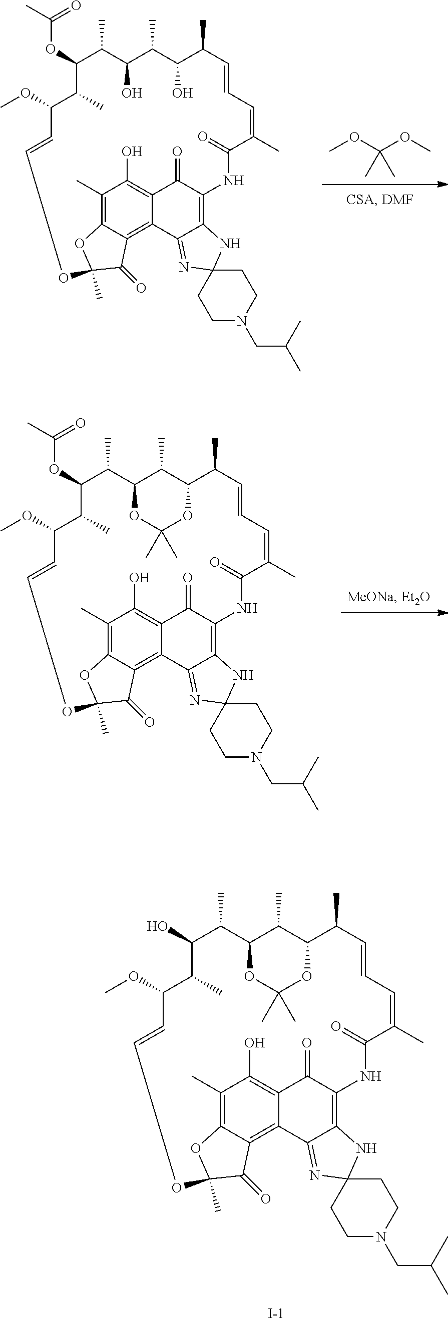

### Synthesis of Intermediate I-1

Rifabutin (40.0 g, 47.2 mmol) was dissolved in dry DMF (80 mL) at rt under nitrogen atmosphere. 2,2-Dimethoxy-propane (58.1 mL, 472 mmol) and camphorsulphonic acid (12.6 g, 54.3 mmol) were sequentially added to the solution. The reaction mixture was stirred at rt for 26 h under nitrogen atmosphere. The mixture was then cooled at 0° C. and poured into a mixture of sat. aq. solution of NaHCO₃ (700 mL) and water (500 mL). The reaction flask was washed with acetone (100 mL). The resulting suspension was stirred on an ice bath for 10 min and filtered. The cake was rinsed with sat. aq. solution of NaHCO₃ (100 mL) and water (50 mL) and dried under vacuum at 40° C. for 24 h. The crude product was purified by flash chromatography (DCM to 50% of the mixture DCM/MeOH/NH₄OH 90/9/1.5 in DCM) to afford the desired product as a purple solid (37.68 g, 90% yield).

LC/MS A (ESI+): tr=3.20 min, m/z [M+H]⁺=887.48, UV purity: 97% (254 nm).

¹H NMR (300 MHz, CDCl₃): δ 0.39 (d, J=7.0 Hz, 3H), 0.68 (d, J=7.0 Hz, 3H), 0.77-0.88 (m, 9H), 0.89-0.98 (m, 6H), 1.23 (s, 3H), 1.36-1.55 (m, 2H), 1.71-2.18 (m, 6H), 1.77 (s, 3H), 1.96 (s, 3H), 2.04 (s, 3H), 2.22-2.35 (m, 3H), 2.31 (s, 3H), 2.52-2.72 (m, 2H), 2.79 (s, 3H), 2.91-3.09 (m, 2H), 3.06 (dd, J=10.3, 5.4 Hz, 1H), 3.34 (d, J=6.7 Hz, 1H), 3.58 (dd, J=3.1 Hz, 1H), 4.91 (d, J=7.5 Hz, 1H), 5.08 (dd, J=12.1, 6.7 Hz, 1H), 5.87 (d, J=12.2 Hz, 1H), 6.08 (dd, J=15.5, 6.8 Hz, 1H), 6.16 (dd, J=10.7, 1.4 Hz, 1H), 6.28 (dd, J=15.5, 10.7 Hz, 1H), 7.75 (s, 1H), 8.75 (s, 1H), 14.82 (s, 1H).

¹³C NMR (75 MHz, CDCl₃): δ 7.89, 9.11, 9.99, 12.8, 18.0, 20.2, 20.3, 20.9, 23.5, 25.8, 34.1, 35.4, 36.0, 36.8, 40.8, 41.4, 51.5, 56.2, 66.3, 71.2, 74.6, 76.9, 78.9, 95.0, 100.1, 104.6, 106.1, 108.7, 111.6, 113.7, 115.3, 123.8, 125.5, 131.2, 132.6, 140.8, 140.9, 142.3, 155.2, 168.2, 168.9, 170.5, 172.2, 181.3, 192.7.

21,23-Dimethylacetonide-rifabutin (10.6 g, 11.9 mmol) was dissolved in dry diethyl ether (500 mL). The solution was cooled to −10° C. with Ar bubbling. After 15 min, a solution of NaOMe (30 mL, 25 w % in MeOH) was slowly added. Precipitation occurred and another portion of diethyl ether (100 mL) was added to homogenize. NaOMe solution was again added (27.4 mL). The solution was stirred at −5° C. for 10 min before the ice bath was removed. The reaction mixture was stirred at rt for 6 h. Sat. aq. solution of NaHCO₃ (400 mL) was added and the layers were separated. The aqueous layer was extracted with diethyl ether (400 mL) and the combined organic layers were washed with brine (300 mL) and evaporated to afford the desired crude product as a black-purple powder. The product was purified by flash chromatography (DCM to 50% of the mixture DCM/MeOH/NH₄OH 90/9/1.5 in DCM). After evaporation, the product was redissolved in acetone and slowly added into water under vigorous stirring. The solid was collected by filtration and dried at 40° C. to yield 8.54 g of the de-acetylated analog of rifabutin I-1.

LC/MS A (ESI+): tr=2.98 min. m/z [M+H]⁺=845.59, UV purity: 97% (254 nm).

¹H NMR (300 MHz, CDCl₃): δ 0.48 (d, J=7.0 Hz, 3H), 0.70 (d, J=6.8 Hz, 3H), 0.74-0.84 (m, 9H), 0.86-0.96 (m, 6H), 1.04 (s, 3H), 1.23-1.33 (m, 1H), 1.43-1.66 (m, 2H), 1.72 (s, 3H), 1.75-2.11 (m, 5H), 2.00 (s, 3H), 2.18-2.29 (m, 3H), 2.21 (s, 3H), 2.47-2.71 (m, 2H), 2.85-3.09 (m, 2H), 3.05-3.14 (m, 1H), 3.10 (s, 3H), 3.27-3.35 (m, 1H), 3.38-3.51 (m, 2H), 3.55 (dd, J=9.2, 3.3 Hz, 1H), 4.93 (dd, J=12.5, 9.5 Hz, 1H), 5.93 (dd, J=15.5, 6.5 Hz, 1H), 6.05-6.16 (m, 2H), 6.24 (dd, J=15.5, 11.7 Hz, 1H), 7.72 (s, 1H), 8.63 (s, 1H), 14.83 (s, 1H).

¹³C NMR (75 MHz, CDCl₃): δ 7.7, 8.4, 12.9, 17.8, 19.9, 20.0, 20.9, 21.0, 24.2, 25.5, 25.8, 34.6, 35.2, 36.3, 39.7, 41.1, 51.4, 51.7, 56.2, 66.3, 71.0, 71.5, 75.5, 83.0, 95.0, 99.6, 104.9, 105.7, 108.6, 111.7, 111.8, 114.3, 124.1, 125.5, 132.0, 132.4, 140.3, 142.6, 142.7, 155.2, 168.2, 169.1, 171.3, 181.6, 191.2.

### Example 2

### Synthesis of 25-Bromo Acetic Ester Rifabutin (I-2)

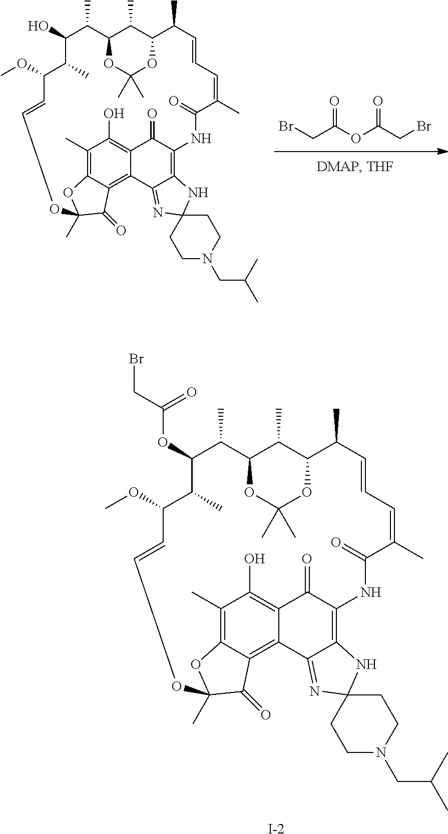

Intermediate I-2 (800 mg, 0.94 mmol, 1 eq) was dissolved in DCM (20 mL) and cold at 0° C. Bromoacetic anhydride (1.54 g, 5.92 mmol, 5 eq) and DMAP (723 mg, 5.92 mmol, 5 eq) were then added and reaction was stirred at 0° C. After 1 h, the mixture was washed with an aqueous solution of HCl-1N (20 mL), a saturated aqueous solution of NaHCO₃ (20 mL) and brine (20 mL). Organic layer was dried over MgSO₄ and concentrated under vacuum. The obtained solid was purified by flash chromatography (CHCl₃/MeOH, from 100/0 to 95/5). Pure fractions were gathered, and solvent evaporated to yield I-2 as a black solid (m=620 mg, yield=54%).

LC/MS A (ESI+): tr=3.48 min, m/z [M+H]⁺=965.48/967.47, UV purity: 95% (254 nm).

¹H NMR (300 MHz, CD₂Cl₂): δ 0.39 (d, J=7.0 Hz, 3H), 0.73 (d, J=7.1 Hz, 3H), 0.79-0.88 (m, 9H), 0.92-0.98 (m, 6H), 1.17 (s, 3H), 1.47-1.60 (m 2H), 1.74 (s, 3H), 1.78-2.07 (m, 6H), 2.03 (d, J=0.8 Hz, 3H), 2.20-2.35 (m, 3H), 2.28 (s, 3H), 2.58-2.76 (m, 2H), 2.83 (s, 3H), 2.88-3.05 (m, 2H), 3.01 (dd, J=10.3, 5.2 Hz, 1H), 3.34-3.41 (m, 1H), 3.59 (dd, J=10.6, 3.2 Hz, 1H), 3.74 (d, J=12.3 Hz, 1H), 3.79 (d, J=12.3 Hz, 1H), 4.96-5.01 (m, 1H), 5.03 (dd, J=12.1, 6.9 Hz, 1H), 5.93 (dd, J=12.1, 0.9 Hz, 1H), 6.05 (dd, J=15.6, 6.9 Hz, 1H), 6.16 (dd, J=10.7, 1.4 Hz, 1H), 6.29 (dd, J=15.6, 10.7 Hz, 1H), 7.75 (s, 1H), 8.77 (s, 1H), 14.87 (s, 1H).

¹³C NMR (75 MHz, CD₂Cl₂): δ 7.8, 9.7, 9.9, 12.9, 17.8, 20.1, 20.3, 20.9, 21.0, 24.0, 25.9, 26.1, 26.7, 34.7, 35.6, 36.3, 36.4, 40.8, 41.0, 51.7, 51.8, 56.3, 66.6, 71.1, 76.6, 77.0, 79.2, 95.1, 100.1, 104.9, 106.1, 108.9, 112.0, 114.0, 114.5, 124.1, 125.8, 131.8, 132.5, 140.9, 141.5, 142.6, 155.5, 166.7, 168.5, 169.0, 172.3, 181.7, 192.5.

The following Intermediates (shown in Table 3) are obtained using the procedure described in Example 2 for Intermediate I-2 and using the corresponding anhydride in place of bromoacetic anhydride, i.e., 2-bromopropanoic anhydride for intermediate I-3 and 2-bromo-2-methylpropanoic anhydride for intermediate I-4.

TABLE 3
Exemplary Intermediates of the Invention

| Intermediate | Structure |
|---|---|
| I-3 | 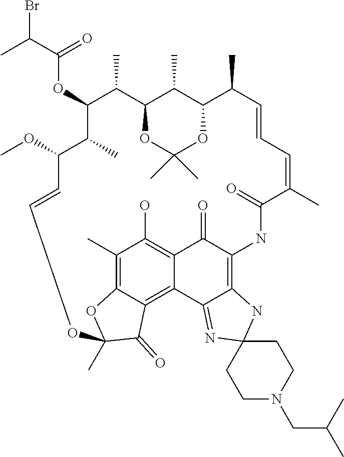 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@@H]2OC(C)(C)O[C@@H]([C@@H](C)[C@H](OC(=O)C(C)Br)[C@@H]1C)[C@@H]2C)C4=O] |
| I-4 | 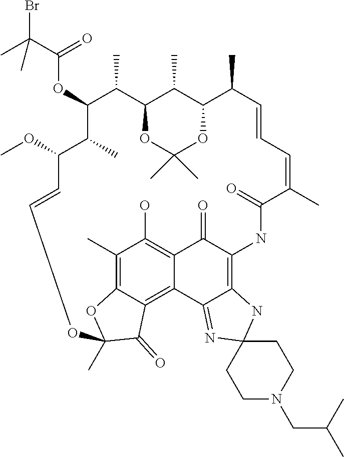 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@@H]2OC(C)(C)O[C@@H]([C@@H](C)[C@H](OC(=O)C(C)(C)Br)[C@@H]1C)[C@@H]2C)C4=O] |

### Example 3

### General Procedure for the Preparation of Amine Derivatives

A solution of Intermediate I-2 in THF (0.035M) was prepared. The corresponding amines (0.020 mmol, 2 eq) were dispensed in 9 mL Kimble glass tubes, and 300 μL of the solution of the rifabutin Intermediate in THF (corresponding to 1 eq of the rifabutin Intermediate) followed by DIEA (3.54 μL, 0.020 mmol, 2 eq) were added. The reaction was stirred at rt. After 12 h, 600 μL of a solution of camphorsulphonic in water (0.5N) was added to the mixtures. The reaction was stirred at rt for 12 h. Each mixture was then deposited on a PoraPak column (Rxn RP 6CC) previously equilibrated with water. The elution was carried out by adding 5 mL of water, 5 mL of a sat. aq. solution of NaHCO₃, 5 mL of a solution of water/ACN (90/10), 5 mL of water and 10 mL of ACN. The 10 mL ACN fractions were collected, frozen and freeze-dried to give the desired products as purple powders.

Table 4 below summarizes exemplary compounds of the invention that were prepared according to the protocols of Example 3 above.

TABLE 4
Compounds (Cpd) of the Invention Obtained from Intermediate I-2, I-3, or I-4.

| Cpd | Structure | MW | Rt | [M + H]+ |
|---|---|---|---|---|
| 1 | 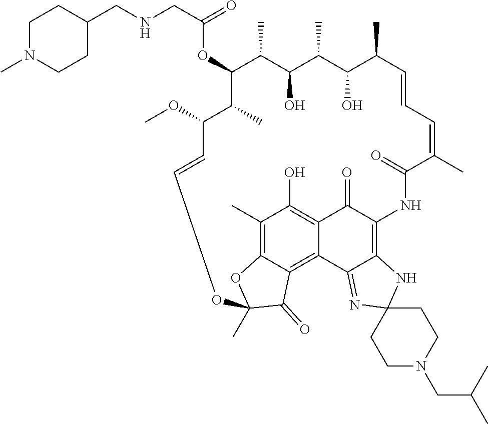 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCC2CCN(C)CC2)[C@@H]1C)C4=O] | 972.52 | 14.02 | 973.82 |
| 2 | 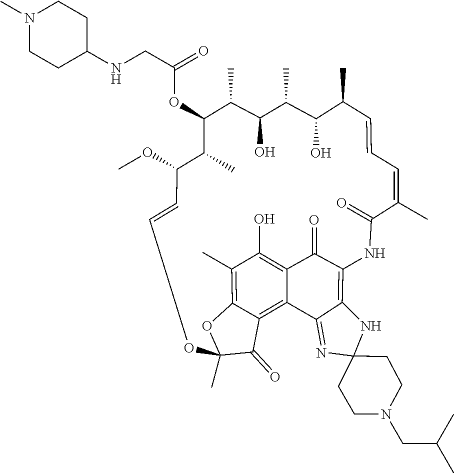 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNC2CCN(C)CC2)[C@@H]1C)C4=O] | 958.54 | 13.97 | 959.80 |
| 3 | 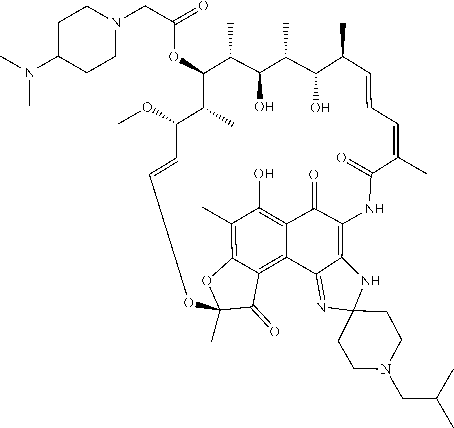 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)C)CC2)[C@@H]1C)C4=O] | 972.55 | 14.62 | 973.82 |
| 4 | 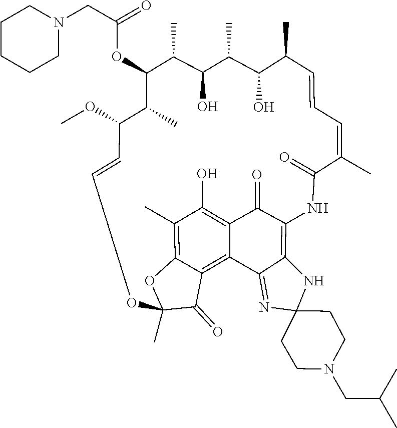 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCCC2)[C@@H]1C)C4=O] | 929.51 | 17.83 | 930.77 |
| 5 | 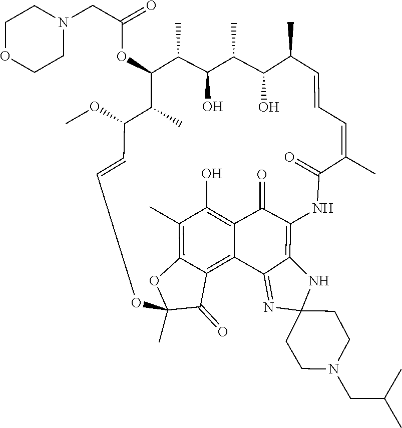 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCOCC2)[C@@H]1C)C4=O] | 931.49 | 15.03 | 932.75 |
| 6 | 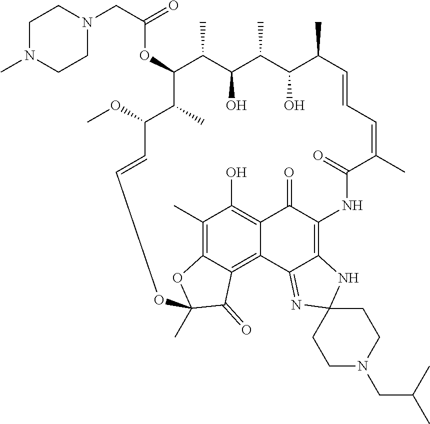 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(C)CC2)[C@@H]1C)C4=O] | 944.52 | 14.07 | 945.79 |
| 7 | 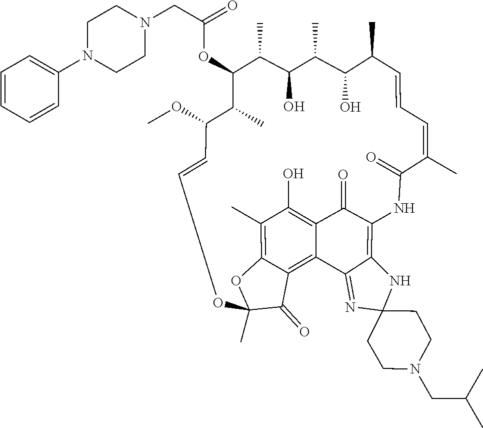 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(c3ccccc3)CC2)[C@@H]1C)C4=O] | 1006.54 | 18.70 | 1007.81 |
| 8 | 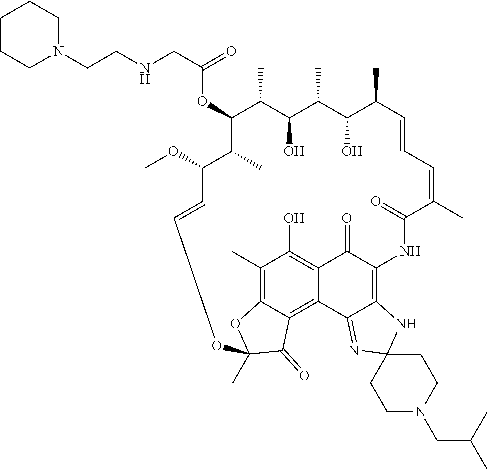 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCCCC2)[C@@H]1C)C4=O] | 972.55 | 15.47 | 973.82 |
| 9 | 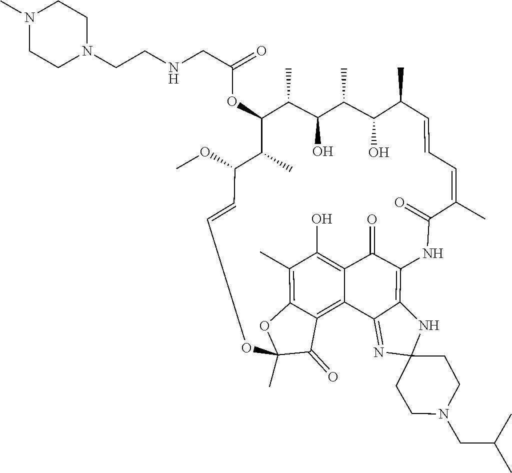 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCN(C)CC2)[C@@H]1C)C4=O] | 987.56 | 13.47 | 988.82 |
| 10 | 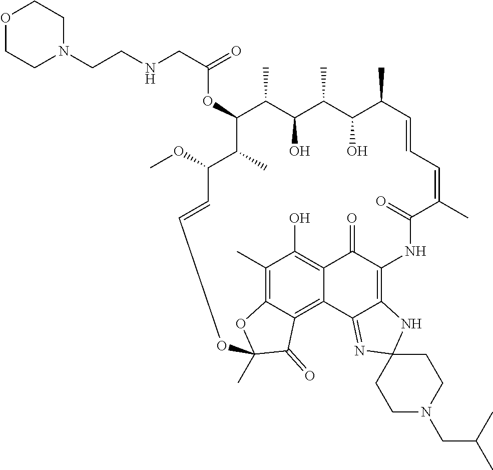 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCOCC2)[C@@H]1C)C4=O] | 974.53 | 14.05 | 975.78 |
| 11 | 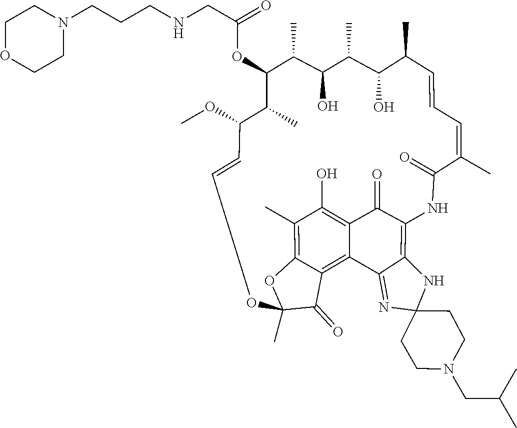 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN2CCOCC2)[C@@H]1C)C4=O] | 988.55 | 14.14 | 989.81 |
| 12 | 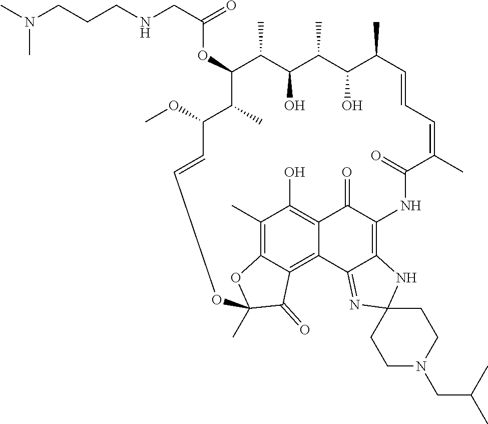 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN(C)C)[C@@H]1C)C4=O] | 946.54 | 14.30 | 947.78 |
| 13 | 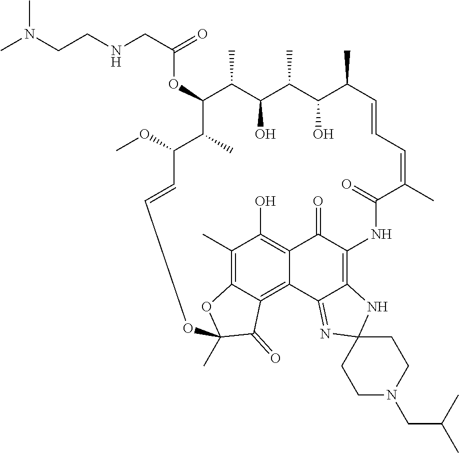 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN(C)C)[C@@H]1C)C4=O] | 932.52 | 14.25 | 933.78 |
| 14 | 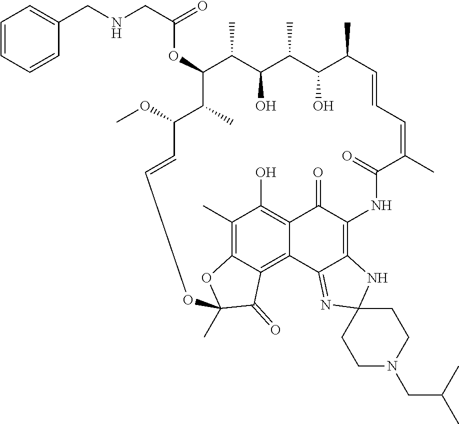 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCc2ccccc2)[C@@H]1C)C4=O] | 951.49 | 17.61 | 952.75 |
| 15 | 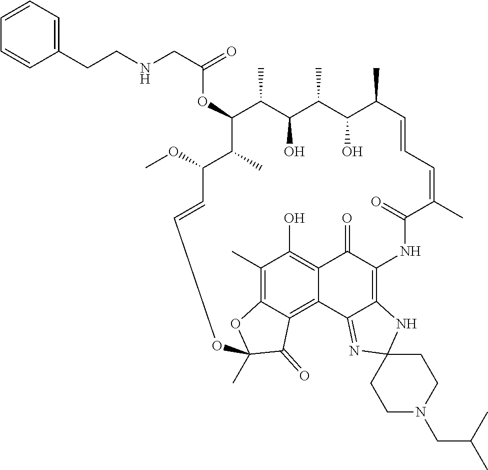 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCc2ccccc2)[C@@H]1C)C4=O] | 965.57 | 17.65 | 966.76 |
| 16 | 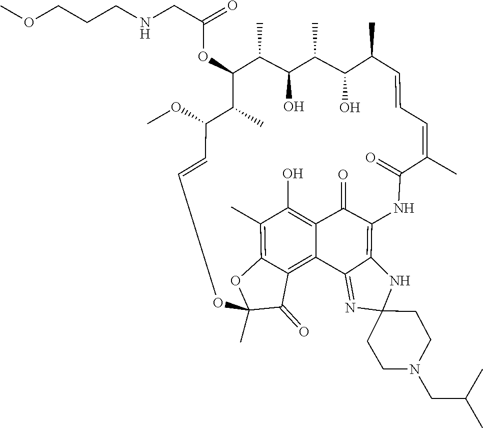 [SMILES: COCCCNCC(=O)O[C@H]1[C@H](C)[C@H](O)[C@H](C)[C@@H](O)[C@@H](C)/C=C/C=C(/C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O] | 933.50 | 15.05 | 934.78 |
| 17 | 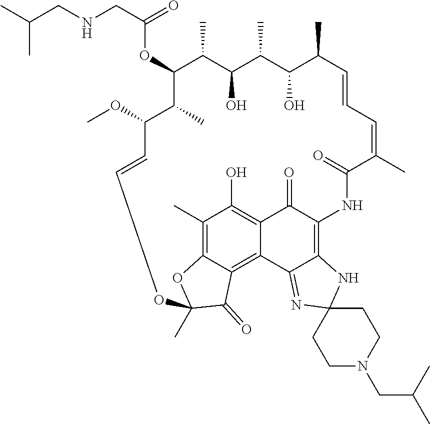 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCC(C)C)[C@@H]1C)C4=O] | 917.51 | 17.25 | 918.76 |
| 18 | 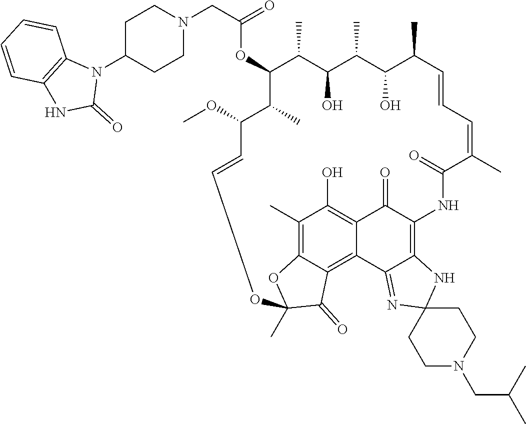 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5ccccc53)CC2)[C@@H]1C)C4=O] | 1061.54 | 16.10 | 1062.79 |
| 19 | 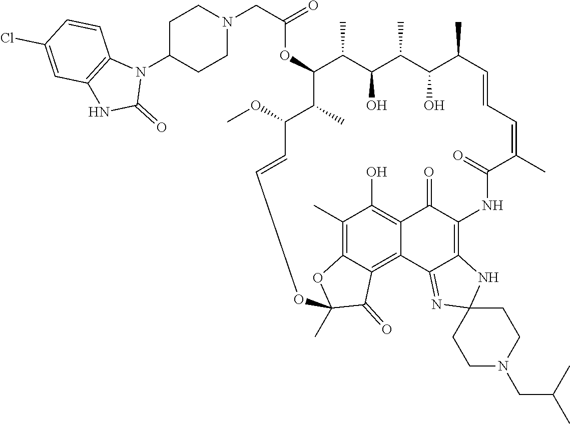 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5cc(Cl)ccc53)CC2)[C@@H]1C)C4=O] | 1095.50 | 17.14 | 1096.76 |
| 20 | 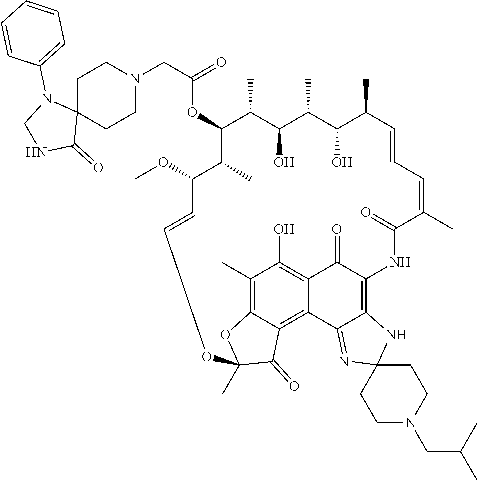 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3(CC2)C(=O)NCN3c2ccccc2)[C@@H]1C)C4=O] | 1075.56 | 16.64 | 1076.60 |
| 21 | 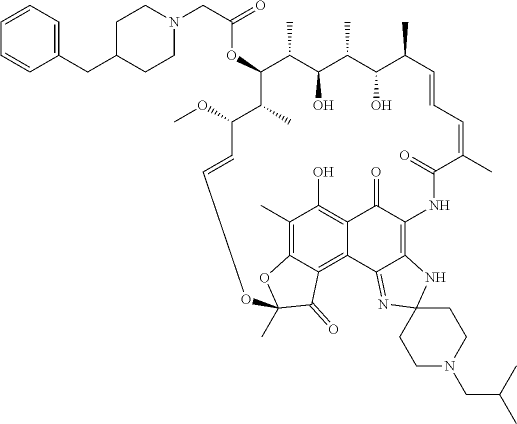 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Cc3ccccc3)CC2)[C@@H]1C)C4=O] | 1019.56 | 20.99 | 1020.81 |
| 22 | 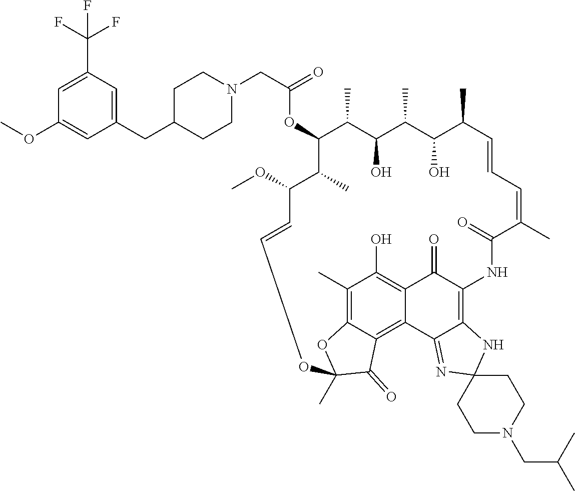 [SMILES: COc1cc(CC2CCN(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)CC2)cc(C(F)(F)F)c1] | 1117.55 | 21.80 | 1118.82 |
| 23 | 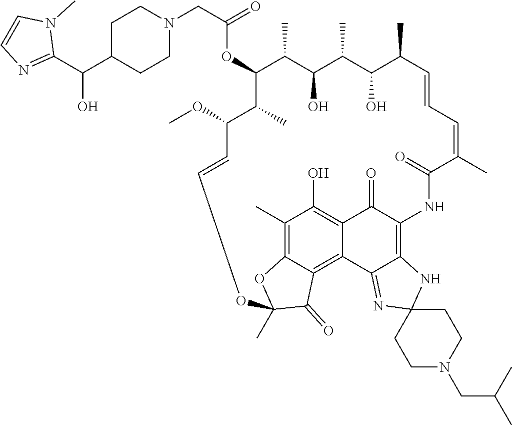 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(C(O)c3nccn3C)CC2)[C@@H]1C)C4=O] | 1039.56 | 14.34 | 1040.80 |
| 24 | 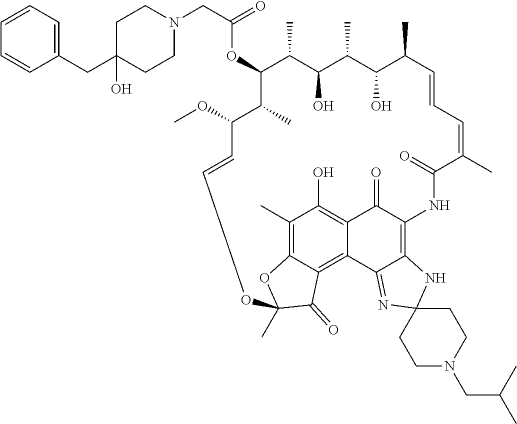 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(O)(Cc3ccccc3)CC2)[C@@H]1C)C4=O] | 1035.55 | 17.07 | 1036.80 |
| 25 | 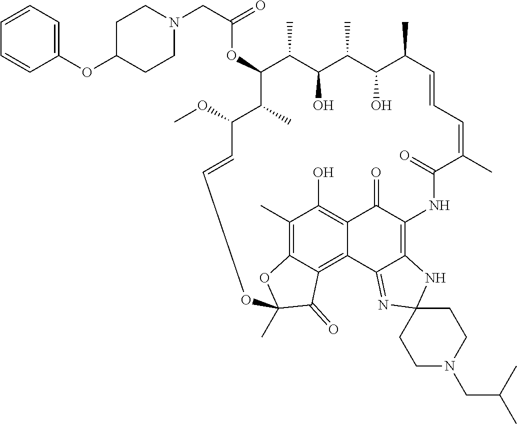 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Oc3ccccc3)CC2)[C@@H]1C)C4=O] | 1021.54 | 19.50 | 1022.78 |
| 26 | 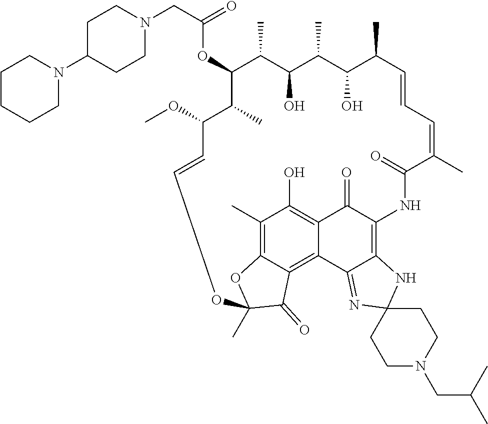 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCCCC3)CC2)[C@@H]1C)C4=O] | 1012.58 | 15.57 | 1013.82 |
| 27 | 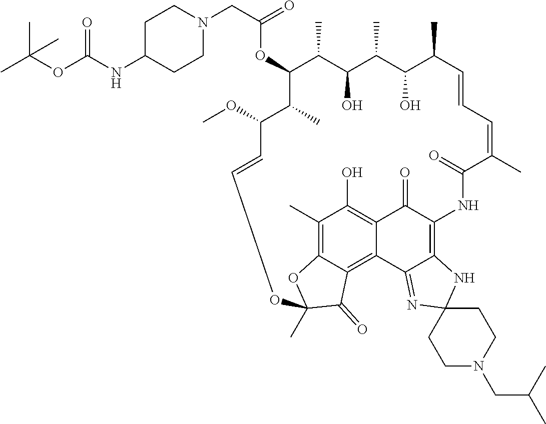 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NC(=O)OC(C)(C)C)CC2)[C@@H]1C)C4=O] | 1044.57 | 17.67 | 1045.84 |
| 28 | 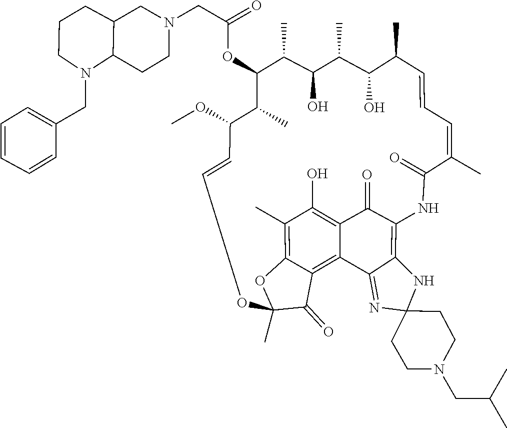 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3C(CCCN3Cc3ccccc3)C2)[C@@H]1C)C4=O] | 1074.60 | 21.17 | 1075.88 |
| 29 | 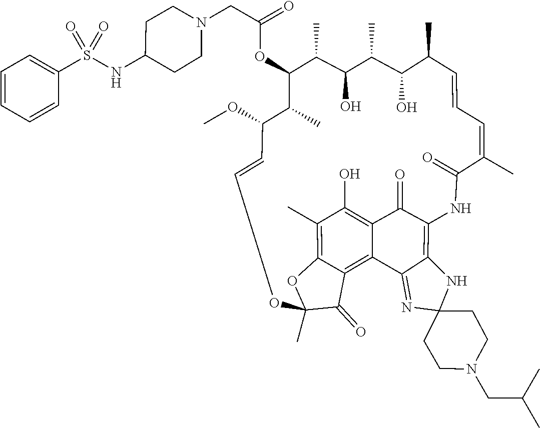 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NS(=O)(=O)c3ccccc3)CC2)[C@@H]1C)C4=O] | 1084.51 | 16.97 | 1085.79 |
| 30 | 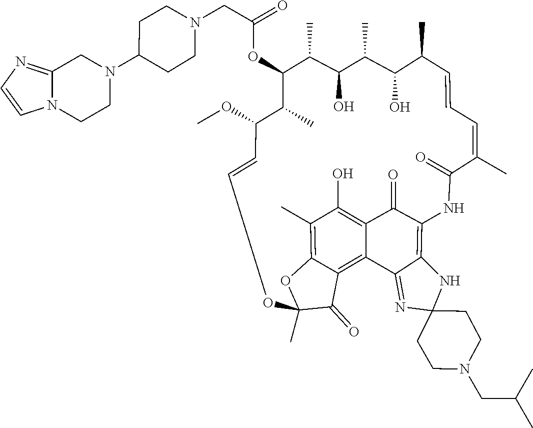 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCn5ccnc5C3)CC2)[C@@H]1C)C4=O] | 1050.57 | 14.27 | 1051.84 |
| 31 | 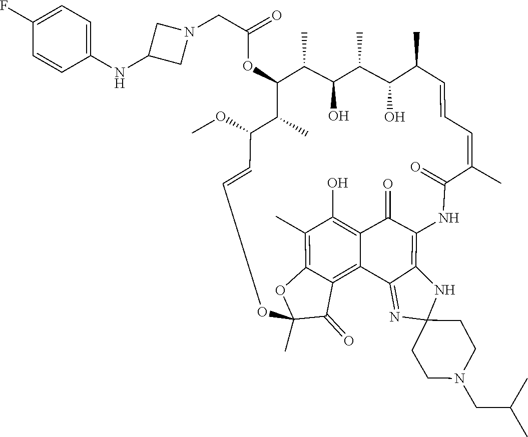 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CC(Nc3ccc(F)cc3)C2)[C@@H]1C)C4=O] | 1010.51 | 17.28 | 1011.77 |
| 32 | 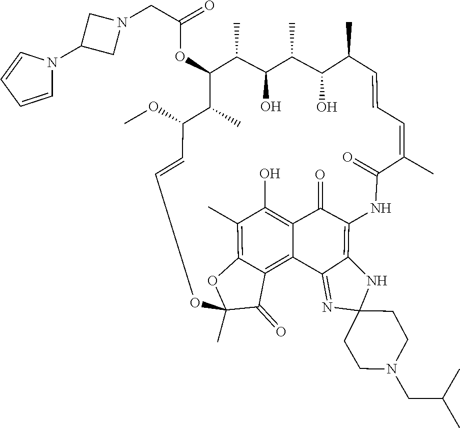 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CC(n3cccc3)C2)[C@@H]1C)C4=O] | 966.51 | 17.44 | 967.76 |
| 33 | 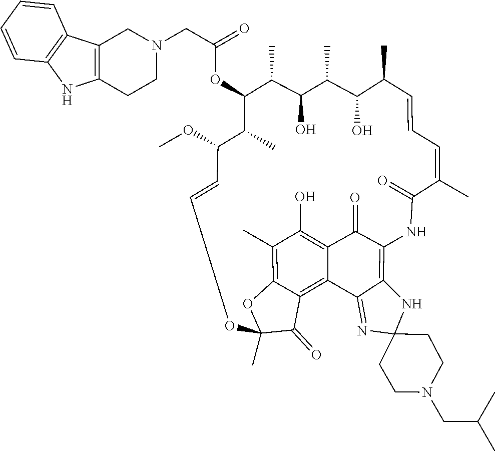 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCc3[nH]c5ccccc5c3C2)[C@@H]1C)C4=O] | 1016.52 | 17.92 | 1017.78 |
| 34 | 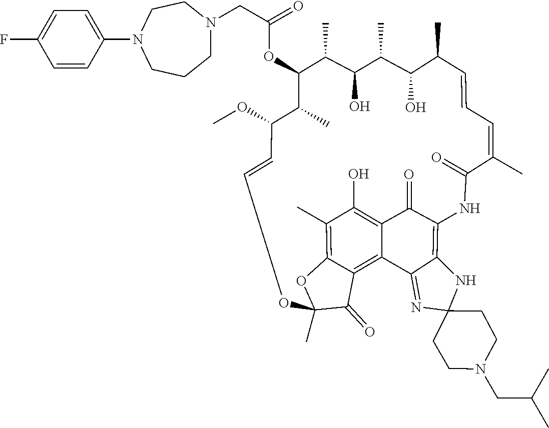 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCN(c3ccc(F)cc3)CC2)[C@@H]1C)C4=O] | 1038.54 | 19.47 | 1039.81 |
| 39 | 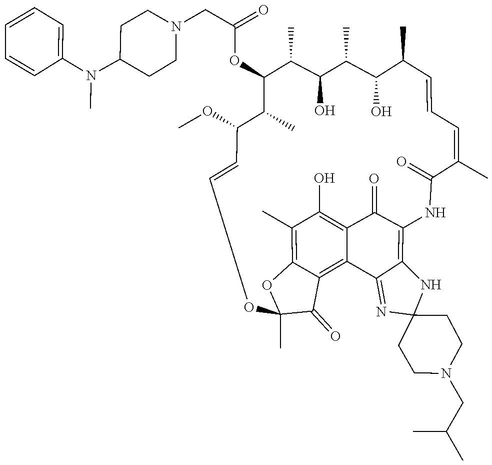 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)c3ccccc3)CC2)[C@@H]1C)C4=O] | 1034.57 | 19.85 | 1035.76 |
| 40 | 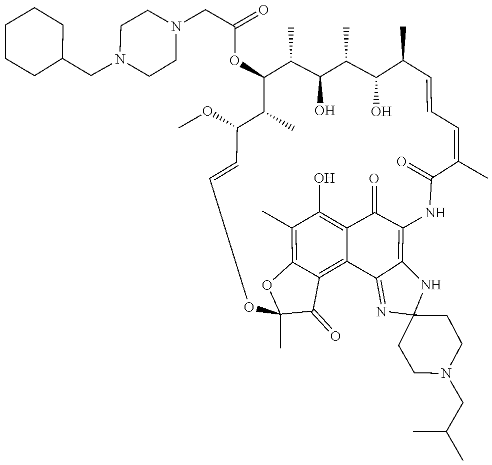 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(CC3CCCCC3)CC2)[C@@H]1C)C4=O] | 1026.60 | 20.25 | 1027.77 |

Additional analytical data (NMR):

### Cpd #39:

¹H NMR (300 MHz, CDCl₃): δ −0.07 (d, J=7.0 Hz, 3H), 0.58 (d, J=7.0 Hz, 3H), 0.82 (d, J=6.9 Hz, 3H), 0.93 (d, J=6.5 Hz, 6H), 1.00 (d, J=6.9 Hz, 3H), 1.37-1.47 (m, 1H), 1.65-2.11 (m, 11H), 1.73 (s, 3H), 2.03 (s, 3H), 2.14-2.38 (m, 5H), 2.32 (s, 3H), 2.59-2.80 (m, 2H), 2.76 (s, 3H), 2.90-3.25 (m, 7H), 3.06 (s, 3H), 3.30 (dd, J=7.5 Hz, 1.8 Hz, 1H), 3.39-3.71 (m, 4H), 4.81 (d, J=10.5 Hz, 1H), 5.12 (dd, J=12.5 Hz, 7.6 Hz, 1H), 5.98 (dd, J=15.5 Hz, 6.6 Hz, 1H), 6.16 (d, J=12.5 Hz, 1H), 6.20-6.41 (m, 2H), 6.69 (dd, J=7.4 Hz, 7.4 Hz, 1H), 6.77 (d, J=8.0 Hz, 2H), 7.16-7.25 (m, 2H), 8.22 (s, 1H), 9.09 (1H), 14.72 (s, 1H).

¹³C NMR (75 MHz, CDCl₃): δ 7.94, 9.02, 11.31, 11.57, 17.63, 20.51, 21.11, 21.12, 22.21, 26.03, 28.99, 29.07, 31.65, 33.29, 35.49, 36.39, 37.96, 38.49, 51.66, 51.70, 53.55, 53.79, 56.67, 57.10, 59.51, 66.49, 72.81, 73.58, 77.11, 81.28, 94.75, 104.87, 107.58, 109.12, 111.99, 113.85, 114.59, 115.69, 117.11, 124.26, 125.35, 129.39, 131.55, 133.22, 141.13, 142.23, 144.82, 150.29, 155.44, 168.45, 168.67, 171.68, 181.30, 192.70.

### Cpd #40:

¹H NMR (300 MHz, CDCl₃): δ −0.06 (d, J=6.9 Hz, 3H), 0.56 (d, J=6.4 Hz, 3H), 0.74-1.07 (m, 14H), 1.08-1.30 (m, 4H), 1.34-1.96 (m, 18H), 2.58-2.88 (m, 2H), 2.90-3.18 (m, 6H), 3.25-3.55 (m, 5H), 3.66 (d, J=9.5 Hz, 1H), 3.84 (s, 1H), 4.60 (d, J=10.2 Hz, 1H), 4.74 (d, J=3.03 Hz, 1H), 4.99 (dd, J=12.2, 6.0 Hz, 1H), 5.96-6.16 (m, 2H), 6.22 (d, J=10.5 Hz, 1H), 6.40 (dd, J=15.3, 10.7 Hz, 1H), 8.19 (s, 1H), 8.87 (s, 1H), 14.82 (s, 1H).

¹³C NMR (75 MHz, CDCl₃): δ 7.75, 8.90, 10.4, 11.6, 17.6, 20.4, 20.9, 21.6, 25.7, 26.1, 26.8, 31.8, 33.3, 35.0, 35.9, 37.6, 37.8, 38.5, 44.0, 51.5, 53.4, 53.6, 57.2, 65.6, 66.1, 73.3, 74.6, 79.5, 94.3, 104.9, 107.1, 108.6, 112.0, 114.1, 116.0, 124.4, 125.3, 130.6, 133.4, 141.3, 142.3, 143.1, 155.6, 156.2, 168.4, 171.7, 181.5, 192.7.

### Example 4

### General Procedure for the Preparation of Ammonium and Pyridinium Derivatives

A solution of Intermediate (I-2) in THF (0.07M) was prepared. The corresponding amine or pyridine (0.030 mmol, 1.5 eq) were dispensed in 9 mL Kimble glass tubes, and 300 μL of the solution of the rifabutin Intermediate in THF (corresponding to 20 mg, 0.020 mmol, 1 eq) was added. The reaction was stirred at 60° C. After 60 h, 600 μL of a solution of camphorsulphonic acid in water (0.5N) was added to the mixtures. The reaction was stirred at rt for 12 h. The mixture was then deposited on PoraPak Column (Rxn RP 6CC) previously equilibrated with water. The elution was carried out by adding 5 mL of water, 5 mL of a sat. aq. solution of NaHCO₃, 5 mL of a solution of water/ACN (90/10) and 10 mL of ACN. The 10 mL of ACN fractions were collected, frozen and freeze-dried to give the desired products as purple powders.

TABLE 5
Compounds (Cpd) of the Invention ammonium/pyridinium derivatives.

| Cpd | Structure | MW | Rt | [M + H]+ |
|---|---|---|---|---|
| 35 | 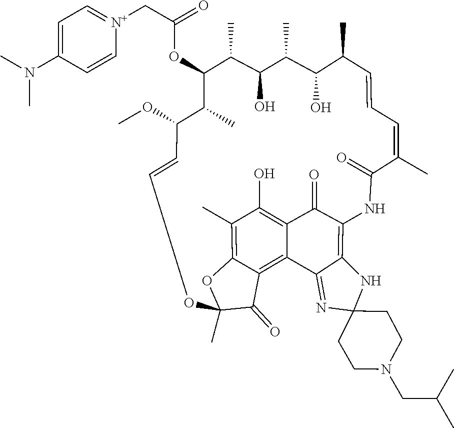 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccc(N(C)C)cc2)[C@@H]1C)C4=O] | 967.51 | 15.02 | 967.76 |
| 36 | 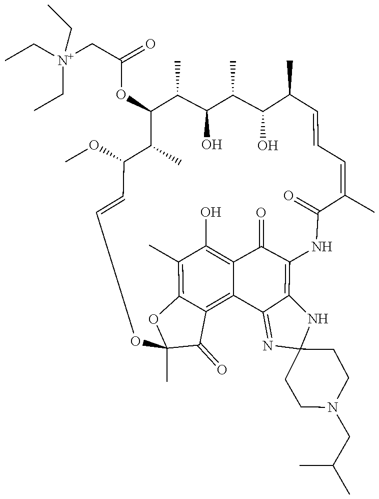 [SMILES: CC[N+](CC)(CC)CC(=O)O[C@H]1[C@H](C)[C@H](O)[C@H](C)[C@@H](O)[C@@H](C)/C=C/C=C(/C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O] | 946.55 | 14.70 | 946.68 |
| 37 | 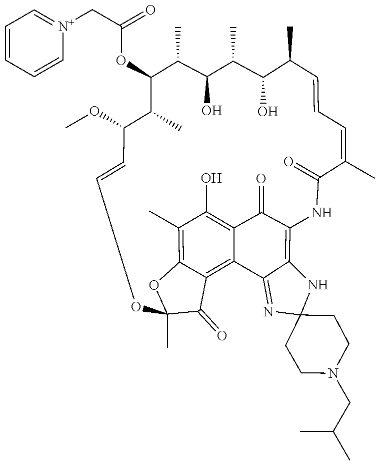 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccccc2)[C@@H]1C)C4=O] | 924.47 | 13.59 | 924.45 |
| 38 | 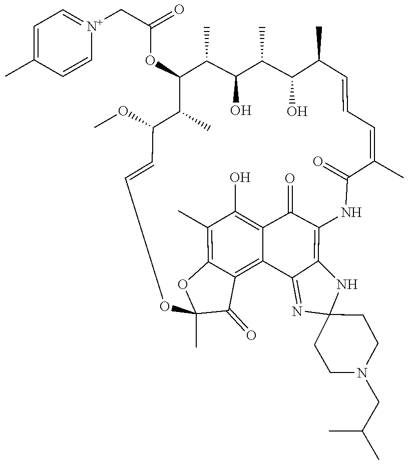 | 938.49 | 14.09 | 938.67 |

### Example 5

### Antibacterial Activity

MIC values were determined by broth microdilution method according to the CLSI guideline. Unless otherwise mentioned, MIC against A. baumannii was performed in RPMI medium supplemented with 10% FCS. MIC against M. abscessus, M avium, M. kansasii, M. xenopi, M. tuberculosis and M. smegmatis was performed in Middlebrook 7H9 broth supplemented with Middlebrook ADC growth supplement. All other MIC were performed in standard cation adjusted Mueller Hinton broth.

The following procedure applies to all species tested except M. avium, M. kansasii, M. xenopi, M. tuberculosis and M. smegmatis. From an overnight culture plate (for S. aureus and A. baumannii) or 3-4 days culture plate (for M. abscessus), cells were resuspended in 0.9% (w/v) saline solution and bacterial inoculum was prepared in the respective testing medium with 5×10⁵ CFU/mL. The appropriate volumes of a 10 mg/mL compound solution were directly dispensed in the 96-well assay plate using a digital dispenser to create two-fold dilution series from 32 to 0.002 μg/mL final concentration. 100 μL of bacterial suspension was finally added to the compounds. Plates were covered and incubated without shaking at 35° C. for 20 hours. Every experiment contained an antibiotic as quality control. MIC was determined visually as the lowest concentration of a compound that prevents visible growth of the bacteria and the plates were scanned for documentation.

A similar procedure was used for M. avium, M. kansasii, M. xenopi, M. tuberculosis and M. smegmatis, except that the inoculum was prepared from an exponentially growing culture and set to an OD₆₀₀ of 0.02 for M. tuberculosis and 0.002 for the other mycobacteria. Ten 3-fold serial dilutions of tested compounds were performed in duplicates into black Greiner 384-well clear bottom polystyrene plates (Greiner 781091) using an Echo 550 liquid handler (Labcyte). Plates were inoculated with 50 μl/well of the relevant bacterial cultures. The MIC was determined at 90% growth inhibition using a GFP reporter for M. tuberculosis and using the resazurin assay for the other mycobacteria. The incubation period depended on the mycobacterium tested: 7 days for M. avium, M. kansasii, and M. xenopi, 5 days for M. tuberculosis and 20 h for M. smegmatis. Plates were read using a Victor Multilabel plate reader (PerkinElmer).

Activity of Compounds of the Invention Against S. aureus, A. baumannii, M. abscessus, M. tuberculosis, M. smegmatis, M. kansasii and M. xenopi.

The in vitro activity of compounds described herein was determined against S. aureus (UAMS-1625 strain), A. baumannii (HUMC1 strain), M. abscessus (ATCC 19977), M. tuberculosis H37Rv (ATCC 27294), M. smegmatis (ATCC 700084), M. avium (ATCC 25291), M. kansasii (ATCC 12478) and M. xenopi (ATCC 19250). Results for S. aureus, A. baumannii, M. tuberculosis, M. abscessus, M. smegmatis, M. avium, M. kansasii and M. xenopi are given in Table 6, below.

TABLE 6
In vitro activity of described compounds against A. baumannii, S. aureus,
M. tuberculosis, M. abscessus, M. smegmatis, M. kansasii and M. xenopi.

| Compound | S. | A. | M. | M. | M. | M. | M. | M. |
|---|---|---|---|---|---|---|---|---|
| Number | aureus | baumannii | abscessus | smegmatis | xenopi | avium | kansasii | tuberculosis |
| Rifabutin | 0.016 | ≤0.002 | 8 | 0.14 | 0.01 | 0.15 | 0.001 | 0.0003 |
| 1 | 0.06 | ≤0.002 | 2 | | | | | |
| 2 | 0.06 | ≤0.002 | 2 | 0.03 | 0.08 | 1.2 | 0.02 | 0.02 |
| 3 | 0.03 | ≤0.002 | 1 | 0.03 | 0.07 | 1.2 | 0.01 | 0.008 |
| 4 | 0.06 | 0.004 | 4 | | | | | |
| 6 | 0.03 | ≤0.002 | 1 | 0.03 | 0.04 | 1.3 | 0.01 | 0.008 |
| 7 | 0.5 | 0.06 | 0.5 | | | | | |
| 8 | 0.06 | ≤0.002 | 1 | | | | | |
| 9 | 0.06 | ≤0.002 | 2 | | | | | |
| 10 | 0.03 | ≤0.002 | 1 | 0.1 | 0.01 | 1 | 0.015 | 0.008 |
| 11 | 0.06 | 0.008 | 2 | | | | | |
| 12 | 0.03 | ≤0.002 | 1 | 0.03 | 0.01 | 1.4 | 0.0055 | 0.003 |
| 13 | 0.03 | ≤0.002 | 2 | | | | | |
| 17 | 0.06 | 0.008 | 4 | | | | | |
| 18 | 0.5 | 0.03 | 0.5 | 0.09 | 0.01 | 1.3 | 0.01 | 0.002 |
| 19 | 4 | 0.125 | 0.5 | 0.24 | 0.03 | 4 | 0.015 | 0.003 |
| 20 | 0.5 | 0.03 | 0.25 | 0.1 | 0.03 | 0.5 | 0.01 | 0.003 |
| 21 | 4 | 0.125 | 2 | | | | | |
| 22 | 4 | 0.25 | 4 | | | | | |
| 23 | 0.125 | 0.004 | 1 | | | | | |
| 24 | 0.125 | 0.016 | 1 | 0.1 | 0.01 | 2 | 0.01 | 0.004 |
| 25 | 1 | 0.06 | 1 | 0.1 | 0.02 | 0.8 | 0.02 | 0.008 |
| 26 | 0.06 | ≤0.002 | 0.5 | 0.04 | 0.06 | 1.15 | 0.02 | 0.007 |
| 27 | 0.125 | 0.008 | 0.25 | | | | | |
| 28 | 0.125 | 0.016 | 0.125 | 0.1 | 0.03 | 3.9 | 0.01 | 0.003 |
| 29 | 0.5 | 0.03 | 0.25 | 0.09 | 0.01 | 0.6 | 0.01 | 0.003 |
| 30 | 0.06 | ≤0.002 | 1 | | | | | |
| 31 | 0.06 | 0.008 | 4 | | | | | |
| 32 | 0.03 | 0.008 | 2 | | | | | |
| 33 | 1 | 0.125 | 0.5 | 0.1 | 0.7 | 0.3 | 0.01 | 0.008 |
| 34 | 2 | 0.125 | 0.5 | 0.11 | 0.01 | 0.5 | 0.009 | 0.008 |
| 35 | 0.06 | ≤0.002 | 0.5 | 0.03 | 0.04 | 0.8 | 0.003 | 0.0025 |
| 36 | 0.125 | ≤0.002 | 2 | | | | | |
| 37 | 0.25 | 0.004 | 2 | | | | | |
| 38 | 0.25 | 0.004 | 2 | | | | | |
| 39 | 0.125 | ≤0.03 | 0.125 | 0.06 | 0.003 | 0.4 | 0.003 | 0.0001 |
| 40 | 0.25 | 0.125 | 0.125 | 0.02 | 0.05 | 0.19 | 0.008 | 0.004 |

1. A compound of Formula I or a pharmaceutically acceptable salt, tautomer, solvate, hydrate or enantiomer thereof: 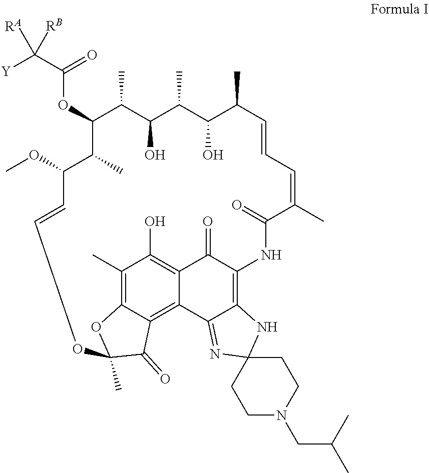
wherein:
R^{A} and R^{B} are each independently —H, —C₁-C₆ alkyl, or C₃-C₆ cycloalkyl;
Y is —NR¹R² or [NR⁵R⁵²R⁵³]⁺X⁻
R¹ and R² are each independently —H, —C₁-C₆ alkyl, —R³, or —C₁-C₆-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₆ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-15 membered heterocycloalkyl, wherein said heterocycloalkyl is optionally substituted with one or more —C₁-C₆ alkyl, halogen, —OR¹⁰, —NR¹¹R¹², oxo, —R¹³, or —C₁-C₆ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each —C₁-C₆ alkyl and —C₆-C₁₀ aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently a pharmaceutically acceptable anion, preferably X⁻ is F⁻, Cl⁻, Br⁻, I⁻, F₃CSO₃⁻, or H₃CC₆H₄SO₃⁻.

2. The compound according to claim 1, wherein R^{A} and R^{B} are each —H.

3. The compound of claim 1 or claim 2, wherein:
R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₆ alkyl;
R⁵ and R⁶ are independently, at each occurrence, —H or —C₁-C₆ alkyl;
or R¹ and R², together with the nitrogen atom to which they are attached, combine to form a 3-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen;
R¹⁴ is independently —H or —C₁-C₆ alkyl;
R⁵¹, R⁵² and R⁵³ are each independently selected from —H and —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently F⁻, Cl⁻, Br⁻, I⁻, F₃CSO₃⁻, or H₃CC₆H₄SO₃⁻.

4. The compound of Formula I, wherein Y is —NR¹R².

5. The compound of Formula I, wherein Y is —NR¹R² and:
R¹ and R² are each independently —H, —C₁-C₄ alkyl, —R³, or —C₁-C₄-alkylene-R³, wherein said alkyl and said alkylene are each independently optionally substituted with one or more halogen;
R³ is a 3-10 membered heterocycloalkyl, C₆-C₁₀ aryl, —OR⁴, or —NR⁵R⁶, wherein each heterocycloalkyl or aryl is optionally substituted with one or more halogen or —C₁-C₄ alkyl, wherein said alkyl is optionally substituted with one or more halogen;
R⁴ is independently —H or —C₁-C₆ alkyl; and
R⁵ and R⁶ are independently, at each occurrence, —H or —C₁-C₆ alkyl.

6. The compound of Formula I, wherein Y is —NR¹R² and
R¹ and R², together with the nitrogen atom to which they are attached, combine to form a 3-13 membered heterocycloalkyl, wherein said heterocycloalkyl is optionally substituted with one or more —C₁-C₄ alkyl, halogen, —OR¹⁰, —NR¹¹R¹², oxo, —R¹³, or —C₁-C₄ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen; and
R¹⁴ is independently —H or —C₁-C₆ alkyl.

7. The compound of Formula I, wherein Y is —NR¹R² and:
R¹ and R², together with the nitrogen atom to which they are attached, combine to form a piperidine, piperazine, or morpholine ring, wherein said piperidine, piperazine, or morpholine is optionally substituted with one or more —C₁-C₆ alkyl, halogen, —OR¹⁰, —NR¹¹R¹², oxo, R¹³, or —C₁-C₆ alkylene-R¹³; wherein each alkyl or alkylene is optionally substituted with one or more halogen or —OH;
R¹⁰ is independently —H, —C₁-C₆ alkyl, or —C₆-C₁₀ aryl, wherein each alkyl or aryl is optionally substituted with one or more halogen;
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each alkyl and aryl is optionally substituted with one or more halogen;
R¹³ is a C₃-C₆ cycloalkyl, 3-15 membered heterocycloalkyl, 5- to 10-membered heteroaryl, or C₆-C₁₀ aryl; wherein each cycloalkyl, heterocycloalkyl, heteroaryl, or aryl is optionally substituted with one or more oxo, halogen, —OR¹⁴, or —C₁-C₆ alkyl, wherein each alkyl is optionally substituted with one or more halogen; and
R¹⁴ is independently —H or —C₁-C₆ alkyl.

8. The compound of Formula I, wherein Y is —NR¹R² and:
R¹ and R², together with the nitrogen atom to which they are attached, combine to form an azetidine ring, wherein said azetidine ring is optionally substituted with one or more heteroaryl or —NR¹¹R¹²; and
R¹¹ and R¹² are each independently —H, —C₁-C₆ alkyl, —CO₂—C₁-C₆ alkyl, —C₆-C₁₀ aryl, or —SO₂—C₆-C₁₀ aryl; wherein each —C₁-C₆ alkyl and —C₆-C₁₀ aryl is optionally substituted with one or more halogen.

9. The compound of Formula I, wherein Y is —NR¹R² and
R¹ and R², together with the nitrogen atom to which they are attached, combine to form a heterocycle selected from 1,3,8-triaza-spiro[4.5]decane, decahydro-[1,6]naphthyridine, 2,3,4,5-tetrahydro-1H-pyrido[4,3-b]indole, or [1,4]diazepane; wherein said heterocycle is optionally substituted with one or more oxo, —C₁-C₆ alkylene R¹³, or R¹³; and
R¹³ is a C₆-C₁₀ aryl; wherein said aryl is optionally substituted with one or more halogen.

10. The compound of Formula I, wherein Y is [NR⁵¹R⁵²R⁵³]⁺X⁻, wherein R⁵¹, R⁵² and R⁵³ are each independently selected from —H and —C₁-C₆ alkyl;
or R⁵¹, R⁵² and R⁵³, together with the nitrogen atom to which they are attached, combine to form a 5 to 10-membered ring heteroaryl, wherein said heteroaryl is optionally substituted with one or more halogen, —C₁-C₆ alkyl, —OC₁-C₆ alkyl, or —N(C₁-C₆ alkyl)₂; and
X⁻ is independently F⁻, Cl⁻, Br⁻, I⁻, F₃CSO₃⁻, or H₃CC₆H₄SO₃⁻.

11. The compound according to claim 1, wherein Y is selected from the group consisting of

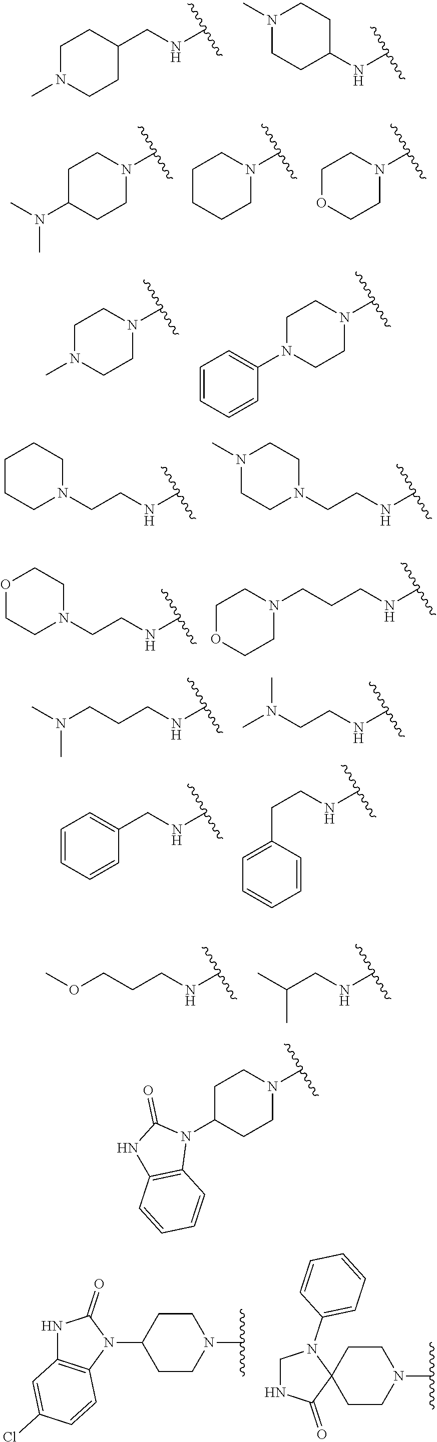

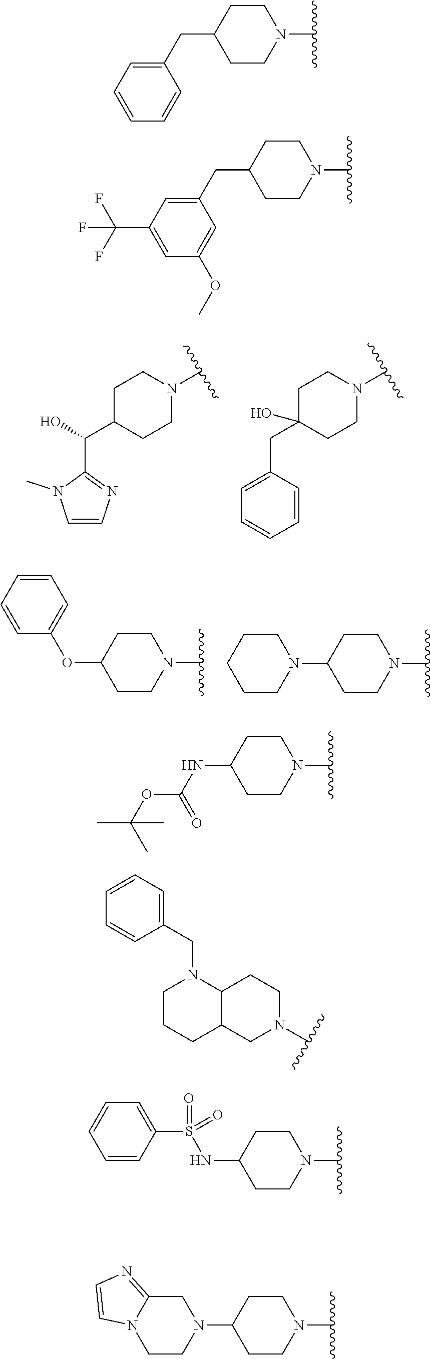

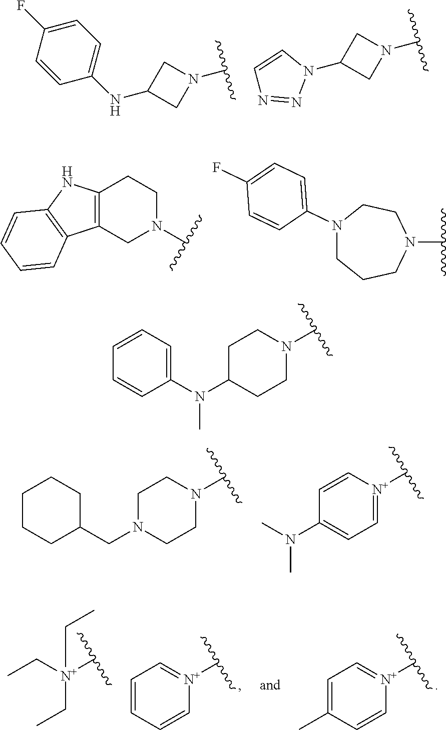

12. The compound according to claim 1, wherein the compound is selected from the group consisting of:

| Cpd | Structure |
|---|---|
| 1 | 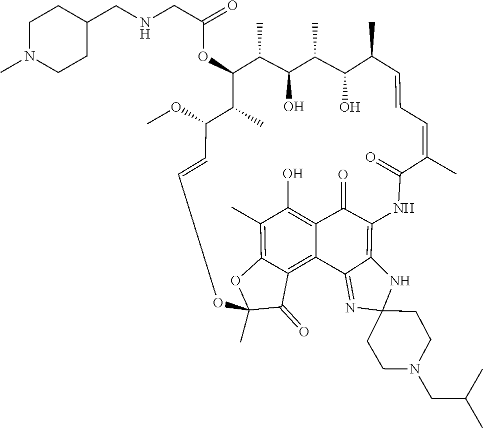 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCC2CCN(C)CC2)[C@@H]1C)C4=O] |
| 2 | 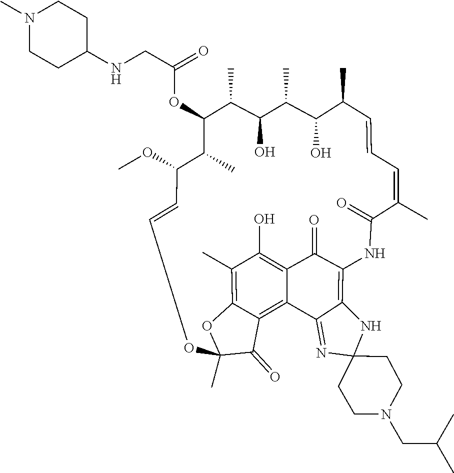 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNC2CCN(C)CC2)[C@@H]1C)C4=O] |
| 3 | 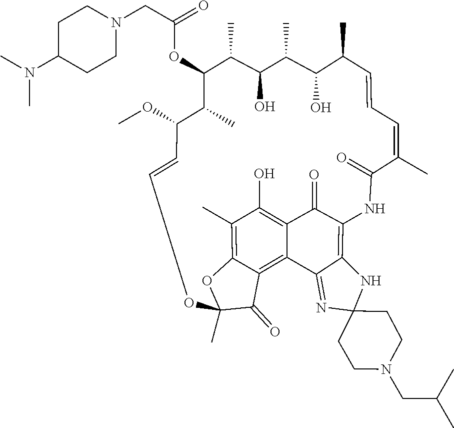 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)C)CC2)[C@@H]1C)C4=O] |
| 4 | 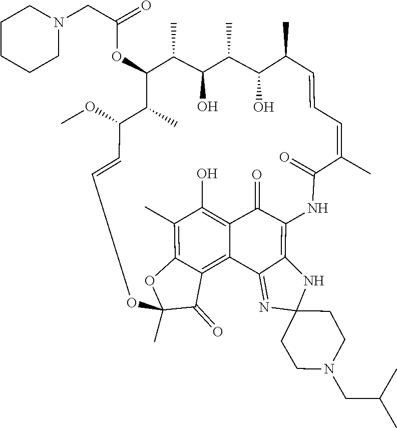 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCCC2)[C@@H]1C)C4=O] |
| 5 | 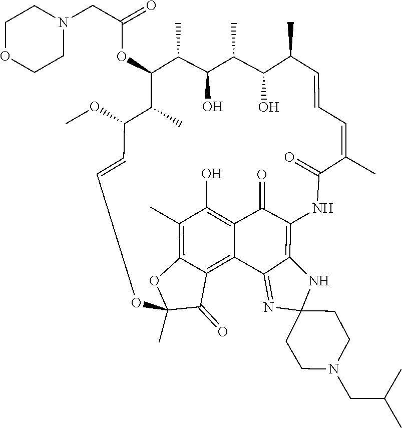 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCOCC2)[C@@H]1C)C4=O] |
| 6 | 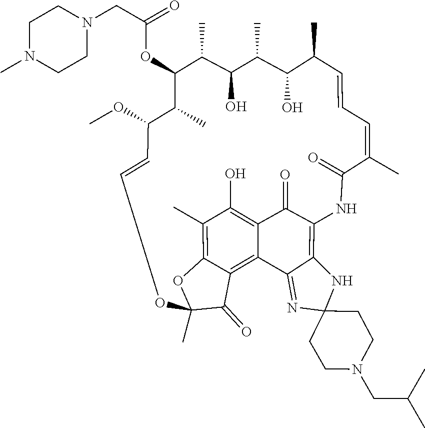 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(C)CC2)[C@@H]1C)C4=O] |
| 7 | 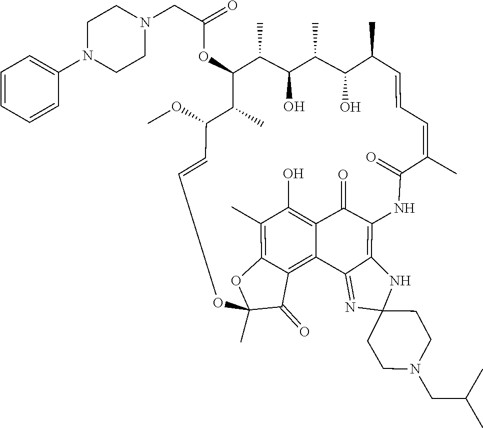 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(c3ccccc3)CC2)[C@@H]1C)C4=O] |
| 8 | 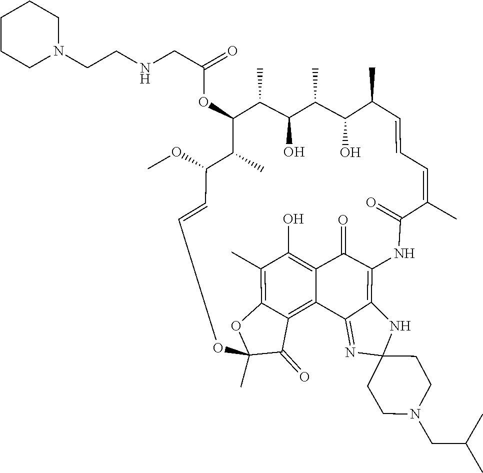 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCCCC2)[C@@H]1C)C4=O] |
| 9 | 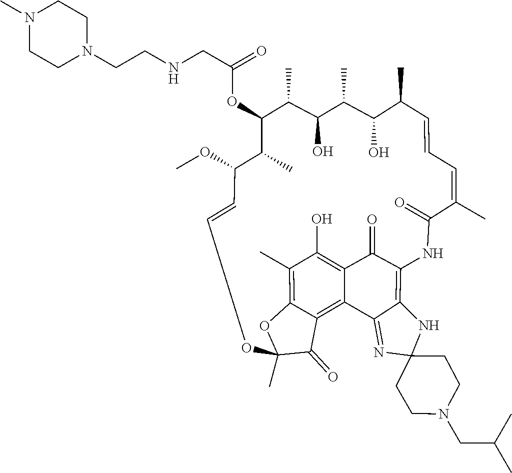 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCN(C)CC2)[C@@H]1C)C4=O] |
| 10 | 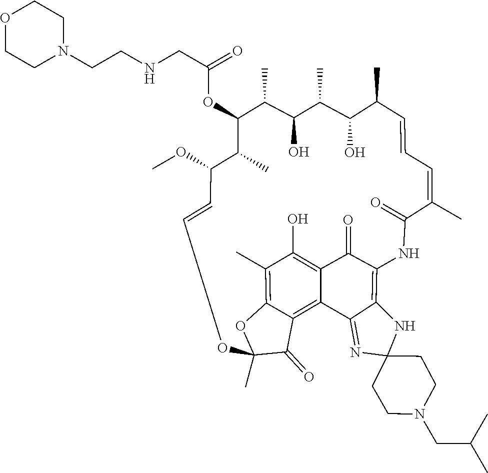 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCOCC2)[C@@H]1C)C4=O] |
| 11 | 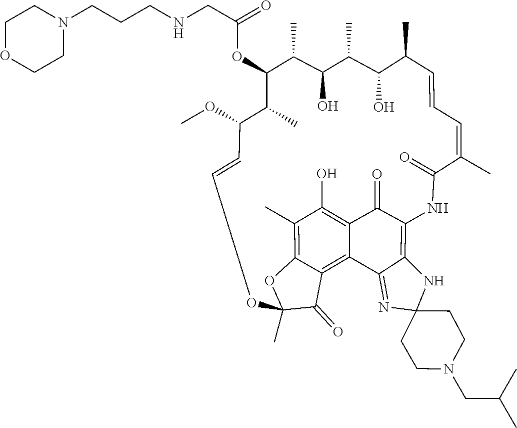 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN2CCOCC2)[C@@H]1C)C4=O] |
| 12 | 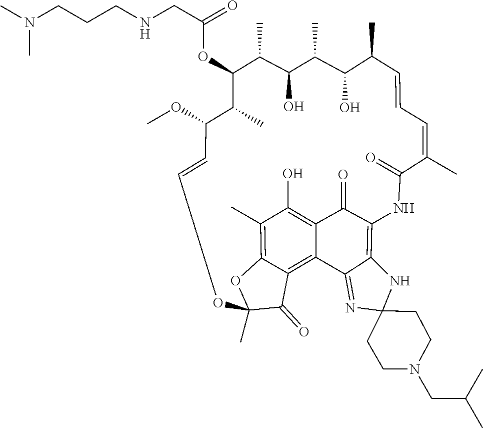 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN(C)C)[C@@H]1C)C4=O] |
| 13 | 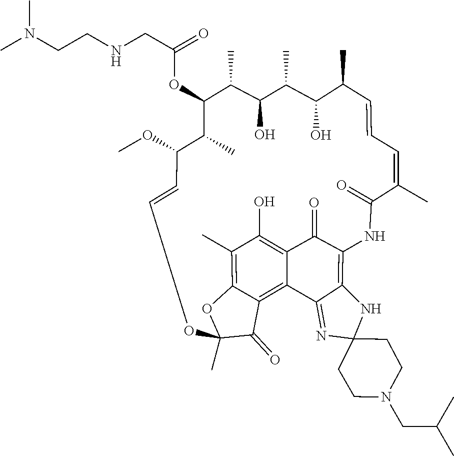 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN(C)C)[C@@H]1C)C4=O] |
| 14 | 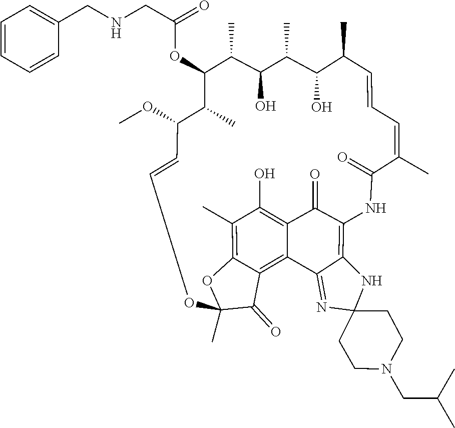 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCc2ccccc2)[C@@H]1C)C4=O] |
| 15 | 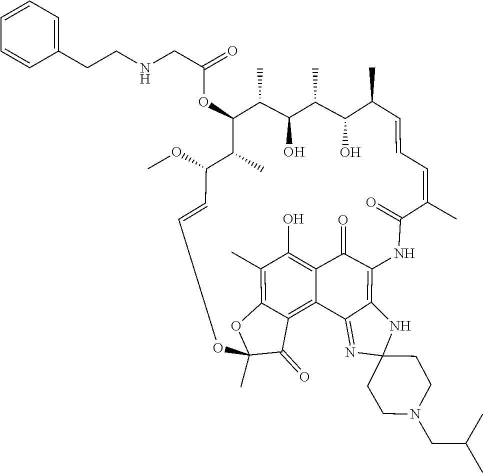 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCc2ccccc2)[C@@H]1C)C4=O] |
| 16 | 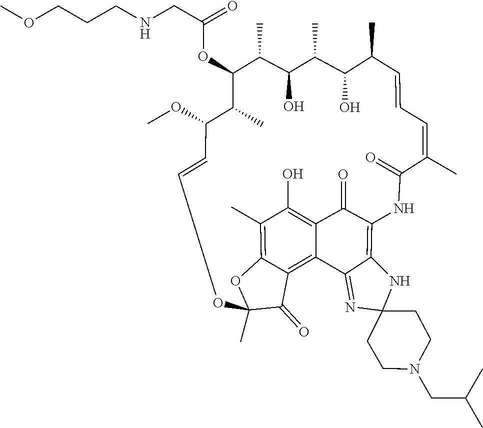 [SMILES: COCCCNCC(=O)O[C@H]1[C@H](C)[C@H](O)[C@H](C)[C@@H](O)[C@@H](C)/C=C/C=C(/C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O] |
| 17 | 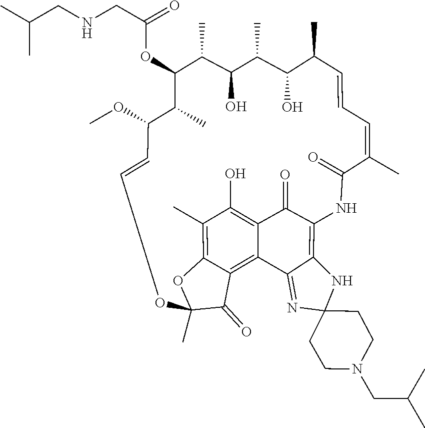 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCC(C)C)[C@@H]1C)C4=O] |
| 18 | 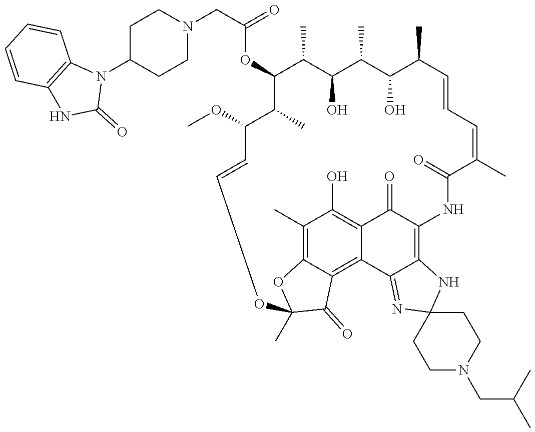 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5ccccc53)CC2)[C@@H]1C)C4=O] |
| 19 | 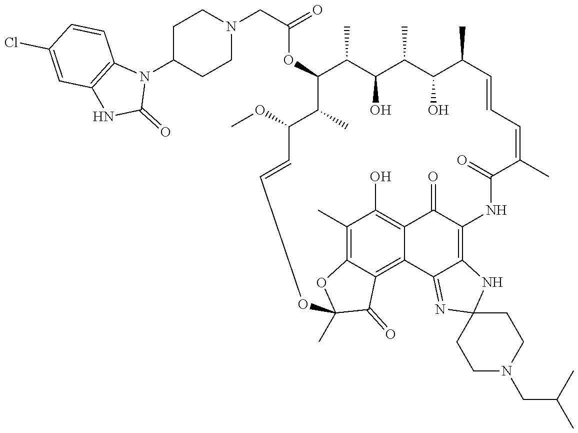 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5cc(Cl)ccc53)CC2)[C@@H]1C)C4=O] |
| 20 | 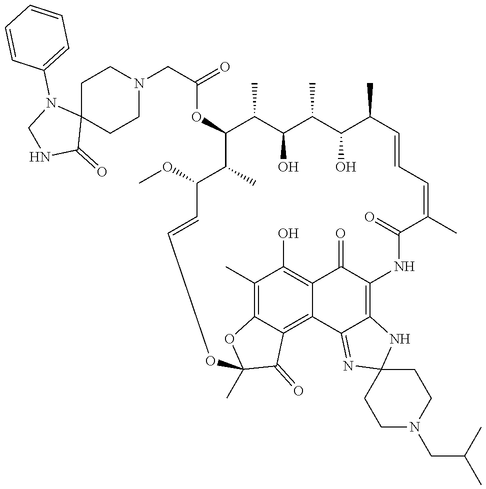 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3(CC2)C(=O)NCN3c2ccccc2)[C@@H]1C)C4=O] |
| 21 | 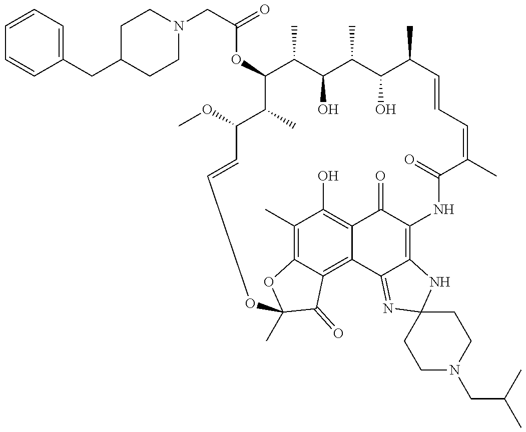 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Cc3ccccc3)CC2)[C@@H]1C)C4=O] |
| 22 | 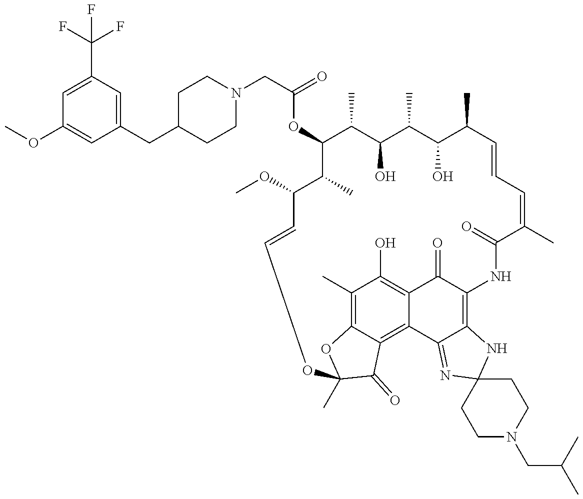 [SMILES: COc1cc(CC2CCN(CC(=O)O[C@H]3[C@H](C)[C@H](O)[C@H](C)[C@@H](O)[C@@H](C)/C=C/C=C(/C)C(=O)NC4=C5NC6(CCN(CC(C)C)CC6)N=C5c5c(c(O)c(C)c6c5C(=O)[C@@](C)(O/C=C/[C@H](OC)[C@H]3C)O6)C4=O)CC2)cc(C(F)(F)F)c1] |
| 23 | 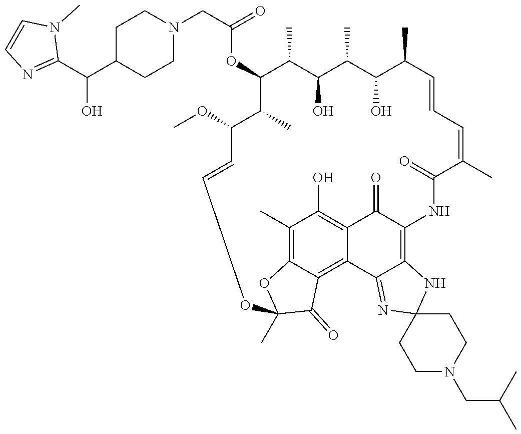 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(C(O)c3nccn3C)CC2)[C@@H]1C)C4=O] |
| 24 | 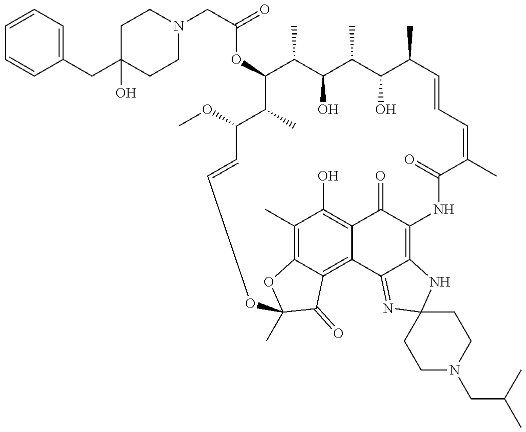 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(O)(Cc3ccccc3)CC2)[C@@H]1C)C4=O] |
| 25 | 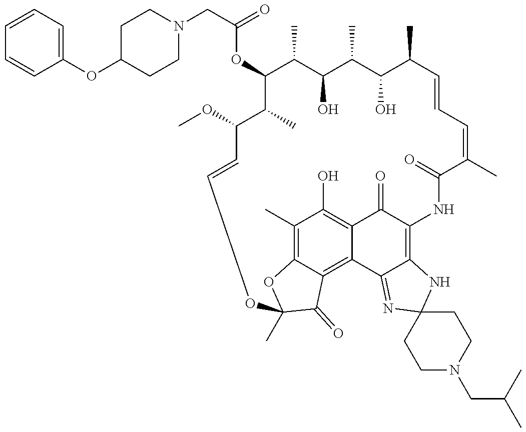 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Oc3ccccc3)CC2)[C@@H]1C)C4=O] |
| 26 | 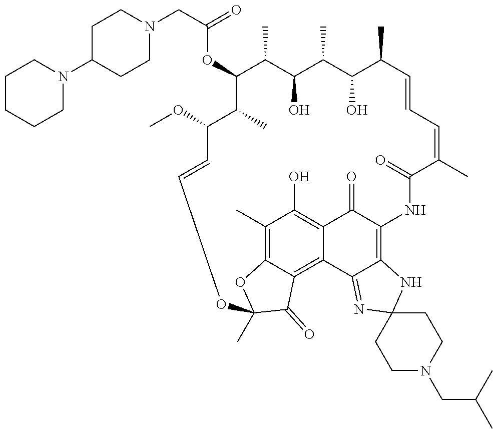 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCCCC3)CC2)[C@@H]1C)C4=O] |
| 27 | 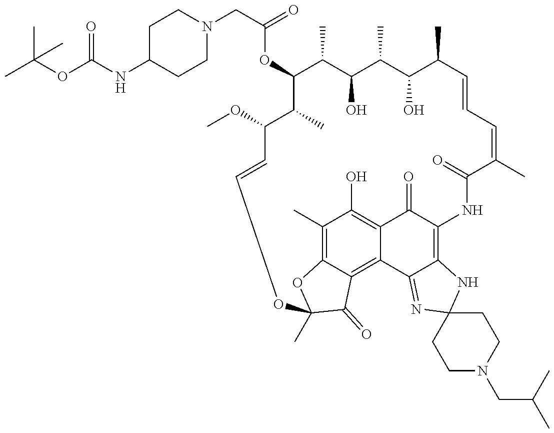 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NC(=O)OC(C)(C)C)CC2)[C@@H]1C)C4=O] |
| 28 | 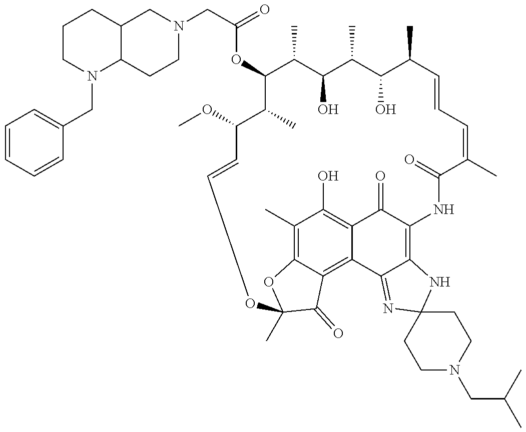 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3C(CCCN3Cc3ccccc3)C2)[C@@H]1C)C4=O] |
| 29 | 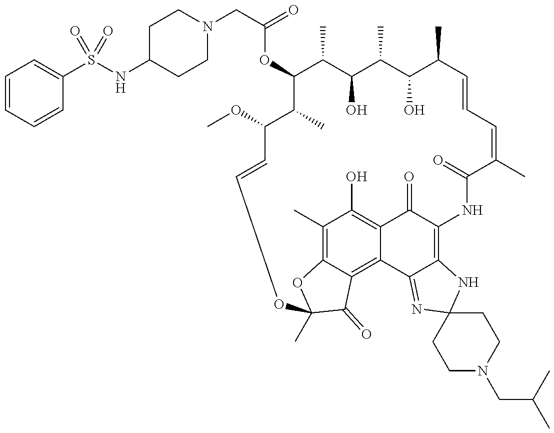 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NS(=O)(=O)c3ccccc3)CC2)[C@@H]1C)C4=O] |
| 30 | 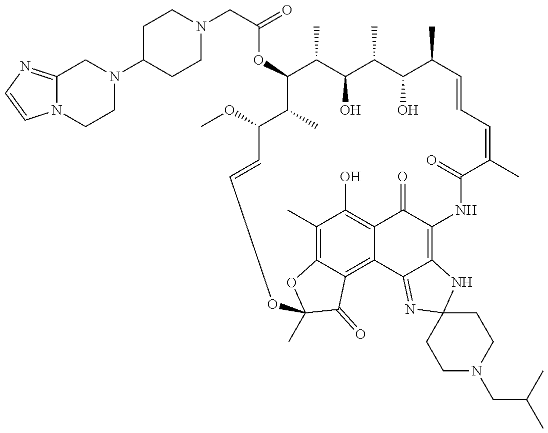 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCn5ccnc5C3)CC2)[C@@H]1C)C4=O] |
| 31 | 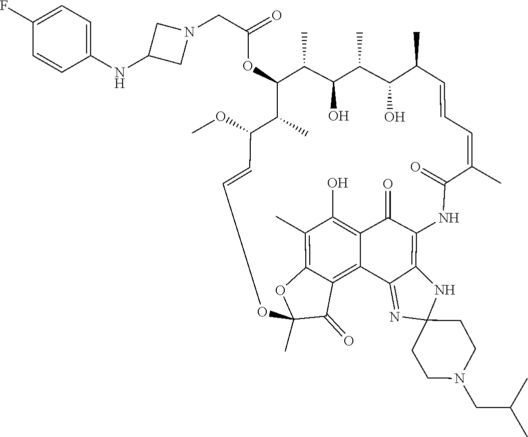 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CC(Nc3ccc(F)cc3)C2)[C@@H]1C)C4=O] |
| 32 | 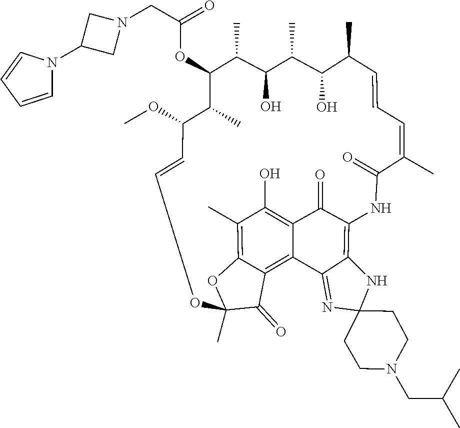 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CC(n3cccc3)C2)[C@@H]1C)C4=O] |
| 33 | 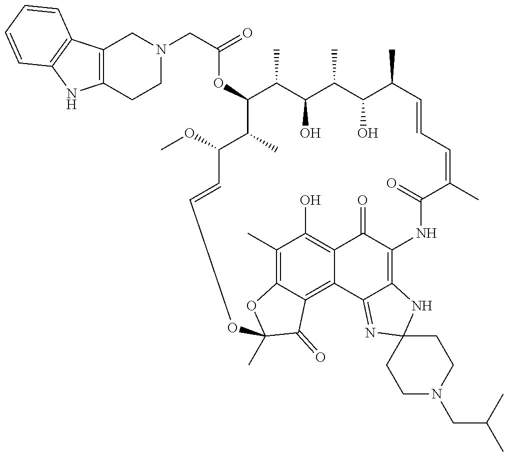 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCc3[nH]c5ccccc5c3C2)[C@@H]1C)C4=O] |
| 34 | 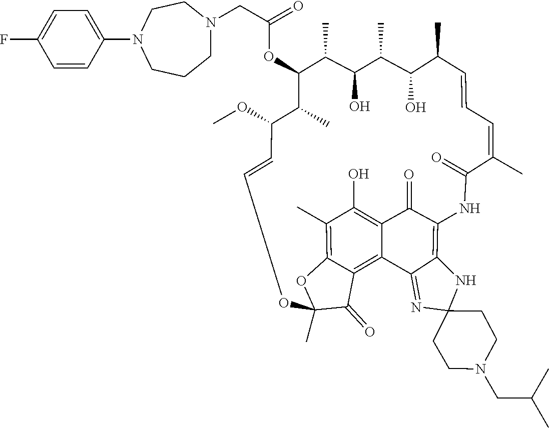 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCN(c3ccc(F)cc3)CC2)[C@@H]1C)C4=O] |
| 35 | 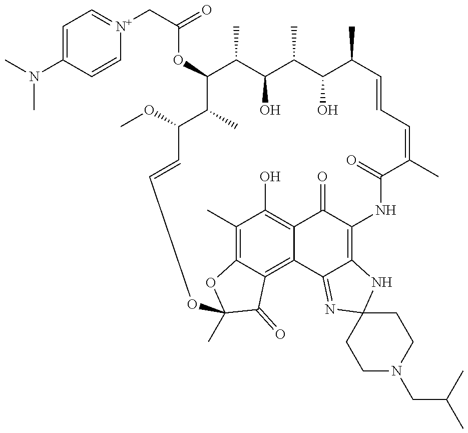 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccc(N(C)C)cc2)[C@@H]1C)C4=O] |
| 36 | 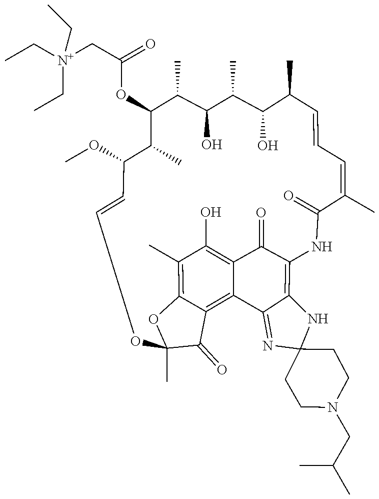 [SMILES: CC[N+](CC)(CC)CC(=O)O[C@H]1[C@H](C)[C@H](O)[C@H](C)[C@@H](O)[C@@H](C)/C=C/C=C(/C)C(=O)NC2=C3NC4(CCN(CC(C)C)CC4)N=C3c3c(c(O)c(C)c4c3C(=O)[C@@](C)(O/C=C/[C@H](OC)[C@H]1C)O4)C2=O] |
| 37 | 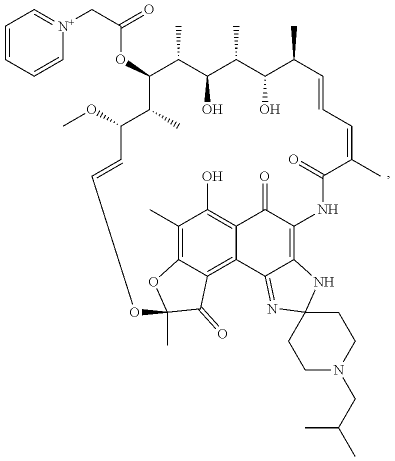 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccccc2)[C@@H]1C)C4=O] |
| 38 | 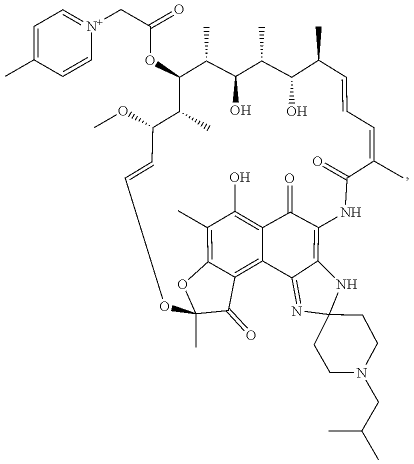 |
| 39 | 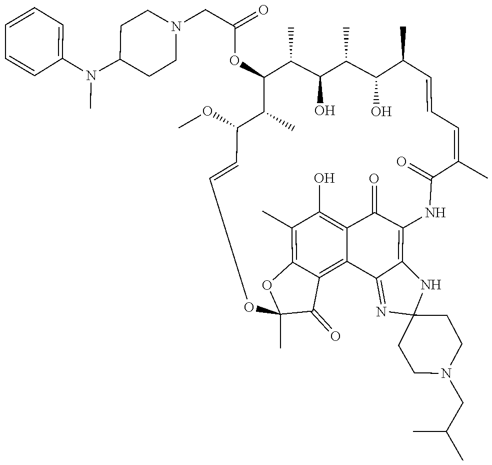 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)c3ccccc3)CC2)[C@@H]1C)C4=O] |
| | and |
| 40 | 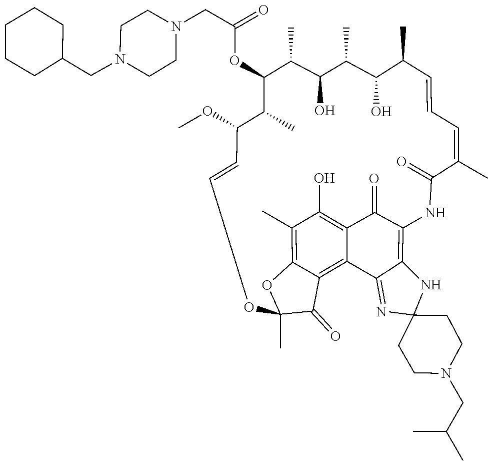 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(CC3CCCCC3)CC2)[C@@H]1C)C4=O] |

13. The compound according to claim 1, wherein the compound is selected from the group consisting of:

| Cpd | Structure |
|---|---|
| 3 | 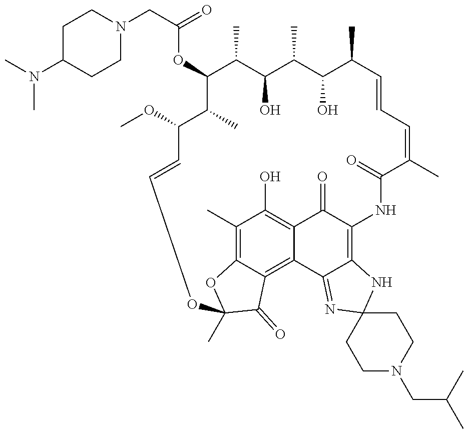 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)C)CC2)[C@@H]1C)C4=O] |
| 6 | 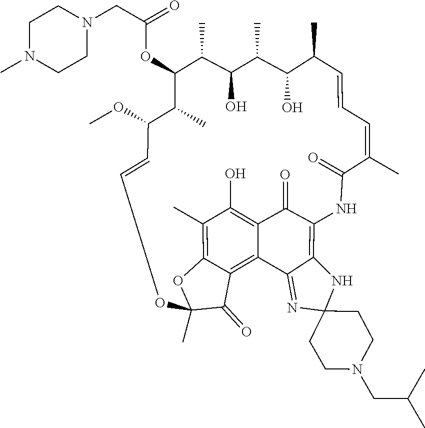 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(C)CC2)[C@@H]1C)C4=O] |
| 7 | 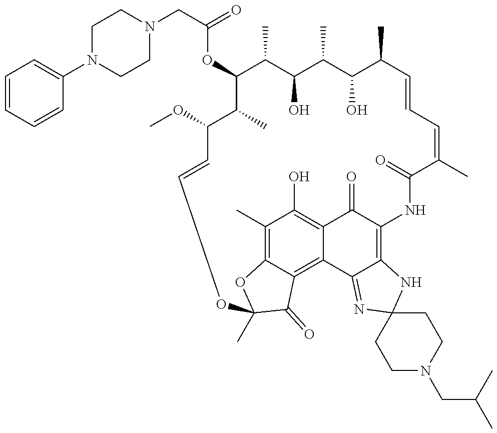 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(c3ccccc3)CC2)[C@@H]1C)C4=O] |
| 8 | 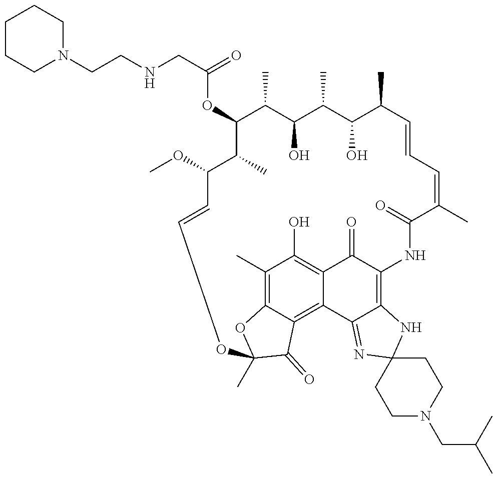 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCCCC2)[C@@H]1C)C4=O] |
| 10 | 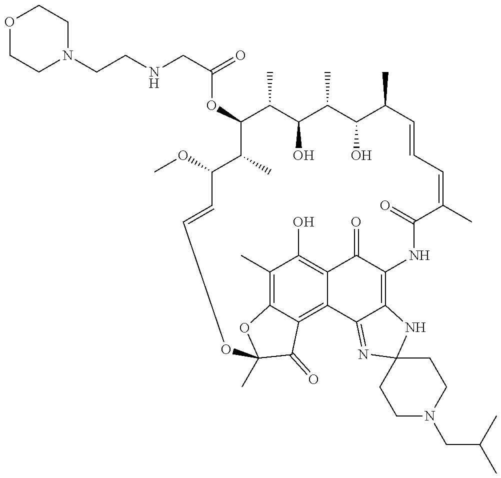 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCN2CCOCC2)[C@@H]1C)C4=O] |
| 12 | 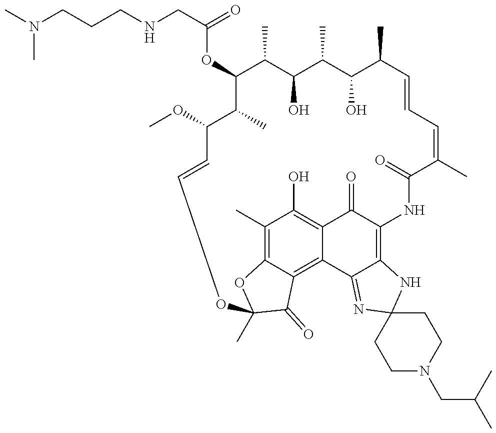 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CNCCCN(C)C)[C@@H]1C)C4=O] |
| 18 | 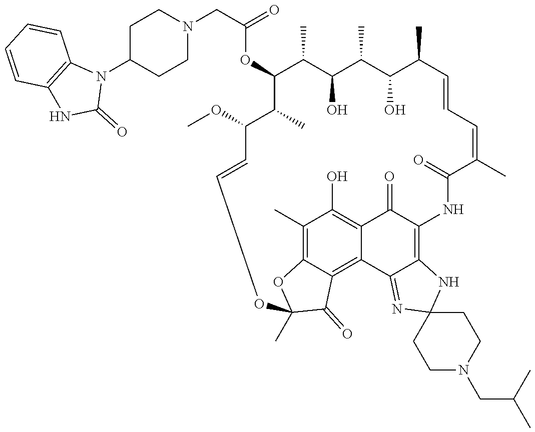 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5ccccc53)CC2)[C@@H]1C)C4=O] |
| 19 | 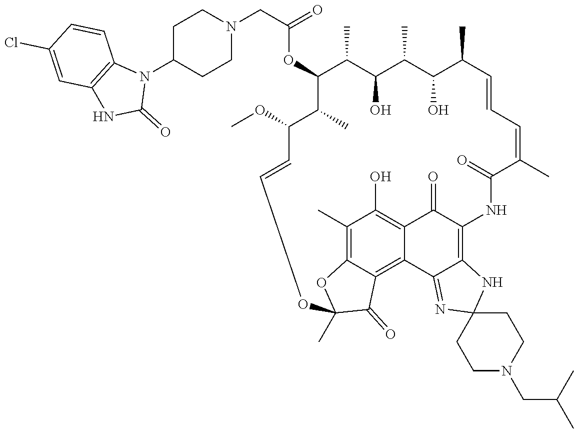 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(n3c(=O)[nH]c5cc(Cl)ccc53)CC2)[C@@H]1C)C4=O] |
| 20 | 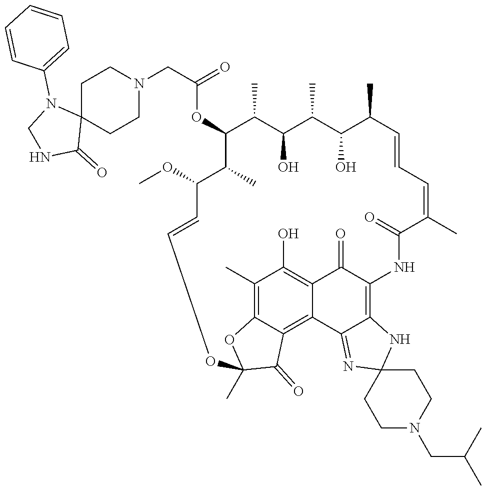 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3(CC2)C(=O)NCN3c2ccccc2)[C@@H]1C)C4=O] |
| 23 | 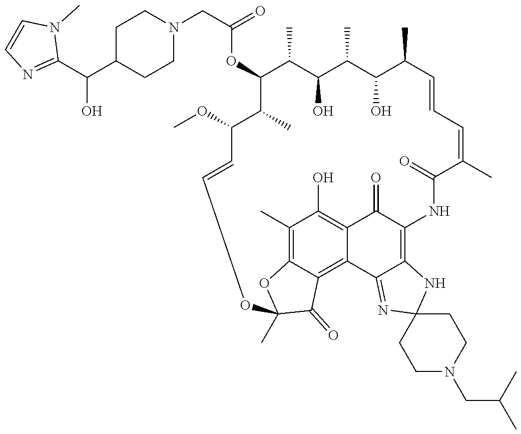 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(C(O)c3nccn3C)CC2)[C@@H]1C)C4=O] |
| 24 | 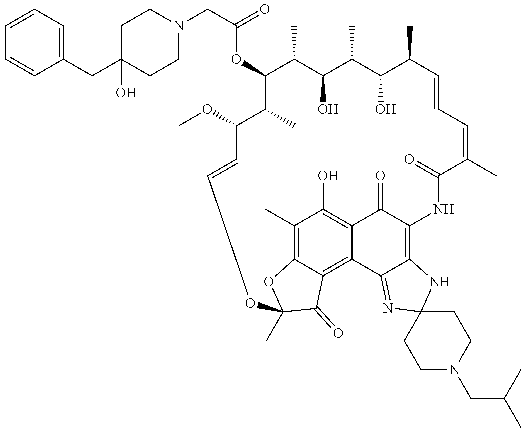 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(O)(Cc3ccccc3)CC2)[C@@H]1C)C4=O] |
| 25 | 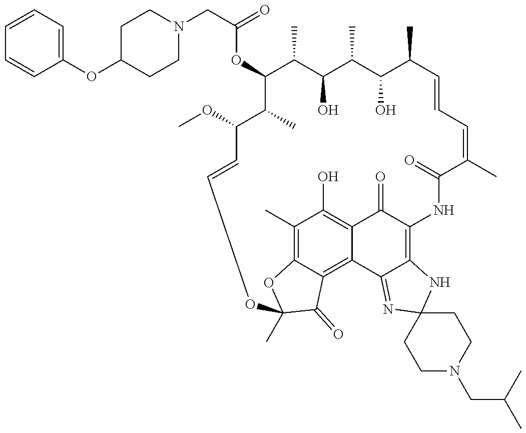 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(Oc3ccccc3)CC2)[C@@H]1C)C4=O] |
| 26 | 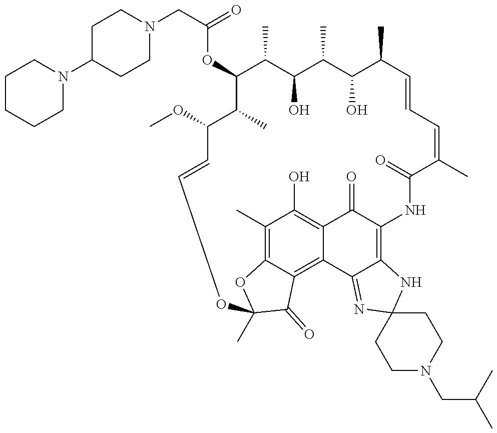 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCCCC3)CC2)[C@@H]1C)C4=O] |
| 27 | 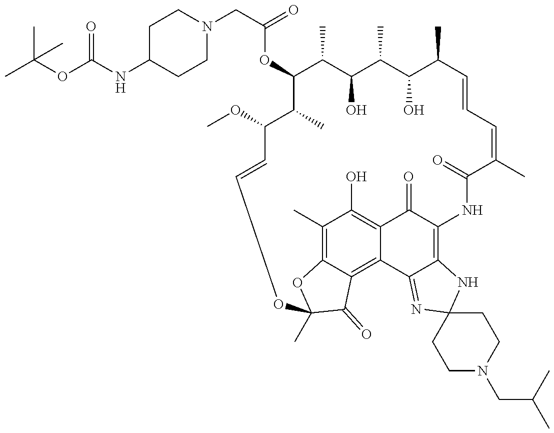 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NC(=O)OC(C)(C)C)CC2)[C@@H]1C)C4=O] |
| 28 | 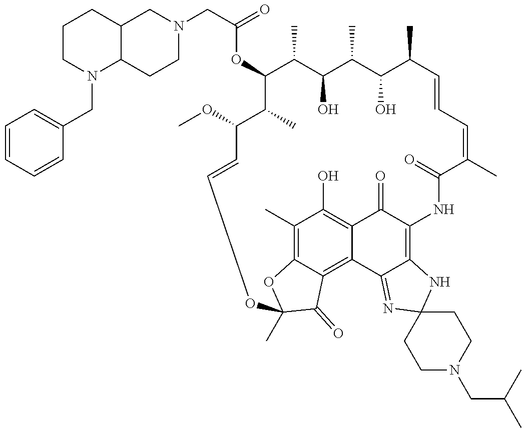 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC3C(CCCN3Cc3ccccc3)C2)[C@@H]1C)C4=O] |
| 29 | 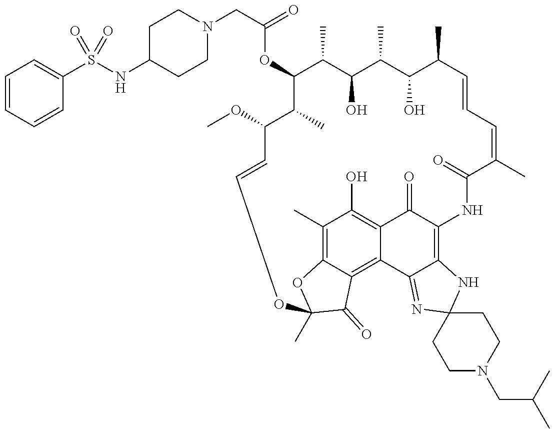 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(NS(=O)(=O)c3ccccc3)CC2)[C@@H]1C)C4=O] |
| 30 | 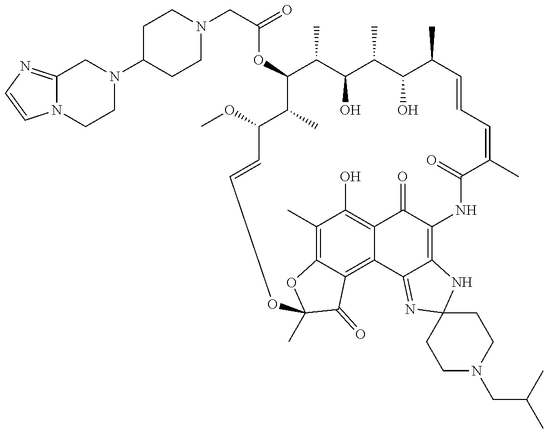 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N3CCn5ccnc5C3)CC2)[C@@H]1C)C4=O] |
| 33 | 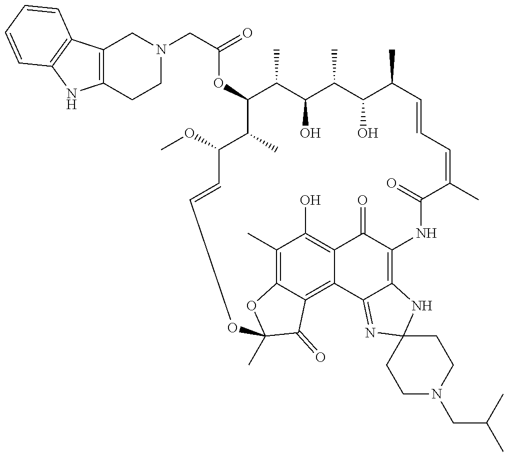 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCc3[nH]c5ccccc5c3C2)[C@@H]1C)C4=O] |
| 34 | 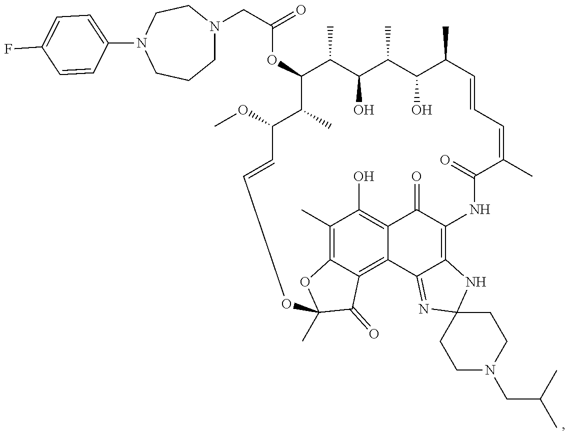 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCCN(c3ccc(F)cc3)CC2)[C@@H]1C)C4=O] |
| 35 | 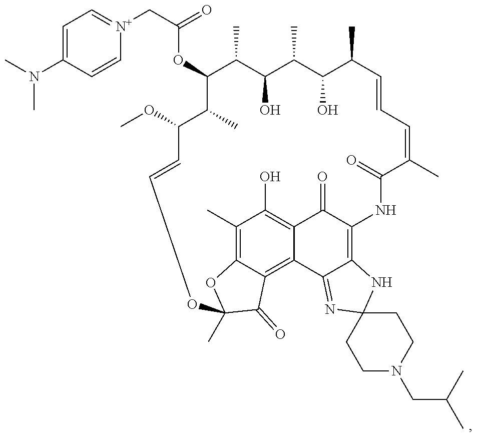 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)C[n+]2ccc(N(C)C)cc2)[C@@H]1C)C4=O] |
| 39 | 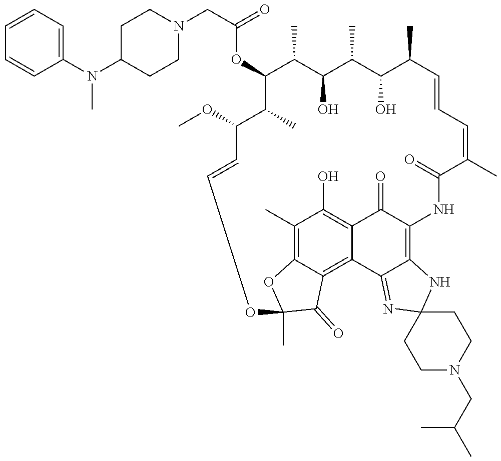 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCC(N(C)c3ccccc3)CC2)[C@@H]1C)C4=O] |
| | and |
| 40 | 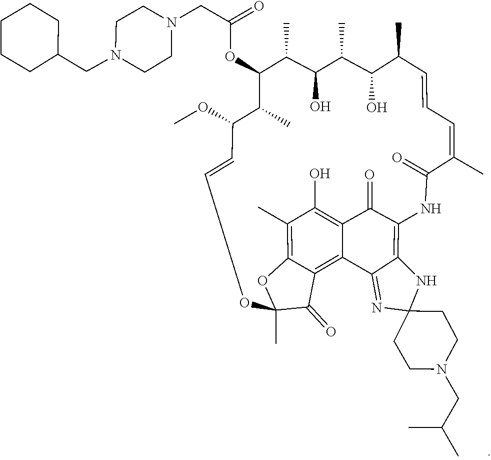 [SMILES: CO[C@H]1/C=C/O[C@@]2(C)Oc3c(C)c(O)c4c(c3C2=O)C2=NC3(CCN(CC(C)C)CC3)NC2=C(NC(=O)/C(C)=C\C=C\[C@H](C)[C@H](O)[C@@H](C)[C@@H](O)[C@@H](C)[C@H](OC(=O)CN2CCN(CC3CCCCC3)CC2)[C@@H]1C)C4=O] |

14. A pharmaceutical composition comprising at least one compound according to any of the preceding claims, or a pharmaceutically acceptable salt, tautomer, solvate or hydrate thereof, and a pharmaceutically acceptable excipient.

15. A compound according to any of the claims 1 to 13 or a pharmaceutically acceptable salt, tautomer, solvate or hydrate thereof, or a pharmaceutical composition according to claim 14, for use as a medicament.
